SEQUENCE LISTING (35 sequences)

SEQ ID NO: 1 - protein, 8 aa, Artificial Sequence
  Description of Artificial Sequence Synthetic peptide
GFTFNKYA

SEQ ID NO: 2 - protein, 10 aa, Artificial Sequence
  Description of Artificial Sequence Synthetic peptide
IRSKYNNYAT

SEQ ID NO: 3 - protein, 16 aa, Artificial Sequence
  Description of Artificial Sequence Synthetic peptide
VRHGNFGNSYISYWAY

SEQ ID NO: 4 - protein, 9 aa, Artificial Sequence
  Description of Artificial Sequence Synthetic peptide
TGAVTSGNY

SEQ ID NO: 5 - protein, 3 aa, Artificial Sequence
  Description of Artificial Sequence Synthetic peptide
GTK

SEQ ID NO: 6 - protein, 9 aa, Artificial Sequence
  Description of Artificial Sequence Synthetic peptide
VLWYSNRWV

SEQ ID NO: 7 - protein, 109 aa, Artificial Sequence
  Description of Artificial Sequence Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 8 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 9 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 8 aa, Artificial Sequence
  Description of Artificial Sequence Synthetic peptide
GYTFTNYG

SEQ ID NO: 11 - protein, 8 aa, Artificial Sequence
  Description of Artificial Sequence Synthetic peptide
INTYTGEP

SEQ ID NO: 12 - protein, 14 aa, Artificial Sequence
  Description of Artificial Sequence Synthetic peptide
ARGGFGSSYWYFDV

SEQ ID NO: 13 - protein, 6 aa, Artificial Sequence
  Description of Artificial Sequence Synthetic peptide
QDVSIA

SEQ ID NO: 14 - protein, 3 aa, Artificial Sequence
  Description of Artificial Sequence Synthetic peptide
SAS

SEQ ID NO: 15 - protein, 9 aa, Artificial Sequence
  Description of Artificial Sequence Synthetic peptide
QQHYITPLT

SEQ ID NO: 16 - protein, 214 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 224 aa, Artificial Sequence
  Description of Artificial Sequence Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 5 aa, Artificial Sequence
  Description of Artificial Sequence Synthetic peptide
GGGGS

SEQ ID NO: 19 - protein, 15 aa, Artificial Sequence
  Description of Artificial Sequence Synthetic peptide
GGGGSGGGGSGGGGS

SEQ ID NO: 20 - protein, 214 aa, Artificial Sequence; identical to SEQ ID NO: 16
  Description of Artificial Sequence Synthetic polypeptide

SEQ ID NO: 21 - protein, 478 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 468 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 224 aa, Artificial Sequence; identical to SEQ ID NO: 17
  Description of Artificial Sequence Synthetic polypeptide

SEQ ID NO: 24 - protein, 9 aa, Artificial Sequence
  Description of Artificial Sequence Synthetic peptide
TGAVTTSNY

SEQ ID NO: 25 - protein, 3 aa, Artificial Sequence
  Description of Artificial Sequence Synthetic peptide
GTN

SEQ ID NO: 26 - protein, 9 aa, Artificial Sequence
  Description of Artificial Sequence Synthetic peptide
ALWYSNLWV

SEQ ID NO: 27 - protein, 8 aa, Artificial Sequence
  Description of Artificial Sequence Synthetic peptide
GFTFNTYA

SEQ ID NO: 28 - protein, 10 aa, Artificial Sequence
  Description of Artificial Sequence Synthetic peptide
IRSKYNNYAT

SEQ ID NO: 29 - protein, 16 aa, Artificial Sequence
  Description of Artificial Sequence Synthetic peptide
VRHGNFGNSYVSWFAY

SEQ ID NO: 30 - protein, 109 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 31 - protein, 126 aa, Artificial Sequence
  Description of Artificial Sequence Synthetic polypeptide
EVQLVESGGGLVQPGGSLKLSCAASGFTFNTYAMNWVRQAPGKGLEWVARIRSKYNNYATYYADSVKSDRFTISRDDSKNTAYLQMNNLKTEDTAVYYCVRHGNFGNSYVSWFAYWGQGTLVTVSS

SEQ ID NO: 32 - protein, 249 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 33 - protein, 478 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 468 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 224 aa, Artificial Sequence; identical to SEQ ID NO: 17
  Description of Artificial Sequence Synthetic polypeptide

(19) United States
(12) Patent Application Publication
(10) Pub. No.: US 2024/0092931 A1
(43) Pub. Date: Mar. 21, 2024

(54) ANTIBODIES TARGETING TROP2 AND CD3 AND USES THEREOF

(71) Applicant: JANUX THERAPEUTICS, INC., San Diego, CA (US)

(72) Inventors: David CAMPBELL, San Diego, CA (US); Thomas R. DIRAIMONDO, San Diego, CA (US)

(21) Appl. No.: 18/041,876

(22) PCT Filed: Aug. 23, 2021
(86) PCT No.: PCT/US21/47191

§ 371 Date: Feb. 16, 2023

Related U.S. Application Data
(60) Provisional application No. 63/069,518, filed on Aug. 24, 2020.
(60) Provisional application No. 63/189,461, filed on May 17, 2021.

(51) Int. Cl. C07K 16/30; C07K 16/28

(52) U.S. Cl. CPC C07K 16/30; C07K 16/2809; A61K 2039/505

(57) ABSTRACT

Provided herein are antibodies that selectively bind to TROP2 and CD3, pharmaceutical compositions thereof, as well as nucleic acids, and methods for making and discovering the same.

### CROSS-REFERENCE

This application claims the benefit of U.S. Provisional Application No. 63/069,518, filed Aug. 24, 2020, and U.S. Provisional Application No. 63/189,461, filed May 17, 2021, each of which is incorporated herein by reference.

### SEQUENCE LISTING

The instant application contains a Sequence Listing which has been submitted electronically in ASCII format and is hereby incorporated by reference in its entirety. Said ASCII copy, created on Aug. 20, 2021, is named 52426_722_601_SL.txt and is 40,768 bytes in size.

### SUMMARY

Disclosed herein, in certain embodiments, are isolated polypeptide complexes according to the following formula:

A-L-B (Formula I)

wherein A comprises a single chain variable fragment (scFv) that binds to CD3; B comprises an antigen binding fragment (Fab) or Fab′ that binds to TROP2 wherein the Fab or Fab′ comprises a Fab light chain polypeptide comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B. In some instances, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3. In some instances, the scFv comprises a scFv light chain variable domain and a scFv heavy chain variable domain. In some instances, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3. In some instances, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3. In some instances, the Fab heavy chain polypeptide comprises an amino acid sequence that has at least 80% sequence identity to the amino acid sequence according to SEQ ID NO: 17. In some instances, the Fab heavy chain polypeptide comprises an amino acid sequence of at least 100 consecutive amino acid residues of SEQ ID NO: 17. In some instances, the Fab heavy chain polypeptide comprises an amino acid sequence of at least 200 consecutive amino acid residues of SEQ ID NO: 17. In some instances, the Fab heavy chain polypeptide comprises an amino acid sequence of at least 200 consecutive amino acid residues of SEQ ID NO: 17 and has at least 80% sequence identity to the at least 200 consecutive amino acid residues of SEQ ID NO: 17. In some instances, the Fab heavy chain polypeptide comprises an amino acid sequence according to SEQ ID NO: 17. In some instances, the Fab light chain polypeptide comprises an amino acid sequence that has at least 80% sequence identity to the amino acid sequence according to SEQ ID NO: 16. In some instances, the Fab light chain polypeptide comprises an amino acid sequence of at least 100 consecutive amino acid residues of SEQ ID NO: 16. In some instances, the Fab light chain polypeptide comprises an amino acid sequence of at least 200 consecutive amino acid residues of SEQ ID NO: 16. In some instances, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16. In some instances, the Fab light chain polypeptide comprises an amino acid sequence according to SEQ ID NO: 16. In some instances, the scFv heavy chain variable domain comprises an amino acid sequence that has at least 80% sequence identity to the amino acid sequence according to SEQ ID NO: 8. In some instances, the scFv heavy chain variable domain comprises an amino acid sequence of at least 50 consecutive amino acid residues of SEQ ID NO: 8. In some instances, the scFv heavy chain variable domain comprises an amino acid sequence of at least 100 consecutive amino acid residues of SEQ ID NO: 8. In some instances, the scFv heavy chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some instances, the scFv heavy chain variable domain comprises an amino acid sequence according to SEQ ID NO: 8. In some instances, the scFv light chain variable domain comprises an amino acid sequence that has at least 80% sequence identity to the amino acid sequence according to SEQ ID NO: 7. In some instances, the scFv light chain variable domain comprises an amino acid sequence of at least 50 consecutive amino acid residues of SEQ ID NO: 7. In some instances, the scFv light chain variable domain comprises an amino acid sequence of at least 100 consecutive amino acid residues of SEQ ID NO: 7. In some instances, the scFv light chain variable domain comprises an amino acid sequence of at least 100 consecutive amino acid residues of SEQ ID NO: 7 and has at least 80% sequence identity to the at least 100 consecutive amino acid residues of SEQ ID NO: 7. In some instances, the scFv light chain variable domain comprises an amino acid sequence according to SEQ ID NO: 7. In some instances, the scFv comprises an amino acid sequence that has at least 80% sequence identity to the amino acid sequence according to SEQ ID NO: 9. In some instances, the scFv comprises an amino acid sequence of at least 150 consecutive amino acid residues of SEQ ID NO: 9. In some instances, the scFv comprises an amino acid sequence of at least 225 consecutive amino acid residues of SEQ ID NO: 9. In some instances, the scFv comprises an amino acid sequence of at least 225 consecutive amino acid residues of SEQ ID NO: 9 and has at least 80% sequence identity to the at least 225 consecutive amino acid residues of SEQ ID NO: 9. In some instances, the scFv comprises an amino acid sequence according to SEQ ID NO: 9. In some instances, the linker connects the C-terminus of A to an N-terminus of B. In some instances, the linker connects the N-terminus of A to a C-terminus of B. In some instances, the linker connects the C-terminus of A to the N-terminus of the Fab heavy chain polypeptide. In some instances, the linker connects the N-terminus of A to the C-terminus of the Fab heavy chain polypeptide. In some instances, the linker connects the C-terminus of A to the N-terminus of the Fab light chain polypeptide. In some instances, the linker connects the N-terminus of A to the C-terminus of the Fab light chain polypeptide. In some instances, the linker connects the Fab light chain polypeptide to the scFv light chain variable domain. In some instances, the linker connects the Fab light chain polypeptide to the scFv heavy chain variable domain. In some instances, the linker connects the Fab heavy chain polypeptide to the scFv light chain variable domain. In some instances, the linker connects the Fab heavy chain polypeptide to the scFv heavy chain variable domain. In some instances, the linker connects the Fab light chain polypeptide to the N-terminus of the scFv light chain variable domain. In some instances, the linker connects the Fab light chain polypeptide to the C-terminus of the scFv light chain variable domain. In some instances, the linker connects the Fab light chain polypeptide to the N-terminus of the scFv heavy chain variable domain. In some instances, the linker connects the Fab light chain polypeptide to the C-terminus of the scFv heavy chain variable domain. In some instances, the linker connects the Fab heavy chain polypeptide to the N-terminus of the scFv light chain variable domain. In some instances, the linker connects the Fab heavy chain polypeptide to the C-terminus of the scFv light chain variable domain. In some instances, the linker connects the Fab heavy chain polypeptide to the N-terminus of the scFv heavy chain variable domain. In some instances, the linker connects the Fab heavy chain polypeptide to the C-terminus of the scFv heavy chain variable domain. In some instances, the linker is at least 5 amino acids in length. In some instances, the linker is no more than 30 amino acids in length. In some instances, the linker is at least 5 amino acids and no more than 30 amino acids in length. In some instances, the linker is 5 amino acids in length. In some instances, the linker is 15 amino acids in length. In some instances, the linker comprises an amino acid sequence of SEQ ID NO: 19 (GGGGSGGGGSGGGGS) or SEQ ID NO: 18 (GGGGS). In some instances, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some instances, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv light chain variable domain comprises an amino acid sequence of at least 400 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21. In some instances, the linker connects the Fab heavy chain polypeptide to the C-terminus of the scFv light chain variable domain and wherein the Fab light chain polypeptide cosmprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some instances, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some instances, the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 23, and an amino acid sequence of the Fab light chain polypeptide that is connected to the C-terminus of the scFv light chain variable domain comprises an amino acid sequence of at least 400 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22. In some instances, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 34. In some instances, the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 35, and an amino acid sequence of the Fab light chain polypeptide that is connected to the C-terminus of the scFv light chain variable domain comprises an amino acid sequence of at least 400 consecutive amino acid residues of SEQ ID NO: 34. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35, and an amino acid sequence of the Fab light chain polypeptide that is connected to the C-terminus of the scFv light chain variable domain comprises an amino acid sequence of at least 450 consecutive amino acid residues of SEQ ID NO: 34. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35 and has at least 80% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 80% sequence identity to the at least 450 consecutive amino acid residues of SEQ ID NO: 34.

Disclosed herein, in certain embodiments, are isolated polypeptide complexes according to the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D. In some instances, the Fab or Fab′ comprises a Fab light chain polypeptide chain comprising a Fab light chain variable domain and a Fab heavy chain polypeptide comprising a Fab heavy chain variable domain The isolated polypeptide complex according to claim 65 or 66, wherein the scFv comprises a scFv light chain variable domain and a scFv heavy chain variable domain. In some instances, the linker connects the C-terminus of A to the N-terminus of the Fab heavy chain polypeptide. In some instances, the linker connects the C-terminus of A to the N-terminus of the Fab light chain polypeptide. In some instances, the linker connects the C-terminus of the scFv light chain variable domain to the N-terminus of the Fab heavy chain polypeptide. In some instances, the linker connects the C-terminus of scFv light chain variable domain to the N-terminus of the Fab light chain polypeptide. In some instances, the linker connects the C-terminus of the scFv heavy chain variable domain to the N-terminus of the Fab heavy chain polypeptide. In some instances, the linker connects the C-terminus of scFv heavy chain variable domain to the N-terminus of the Fab light chain polypeptide. In some instances, the Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3. In some instances, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3. In some instances, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3. In some instances, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3. In some instances, the Fab heavy chain polypeptide comprises an amino acid sequence that has at least 80% sequence identity to the amino acid sequence according to SEQ ID NO: 17. In some instances, the Fab heavy chain polypeptide comprises an amino acid sequence of at least 100 consecutive amino acid residues of SEQ ID NO: 17. In some instances, the Fab heavy chain polypeptide comprises an amino acid sequence of at least 200 consecutive amino acid residues of SEQ ID NO: 17. In some instances, the Fab heavy chain polypeptide comprises an amino acid sequence of at least 200 consecutive amino acid residues of SEQ ID NO: 17 and has at least 80% sequence identity to the at least 200 consecutive amino acid residues of SEQ ID NO: 17. In some instances, the Fab heavy chain polypeptide comprises an amino acid sequence according to SEQ ID NO: 17. In some instances, the Fab light chain polypeptide comprises an amino acid sequence that has at least 80% sequence identity to the amino acid sequence according to SEQ ID NO: 16. In some instances, the Fab light chain polypeptide comprises an amino acid sequence of at least 100 consecutive amino acid residues of SEQ ID NO: 16. In some instances, the Fab light chain polypeptide comprises an amino acid sequence of at least 200 consecutive amino acid residues of SEQ ID NO: 16. In some instances, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16. In some instances, the Fab light chain polypeptide comprises an amino acid sequence according to SEQ ID NO: 16. In some instances, the scFv heavy chain variable domain comprises an amino acid sequence that has at least 80% sequence identity to the amino acid sequence according to SEQ ID NO: 8. In some instances, the scFv heavy chain variable domain comprises an amino acid sequence of at least 50 consecutive amino acid residues of SEQ ID NO: 8. In some instances, the scFv heavy chain variable domain comprises an amino acid sequence of at least 100 consecutive amino acid residues of SEQ ID NO: 8. In some instances, the scFv heavy chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some instances, the scFv heavy chain variable domain comprises an amino acid sequence according to SEQ ID NO: 8. In some instances, the scFv light chain variable domain comprises an amino acid sequence that has at least 80% sequence identity to the amino acid sequence according to SEQ ID NO: 7. In some instances, the scFv light chain variable domain comprises an amino acid sequence of at least 50 consecutive amino acid residues of SEQ ID NO: 7. In some instances, the scFv light chain variable domain comprises an amino acid sequence of at least 100 consecutive amino acid residues of SEQ ID NO: 7. In some instances, the scFv light chain variable domain comprises an amino acid sequence of at least 100 consecutive amino acid residues of SEQ ID NO: 7 and has at least 80% sequence identity to the at least 100 consecutive amino acid residues of SEQ ID NO: 7. In some instances, the scFv light chain variable domain comprises an amino acid sequence according to SEQ ID NO: 7. In some instances, the scFv comprises an amino acid sequence that has at least 80% sequence identity to the amino acid sequence according to SEQ ID NO: 9. In some instances, the scFv comprises an amino acid sequence of at least 150 consecutive amino acid residues of SEQ ID NO: 9. In some instances, the scFv comprises an amino acid sequence of at least 225 consecutive amino acid residues of SEQ ID NO: 9. In some instances, the scFv comprises an amino acid sequence of at least 225 consecutive amino acid residues of SEQ ID NO: 9 and has at least 80% sequence identity to the at least 225 consecutive amino acid residues of SEQ ID NO: 9. In some instances, the scFv comprises an amino acid sequence according to SEQ ID NO: 9. In some instances, the linker is at least 5 amino acids in length. The isolated polypeptide complex according to claim 103, wherein the linker is no more than 30 amino acids in length. The isolated polypeptide complex according to claim 104, wherein the linker is at least 5 amino acids and no more than 30 amino acids in length. The isolated polypeptide complex according to claim 105, wherein the linker is 5 amino acids in length. The isolated polypeptide complex according to claim 105, wherein the linker is 15 amino acids in length. The isolated polypeptide complex according to claim 105, wherein the linker comprises an amino acid sequence of SEQ ID NO: 19 (GGGGSGGGGSGGGGS) or SEQ ID NO: 18 (GGGGS). In some instances, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some instances, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv light chain variable domain comprises an amino acid sequence of at least 400 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some instances, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some instances, the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 23, and an amino acid sequence of the Fab light chain polypeptide that is connected to the C-terminus of the scFv light chain variable domain comprises an amino acid sequence of at least 400 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22. In some instances, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 34. In some instances, the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 35, and an amino acid sequence of the Fab light chain polypeptide that is connected to the C-terminus of the scFv light chain variable domain comprises an amino acid sequence of at least 400 consecutive amino acid residues of SEQ ID NO: 34. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35, and an amino acid sequence of the Fab light chain polypeptide that is connected to the C-terminus of the scFv light chain variable domain comprises an amino acid sequence of at least 450 consecutive amino acid residues of SEQ ID NO: 34. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35 and has at least 80% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 80% sequence identity to the at least 450 consecutive amino acid residues of SEQ ID NO: 34.

Disclosed herein, in certain embodiments, are pharmaceutical compositions comprising: the isolated polypeptide complex described herein; and a pharmaceutically acceptable excipient.

Disclosed herein, in certain embodiments, are isolated recombinant nucleic acid molecules encoding a polypeptide of the polypeptide complex as described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B illustrate exemplary configurations of an antibody that selectively binds to TROP2 and CD3. FIG. 1A exemplifies a Fab that binds to TROP2 and a scFv that binds to CD3 in which the N-terminus of the Fab heavy chain polypeptide is connected by a linker to the C-terminus of the scFv light chain variable domain. FIG. 1B exemplifies a Fab that binds to TROP2 and a scFv that binds to CD3 in which the N-terminus of the Fab light chain polypeptide is connected by a linker to the C-terminus scFv light chain variable domain.

FIGS. 2A-2B illustrate binding of Ab-3 to TROP2-biotin (FIG. 2A) and CD3E-biotin (FIG. 2B) measured by ELISA.

FIG. 3 illustrates titration data for TROP2 binding of Ab-3.

FIG. 4 illustrates titration data for CD3E binding Ab-3.

FIG. 5 illustrates cytotoxicity of Ab-3 as assessed in a cell viability assay in BxPC3, NCI-N87, Colo205, and HCT116 cells.

FIGS. 6A-6B illustrate polypeptide complex mediated H292 tumor cell killing in the presence of CD8+ T cells.

FIGS. 7A-7B illustrate polypeptide complex mediated HCT116 tumor cell killing in the presence of CD8+ T cells.

FIG. 8 illustrates polypeptide complex mediated MDAMB231 tumor cell killing in the presence of CD8+ T cells.

FIG. 9 illustrates polypeptide pharmacokinetics in cynomolgus monkeys after a single IV bolus injection.

FIG. 10 illustrates cytokine release in cynomolgus monkeys after single IV bolus of TCE.

FIG. 11 illustrates serum liver enzymes in cynomolgus monkeys after single IV bolus of TCE.

FIGS. 12A-12B illustrate titration data for TROP2 binding of Ab-1 and Ab-2.

FIGS. 13A-13B illustrate binding of Ab-1 and Ab-2 to TROP2-biotin (FIG. 13A) and CD3E-biotin (FIG. 13B) measured by ELISA.

FIG. 14 illustrates a graph of mean tumor volume (y-axis) versus days (x-axis) demonstrating in vivo anti-tumor efficacy of polypeptide Ab-1 in an MDA-MB-231 mouse xenograft model of triple negative breast cancer (TNBC).

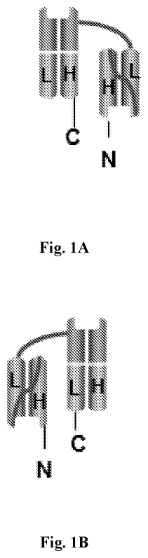

### DETAILED DESCRIPTION

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity.

One such tumor-associated antigen is TROP2. TROP2 (also known as tumor-associated calcium signal transducer 2 or epithelial glycoprotein-1) is the protein product of the TACSTD2 gene, and is a transmembrane glycoprotein that functions in variety of cell signaling pathways, many of which are associated with tumorigenesis. TROP2 has been shown to be overexpressed in multiple human carcinomas, including lung, breast, cervical, ovarian, colorectal, pancreatic, and gastric cancers, and its expression has been correlated with poor patient prognosis. Furthermore, TROP2 functions as an oncogene capable of driving both tumorigenesis and metastasis in epithelial cancers such as colorectal cancer. TROP2 expression in cancer cells has long been correlated with drug resistance, and high levels of TROP2 expression have been shown to correlate with poor prognosis in a variety of cancer types. In a meta-analysis, including data from approximately 2,500 patients, increased TROP2 expression was associated with poor overall survival and disease-free survival outcomes across several solid tumors.

Disclosed herein are antibodies that selectively bind to TROP2 and CD3, in which the anti-TROP2 domain is in a Fab or Fab′ antibody format that is linked to a single-chain variable fragment (scFv) that binds to CD3. The bispecific antibody format of a Fab or Fab′ linked to a scFv provides efficacy and safety advantages over other bispecific antibody formats. In some embodiments, the Fab or Fab′ comprises a Fab light chain polypeptide comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3. In some embodiments, the scFv that binds to CD3 is linked to an N-terminus of the Fab or Fab′ that binds to TROP2.

In some embodiments, the polypeptides or polypeptide complexes described herein are used in a method of treating cancer. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric. In some embodiments, the polypeptides or polypeptide complexes described herein are used in a method of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer. In some embodiments, the polypeptides or polypeptide complexes described herein are used in a method of treating breast cancer, lung cancer, urothelial cancer, endometrial cancer, ovarian cancer, prostate cancer, pancreatic cancer, gastric cancer, colon cancer, head and neck cancer, and glioma.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Phickthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Disclosed herein are isolated polypeptide complexes according to the following formula:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B. Disclosed herein are isolated polypeptide complexes comprising the following formula:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B. Disclosed herein are isolated polypeptide complexes comprising the following formula:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B. Disclosed herein are isolated polypeptide complexes according to the following formula:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B.

Disclosed herein are isolated polypeptide complexes according to the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D. Disclosed herein are isolated polypeptide complexes comprising the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D. Disclosed herein are isolated polypeptide complexes comprising the following formula:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D. Disclosed herein are isolated polypeptide complexes according to the following formula:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D.
Single Chain Variable Fragments (scFv) that Bind to CD3

In some embodiments, the scFv that binds to CD3 comprises a scFv light chain variable domain and a scFv heavy chain variable domain. In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity). In some embodiments, the scFv light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity); and the scFv light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 27; HC-CDR2: SEQ ID NO: 28; HC-CDR3: SEQ ID NO: 29, and wherein the CDRs comprise from 0-2 amino acid modifications in at least one of the HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 24; LC-CDR2: SEQ ID NO: 25; and LC-CDR3: SEQ ID NO: 26, and wherein the CDRs comprise from 0-2 amino acid modifications in at least one of the LC-CDR1, LC-CDR2, or LC-CDR3.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 27; HC-CDR2: SEQ ID NO: 28; HC-CDR3: SEQ ID NO: 29, and wherein the CDRs comprise from 0-2 amino acid modifications in at least one of the HC-CDR1, HC-CDR2, or HC-CDR3; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 24; LC-CDR2: SEQ ID NO: 25; and LC-CDR3: SEQ ID NO: 26, and wherein the CDRs comprise from 0-2 amino acid modifications in at least one of the LC-CDR1, LC-CDR2, or LC-CDR3.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1; HC-CDR2: SEQ ID NO: 2; HC-CDR3: SEQ ID NO: 3. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 27; HC-CDR2: SEQ ID NO: 28; HC-CDR3: SEQ ID NO: 29. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 24; LC-CDR2: SEQ ID NO: 25; and LC-CDR3: SEQ ID NO: 26.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 27; HC-CDR2: SEQ ID NO: 28; HC-CDR3: SEQ ID NO: 29; and the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 24; LC-CDR2: SEQ ID NO: 25; and LC-CDR3: SEQ ID NO: 26.

TABLE 1
anti-CD3 scFv heavy chain variable domain
complementarity determining regions (CDR)s
(as based on the IMGT CDR numbering system).

| | Amino Acid | SEQ |
|---|---|---|
| Construct | Sequence | ID |
| Description | (N to C) | NO: |
| CD3: HC: CDR1 | GFTFNKYA | 1 |
| CD3: HC: CDR2 | IRSKYNNYAT | 2 |
| CD3: HC: CDR3 | VRHGNFGNSYISYWAY | 3 |

TABLE 2
anti-CD3 scFv light chain variable domain
complementarity determining regions (CDR)s

| | Amino Acid | SEQ |
|---|---|---|
| Construct | Sequence | ID |
| Description | (N to C) | NO: |
| CD3: LC: CDR1 | TGAVTSGNY | 4 |
| CD3: LC: CDR2 | GTK | 5 |
| CD3: LC: CDR3 | VLWYSNRWV | 6 |

In some embodiments, the scFv heavy chain variable domain comprises an amino acid sequence that has at least 70% sequence identity to the amino acid sequence according to SEQ ID NO: 8. In some embodiments, the scFv heavy chain variable domain comprises an amino acid sequence that has at least 80% sequence identity to the amino acid sequence according to SEQ ID NO: 8. In some embodiments, the scFv heavy chain variable domain comprises an amino acid sequence that has at least 85% sequence identity to the amino acid sequence according to SEQ ID NO: 8. In some embodiments, the scFv heavy chain variable domain comprises an amino acid sequence that has at least 90% sequence identity to the amino acid sequence according to SEQ ID NO: 8. In some embodiments, the scFv heavy chain variable domain comprises an amino acid sequence that has at least 91% sequence identity to the amino acid sequence according to SEQ ID NO: 8. In some embodiments, the scFv heavy chain variable domain comprises an amino acid sequence that has at least 92% sequence identity to the amino acid sequence according to SEQ ID NO: 8. In some embodiments, the scFv heavy chain variable domain comprises an amino acid sequence that has at least 93% sequence identity to the amino acid sequence according to SEQ ID NO: 8. In some embodiments, the scFv heavy chain variable domain comprises an amino acid sequence that has at least 94% sequence identity to the amino acid sequence according to SEQ ID NO: 8. In some embodiments, the scFv heavy chain variable domain comprises an amino acid sequence that has at least 95% sequence identity to the amino acid sequence according to SEQ ID NO: 8. In some embodiments, the scFv heavy chain variable domain comprises an amino acid sequence that has at least 96% sequence identity to the amino acid sequence according to SEQ ID NO: 8. In some embodiments, the scFv heavy chain variable domain comprises an amino acid sequence that has at least 97% sequence identity to the amino acid sequence according to SEQ ID NO: 8. In some embodiments, the scFv heavy chain variable domain comprises an amino acid sequence that has at least 98% sequence identity to the amino acid sequence according to SEQ ID NO: 8. In some embodiments, the scFv heavy chain variable domain comprises an amino acid sequence that has at least 99% sequence identity to the amino acid sequence according to SEQ ID NO: 8. In some embodiments, the scFv heavy chain variable domain comprises an amino acid sequence according to SEQ ID NO: 8.

In some embodiments, the scFv heavy chain variable domain comprises an amino acid sequence of at least 50 consecutive amino acid residues of SEQ ID NO: 8. In some embodiments, the scFv heavy chain variable domain comprises an amino acid sequence of at least 60 consecutive amino acid residues of SEQ ID NO: 8. In some embodiments, the scFv heavy chain variable domain comprises an amino acid sequence of at least 70 consecutive amino acid residues of SEQ ID NO: 8. In some embodiments, the scFv heavy chain variable domain comprises an amino acid sequence of at least 80 consecutive amino acid residues of SEQ ID NO: 8. In some embodiments, the scFv heavy chain variable domain comprises an amino acid sequence of at least 90 consecutive amino acid residues of SEQ ID NO: 8. In some embodiments, the scFv heavy chain variable domain comprises an amino acid sequence of at least 100 consecutive amino acid residues of SEQ ID NO: 8. In some embodiments, the scFv heavy chain variable domain comprises an amino acid sequence of at least 105 consecutive amino acid residues of SEQ ID NO: 8. In some embodiments, the scFv heavy chain variable domain comprises an amino acid sequence of at least 110 consecutive amino acid residues of SEQ ID NO: 8. In some embodiments, the scFv heavy chain variable domain comprises an amino acid sequence of at least 115 consecutive amino acid residues of SEQ ID NO: 8. In some embodiments, the scFv heavy chain variable domain comprises an amino acid sequence of at least 120 consecutive amino acid residues of SEQ ID NO: 8.

In some embodiments, the scFv heavy chain variable domain comprises an amino acid sequence of at least 50 consecutive amino acid residues of SEQ ID NO: 8, and has at least 80% sequence identity to the at least 50 consecutive amino acid residues of SEQ ID NO: 8. In some embodiments, the scFv heavy chain variable domain comprises an amino acid sequence of at least 60 consecutive amino acid residues of SEQ ID NO: 8, and has at least 80% sequence identity to the at least 60 consecutive amino acid residues of SEQ ID NO: 8. In some embodiments, the scFv heavy chain variable domain comprises an amino acid sequence of at least 70 consecutive amino acid residues of SEQ ID NO: 8, and has at least 80% sequence identity to the at least 70 consecutive amino acid residues of SEQ ID NO: 8. In some embodiments, the scFv heavy chain variable domain comprises an amino acid sequence of at least 80 consecutive amino acid residues of SEQ ID NO: 8, and has at least 80% sequence identity to the at least 80 consecutive amino acid residues of SEQ ID NO: 8. In some embodiments, the scFv heavy chain variable domain comprises an amino acid sequence of at least 90 consecutive amino acid residues of SEQ ID NO: 8, and has at least 80% sequence identity to the at least 90 consecutive amino acid residues of SEQ ID NO: 8. In some embodiments, the scFv heavy chain variable domain comprises an amino acid sequence of at least 100 consecutive amino acid residues of SEQ ID NO: 8, and has at least 80% sequence identity to the at least 100 consecutive amino acid residues of SEQ ID NO: 8. In some embodiments, the scFv heavy chain variable domain comprises an amino acid sequence of at least 105 consecutive amino acid residues of SEQ ID NO: 8, and has at least 80% sequence identity to the at least 105 consecutive amino acid residues of SEQ ID NO: 8. In some embodiments, the scFv heavy chain variable domain comprises an amino acid sequence of at least 110 consecutive amino acid residues of SEQ ID NO: 8, and has at least 80% sequence identity to the at least 110 consecutive amino acid residues of SEQ ID NO: 8. In some embodiments, the scFv heavy chain variable domain comprises an amino acid sequence of at least 115 consecutive amino acid residues of SEQ ID NO: 8, and has at least 80% sequence identity to the at least 115 consecutive amino acid residues of SEQ ID NO: 8. In some embodiments, the scFv heavy chain variable domain comprises an amino acid sequence of at least 120 consecutive amino acid residues of SEQ ID NO: 8, and has at least 80% sequence identity to the at least 120 consecutive amino acid residues of SEQ ID NO: 8.

In some embodiments, the scFv heavy chain variable domain comprises an amino acid sequence of at least 50 consecutive amino acid residues of SEQ ID NO: 8, and has at least 90% sequence identity to the at least 50 consecutive amino acid residues of SEQ ID NO: 8. In some embodiments, the scFv heavy chain variable domain comprises an amino acid sequence of at least 60 consecutive amino acid residues of SEQ ID NO: 8, and has at least 90% sequence identity to the at least 60 consecutive amino acid residues of SEQ ID NO: 8. In some embodiments, the scFv heavy chain variable domain comprises an amino acid sequence of at least 70 consecutive amino acid residues of SEQ ID NO: 8, and has at least 90% sequence identity to the at least 70 consecutive amino acid residues of SEQ ID NO: 8. In some embodiments, the scFv heavy chain variable domain comprises an amino acid sequence of at least 80 consecutive amino acid residues of SEQ ID NO: 8, and has at least 90% sequence identity to the at least 80 consecutive amino acid residues of SEQ ID NO: 8. In some embodiments, the scFv heavy chain variable domain comprises an amino acid sequence of at least 90 consecutive amino acid residues of SEQ ID NO: 8, and has at least 90% sequence identity to the at least 90 consecutive amino acid residues of SEQ ID NO: 8. In some embodiments, the scFv heavy chain variable domain comprises an amino acid sequence of at least 100 consecutive amino acid residues of SEQ ID NO: 8, and has at least 90% sequence identity to the at least 100 consecutive amino acid residues of SEQ ID NO: 8. In some embodiments, the scFv heavy chain variable domain comprises an amino acid sequence of at least 105 consecutive amino acid residues of SEQ ID NO: 8, and has at least 90% sequence identity to the at least 105 consecutive amino acid residues of SEQ ID NO: 8. In some embodiments, the scFv heavy chain variable domain comprises an amino acid sequence of at least 110 consecutive amino acid residues of SEQ ID NO: 8, and has at least 90% sequence identity to the at least 110 consecutive amino acid residues of SEQ ID NO: 8. In some embodiments, the scFv heavy chain variable domain comprises an amino acid sequence of at least 115 consecutive amino acid residues of SEQ ID NO: 8, and has at least 90% sequence identity to the at least 115 consecutive amino acid residues of SEQ ID NO: 8. In some embodiments, the scFv heavy chain variable domain comprises an amino acid sequence of at least 120 consecutive amino acid residues of SEQ ID NO: 8, and has at least 90% sequence identity to the at least 120 consecutive amino acid residues of SEQ ID NO: 8.

In some embodiments, the scFv heavy chain variable domain comprises an amino acid sequence of at least 50 consecutive amino acid residues of SEQ ID NO: 8, and has at least 95% sequence identity to the at least 50 consecutive amino acid residues of SEQ ID NO: 8. In some embodiments, the scFv heavy chain variable domain comprises an amino acid sequence of at least 60 consecutive amino acid residues of SEQ ID NO: 8, and has at least 95% sequence identity to the at least 60 consecutive amino acid residues of SEQ ID NO: 8. In some embodiments, the scFv heavy chain variable domain comprises an amino acid sequence of at least 70 consecutive amino acid residues of SEQ ID NO: 8, and has at least 95% sequence identity to the at least 70 consecutive amino acid residues of SEQ ID NO: 8. In some embodiments, the scFv heavy chain variable domain comprises an amino acid sequence of at least 80 consecutive amino acid residues of SEQ ID NO: 8, and has at least 95% sequence identity to the at least 80 consecutive amino acid residues of SEQ ID NO: 8. In some embodiments, the scFv heavy chain variable domain comprises an amino acid sequence of at least 90 consecutive amino acid residues of SEQ ID NO: 8, and has at least 95% sequence identity to the at least 90 consecutive amino acid residues of SEQ ID NO: 8. In some embodiments, the scFv heavy chain variable domain comprises an amino acid sequence of at least 100 consecutive amino acid residues of SEQ ID NO: 8, and has at least 95% sequence identity to the at least 100 consecutive amino acid residues of SEQ ID NO: 8. In some embodiments, the scFv heavy chain variable domain comprises an amino acid sequence of at least 105 consecutive amino acid residues of SEQ ID NO: 8, and has at least 95% sequence identity to the at least 105 consecutive amino acid residues of SEQ ID NO: 8. In some embodiments, the scFv heavy chain variable domain comprises an amino acid sequence of at least 110 consecutive amino acid residues of SEQ ID NO: 8, and has at least 95% sequence identity to the at least 110 consecutive amino acid residues of SEQ ID NO: 8. In some embodiments, the scFv heavy chain variable domain comprises an amino acid sequence of at least 115 consecutive amino acid residues of SEQ ID NO: 8, and has at least 95% sequence identity to the at least 115 consecutive amino acid residues of SEQ ID NO: 8. In some embodiments, the scFv heavy chain variable domain comprises an amino acid sequence of at least 120 consecutive amino acid residues of SEQ ID NO: 8, and has at least 95% sequence identity to the at least 120 consecutive amino acid residues of SEQ ID NO: 8.

In some embodiments, the scFv heavy chain variable domain comprises an amino acid sequence of at least 100 consecutive amino acid residues of SEQ ID NO: 8, and has at least 99% sequence identity to the at least 100 consecutive amino acid residues of SEQ ID NO: 8. In some embodiments, the scFv heavy chain variable domain comprises an amino acid sequence of at least 105 consecutive amino acid residues of SEQ ID NO: 8, and has at least 99% sequence identity to the at least 105 consecutive amino acid residues of SEQ ID NO: 8. In some embodiments, the scFv heavy chain variable domain comprises an amino acid sequence of at least 110 consecutive amino acid residues of SEQ ID NO: 8, and has at least 99% sequence identity to the at least 110 consecutive amino acid residues of SEQ ID NO: 8. In some embodiments, the scFv heavy chain variable domain comprises an amino acid sequence of at least 115 consecutive amino acid residues of SEQ ID NO: 8, and has at least 99% sequence identity to the at least 115 consecutive amino acid residues of SEQ ID NO: 8. In some embodiments, the scFv heavy chain variable domain comprises an amino acid sequence of at least 120 consecutive amino acid residues of SEQ ID NO: 8, and has at least 99% sequence identity to the at least 120 consecutive amino acid residues of SEQ ID NO: 8.

In some embodiments, the scFv heavy chain variable domain comprises an amino acid sequence that has at least 70% sequence identity to the amino acid sequence according to SEQ ID NO: 31. In some embodiments, the scFv heavy chain variable domain comprises an amino acid sequence that has at least 80% sequence identity to the amino acid sequence according to SEQ ID NO: 31. In some embodiments, the scFv heavy chain variable domain comprises an amino acid sequence that has at least 85% sequence identity to the amino acid sequence according to SEQ ID NO: 31. In some embodiments, the scFv heavy chain variable domain comprises an amino acid sequence that has at least 90% sequence identity to the amino acid sequence according to SEQ ID NO: 31. In some embodiments, the scFv heavy chain variable domain comprises an amino acid sequence that has at least 91% sequence identity to the amino acid sequence according to SEQ ID NO: 31. In some embodiments, the scFv heavy chain variable domain comprises an amino acid sequence that has at least 92% sequence identity to the amino acid sequence according to SEQ ID NO: 31. In some embodiments, the scFv heavy chain variable domain comprises an amino acid sequence that has at least 93% sequence identity to the amino acid sequence according to SEQ ID NO: 31. In some embodiments, the scFv heavy chain variable domain comprises an amino acid sequence that has at least 94% sequence identity to the amino acid sequence according to SEQ ID NO: 31. In some embodiments, the scFv heavy chain variable domain comprises an amino acid sequence that has at least 95% sequence identity to the amino acid sequence according to SEQ ID NO: 31. In some embodiments, the scFv heavy chain variable domain comprises an amino acid sequence that has at least 96% sequence identity to the amino acid sequence according to SEQ ID NO: 31. In some embodiments, the scFv heavy chain variable domain comprises an amino acid sequence that has at least 97% sequence identity to the amino acid sequence according to SEQ ID NO: 31. In some embodiments, the scFv heavy chain variable domain comprises an amino acid sequence that has at least 98% sequence identity to the amino acid sequence according to SEQ ID NO: 31. In some embodiments, the scFv heavy chain variable domain comprises an amino acid sequence that has at least 99% sequence identity to the amino acid sequence according to SEQ ID NO: 31. In some embodiments, the scFv heavy chain variable domain comprises an amino acid sequence according to SEQ ID NO: 31.

In some embodiments, the scFv heavy chain variable domain comprises an amino acid sequence of at least 50 consecutive amino acid residues of SEQ ID NO: 31. In some embodiments, the scFv heavy chain variable domain comprises an amino acid sequence of at least 60 consecutive amino acid residues of SEQ ID NO: 31. In some embodiments, the scFv heavy chain variable domain comprises an amino acid sequence of at least 70 consecutive amino acid residues of SEQ ID NO: 31. In some embodiments, the scFv heavy chain variable domain comprises an amino acid sequence of at least 80 consecutive amino acid residues of SEQ ID NO: 31. In some embodiments, the scFv heavy chain variable domain comprises an amino acid sequence of at least 90 consecutive amino acid residues of SEQ ID NO: 31. In some embodiments, the scFv heavy chain variable domain comprises an amino acid sequence of at least 100 consecutive amino acid residues of SEQ ID NO: 31. In some embodiments, the scFv heavy chain variable domain comprises an amino acid sequence of at least 105 consecutive amino acid residues of SEQ ID NO: 31. In some embodiments, the scFv heavy chain variable domain comprises an amino acid sequence of at least 110 consecutive amino acid residues of SEQ ID NO: 31. In some embodiments, the scFv heavy chain variable domain comprises an amino acid sequence of at least 115 consecutive amino acid residues of SEQ ID NO: 31. In some embodiments, the scFv heavy chain variable domain comprises an amino acid sequence of at least 120 consecutive amino acid residues of SEQ ID NO: 31.

In some embodiments, the scFv heavy chain variable domain comprises an amino acid sequence of at least 50 consecutive amino acid residues of SEQ ID NO: 31, and has at least 80% sequence identity to the at least 50 consecutive amino acid residues of SEQ ID NO: 31. In some embodiments, the scFv heavy chain variable domain comprises an amino acid sequence of at least 60 consecutive amino acid residues of SEQ ID NO: 31, and has at least 80% sequence identity to the at least 60 consecutive amino acid residues of SEQ ID NO: 31. In some embodiments, the scFv heavy chain variable domain comprises an amino acid sequence of at least 70 consecutive amino acid residues of SEQ ID NO: 31, and has at least 80% sequence identity to the at least 70 consecutive amino acid residues of SEQ ID NO: 31. In some embodiments, the scFv heavy chain variable domain comprises an amino acid sequence of at least 80 consecutive amino acid residues of SEQ ID NO: 31, and has at least 80% sequence identity to the at least 80 consecutive amino acid residues of SEQ ID NO: 31. In some embodiments, the scFv heavy chain variable domain comprises an amino acid sequence of at least 90 consecutive amino acid residues of SEQ ID NO: 31, and has at least 80% sequence identity to the at least 90 consecutive amino acid residues of SEQ ID NO: 31. In some embodiments, the scFv heavy chain variable domain comprises an amino acid sequence of at least 100 consecutive amino acid residues of SEQ ID NO: 31, and has at least 80% sequence identity to the at least 100 consecutive amino acid residues of SEQ ID NO: 31. In some embodiments, the scFv heavy chain variable domain comprises an amino acid sequence of at least 105 consecutive amino acid residues of SEQ ID NO: 31, and has at least 80% sequence identity to the at least 105 consecutive amino acid residues of SEQ ID NO: 31. In some embodiments, the scFv heavy chain variable domain comprises an amino acid sequence of at least 110 consecutive amino acid residues of SEQ ID NO: 31, and has at least 80% sequence identity to the at least 110 consecutive amino acid residues of SEQ ID NO: 31. In some embodiments, the scFv heavy chain variable domain comprises an amino acid sequence of at least 115 consecutive amino acid residues of SEQ ID NO: 31, and has at least 80% sequence identity to the at least 115 consecutive amino acid residues of SEQ ID NO: 31. In some embodiments, the scFv heavy chain variable domain comprises an amino acid sequence of at least 120 consecutive amino acid residues of SEQ ID NO: 31, and has at least 80% sequence identity to the at least 120 consecutive amino acid residues of SEQ ID NO: 31.

In some embodiments, the scFv heavy chain variable domain comprises an amino acid sequence of at least 50 consecutive amino acid residues of SEQ ID NO: 31, and has at least 90% sequence identity to the at least 50 consecutive amino acid residues of SEQ ID NO: 31. In some embodiments, the scFv heavy chain variable domain comprises an amino acid sequence of at least 60 consecutive amino acid residues of SEQ ID NO: 31, and has at least 90% sequence identity to the at least 60 consecutive amino acid residues of SEQ ID NO: 31. In some embodiments, the scFv heavy chain variable domain comprises an amino acid sequence of at least 70 consecutive amino acid residues of SEQ ID NO: 31, and has at least 90% sequence identity to the at least 70 consecutive amino acid residues of SEQ ID NO: 31. In some embodiments, the scFv heavy chain variable domain comprises an amino acid sequence of at least 80 consecutive amino acid residues of SEQ ID NO: 31, and has at least 90% sequence identity to the at least 80 consecutive amino acid residues of SEQ ID NO: 31. In some embodiments, the scFv heavy chain variable domain comprises an amino acid sequence of at least 90 consecutive amino acid residues of SEQ ID NO: 31, and has at least 90% sequence identity to the at least 90 consecutive amino acid residues of SEQ ID NO: 31. In some embodiments, the scFv heavy chain variable domain comprises an amino acid sequence of at least 100 consecutive amino acid residues of SEQ ID NO: 31, and has at least 90% sequence identity to the at least 100 consecutive amino acid residues of SEQ ID NO: 31. In some embodiments, the scFv heavy chain variable domain comprises an amino acid sequence of at least 105 consecutive amino acid residues of SEQ ID NO: 31, and has at least 90% sequence identity to the at least 105 consecutive amino acid residues of SEQ ID NO: 31. In some embodiments, the scFv heavy chain variable domain comprises an amino acid sequence of at least 110 consecutive amino acid residues of SEQ ID NO: 31, and has at least 90% sequence identity to the at least 110 consecutive amino acid residues of SEQ ID NO: 31. In some embodiments, the scFv heavy chain variable domain comprises an amino acid sequence of at least 115 consecutive amino acid residues of SEQ ID NO: 31, and has at least 90% sequence identity to the at least 115 consecutive amino acid residues of SEQ ID NO: 31. In some embodiments, the scFv heavy chain variable domain comprises an amino acid sequence of at least 120 consecutive amino acid residues of SEQ ID NO: 31, and has at least 90% sequence identity to the at least 120 consecutive amino acid residues of SEQ ID NO: 31.

In some embodiments, the scFv heavy chain variable domain comprises an amino acid sequence of at least 50 consecutive amino acid residues of SEQ ID NO: 31, and has at least 95% sequence identity to the at least 50 consecutive amino acid residues of SEQ ID NO: 31. In some embodiments, the scFv heavy chain variable domain comprises an amino acid sequence of at least 60 consecutive amino acid residues of SEQ ID NO: 31, and has at least 95% sequence identity to the at least 60 consecutive amino acid residues of SEQ ID NO: 31. In some embodiments, the scFv heavy chain variable domain comprises an amino acid sequence of at least 70 consecutive amino acid residues of SEQ ID NO: 31, and has at least 95% sequence identity to the at least 70 consecutive amino acid residues of SEQ ID NO: 31. In some embodiments, the scFv heavy chain variable domain comprises an amino acid sequence of at least 80 consecutive amino acid residues of SEQ ID NO: 31, and has at least 95% sequence identity to the at least 80 consecutive amino acid residues of SEQ ID NO: 31. In some embodiments, the scFv heavy chain variable domain comprises an amino acid sequence of at least 90 consecutive amino acid residues of SEQ ID NO: 31, and has at least 95% sequence identity to the at least 90 consecutive amino acid residues of SEQ ID NO: 31. In some embodiments, the scFv heavy chain variable domain comprises an amino acid sequence of at least 100 consecutive amino acid residues of SEQ ID NO: 31, and has at least 95% sequence identity to the at least 100 consecutive amino acid residues of SEQ ID NO: 31. In some embodiments, the scFv heavy chain variable domain comprises an amino acid sequence of at least 105 consecutive amino acid residues of SEQ ID NO: 31, and has at least 95% sequence identity to the at least 105 consecutive amino acid residues of SEQ ID NO: 31. In some embodiments, the scFv heavy chain variable domain comprises an amino acid sequence of at least 110 consecutive amino acid residues of SEQ ID NO: 31, and has at least 95% sequence identity to the at least 110 consecutive amino acid residues of SEQ ID NO: 31. In some embodiments, the scFv heavy chain variable domain comprises an amino acid sequence of at least 115 consecutive amino acid residues of SEQ ID NO: 31, and has at least 95% sequence identity to the at least 115 consecutive amino acid residues of SEQ ID NO: 31. In some embodiments, the scFv heavy chain variable domain comprises an amino acid sequence of at least 120 consecutive amino acid residues of SEQ ID NO: 31, and has at least 95% sequence identity to the at least 120 consecutive amino acid residues of SEQ ID NO: 31.

In some embodiments, the scFv heavy chain variable domain comprises an amino acid sequence of at least 100 consecutive amino acid residues of SEQ ID NO: 31, and has at least 99% sequence identity to the at least 100 consecutive amino acid residues of SEQ ID NO: 31. In some embodiments, the scFv heavy chain variable domain comprises an amino acid sequence of at least 105 consecutive amino acid residues of SEQ ID NO: 31, and has at least 99% sequence identity to the at least 105 consecutive amino acid residues of SEQ ID NO: 31. In some embodiments, the scFv heavy chain variable domain comprises an amino acid sequence of at least 110 consecutive amino acid residues of SEQ ID NO: 31, and has at least 99% sequence identity to the at least 110 consecutive amino acid residues of SEQ ID NO: 31. In some embodiments, the scFv heavy chain variable domain comprises an amino acid sequence of at least 115 consecutive amino acid residues of SEQ ID NO: 31, and has at least 99% sequence identity to the at least 115 consecutive amino acid residues of SEQ ID NO: 31. In some embodiments, the scFv heavy chain variable domain comprises an amino acid sequence of at least 120 consecutive amino acid residues of SEQ ID NO: 31, and has at least 99% sequence identity to the at least 120 consecutive amino acid residues of SEQ ID NO: 31.

In some embodiments, the scFv light chain variable domain comprises an amino acid sequence that has at least 70% sequence identity to the amino acid sequence according to SEQ ID NO: 7. In some embodiments, the scFv light chain variable domain comprises an amino acid sequence that has at least 80% sequence identity to the amino acid sequence according to SEQ ID NO: 7. In some embodiments, the scFv light chain variable domain comprises an amino acid sequence that has at least 85% sequence identity to the amino acid sequence according to SEQ ID NO: 7. In some embodiments, the scFv light chain variable domain comprises an amino acid sequence that has at least 90% sequence identity to the amino acid sequence according to SEQ ID NO: 7. In some embodiments, the scFv light chain variable domain comprises an amino acid sequence that has at least 91% sequence identity to the amino acid sequence according to SEQ ID NO: 7. In some embodiments, the scFv light chain variable domain comprises an amino acid sequence that has at least 92% sequence identity to the amino acid sequence according to SEQ ID NO: 7. In some embodiments, the scFv light chain variable domain comprises an amino acid sequence that has at least 93% sequence identity to the amino acid sequence according to SEQ ID NO: 7. In some embodiments, the scFv light chain variable domain comprises an amino acid sequence that has at least 94% sequence identity to the amino acid sequence according to SEQ ID NO: 7. In some embodiments, the scFv light chain variable domain comprises an amino acid sequence that has at least 95% sequence identity to the amino acid sequence according to SEQ ID NO: 7. In some embodiments, the scFv light chain variable domain comprises an amino acid sequence that has at least 96% sequence identity to the amino acid sequence according to SEQ ID NO: 7. In some embodiments, the scFv light chain variable domain comprises an amino acid sequence that has at least 97% sequence identity to the amino acid sequence according to SEQ ID NO: 7. In some embodiments, the scFv light chain variable domain comprises an amino acid sequence that has at least 98% sequence identity to the amino acid sequence according to SEQ ID NO: 7. In some embodiments, the scFv light chain variable domain comprises an amino acid sequence that has at least 99% sequence identity to the amino acid sequence according to SEQ ID NO: 7. In some embodiments, the scFv light chain variable domain comprises an amino acid sequence according to SEQ ID NO: 7.

In some embodiments, the scFv light chain variable domain comprises an amino acid sequence of at least 50 consecutive amino acid residues of SEQ ID NO: 7. In some embodiments, the scFv light chain variable domain comprises an amino acid sequence of at least 60 consecutive amino acid residues of SEQ ID NO: 7. In some embodiments, the scFv light chain variable domain comprises an amino acid sequence of at least 70 consecutive amino acid residues of SEQ ID NO: 7. In some embodiments, the scFv light chain variable domain comprises an amino acid sequence of at least 80 consecutive amino acid residues of SEQ ID NO: 7. In some embodiments, the scFv light chain variable domain comprises an amino acid sequence of at least 90 consecutive amino acid residues of SEQ ID NO: 7. In some embodiments, the scFv light chain variable domain comprises an amino acid sequence of at least 100 consecutive amino acid residues of SEQ ID NO: 7. In some embodiments, the scFv light chain variable domain comprises an amino acid sequence of at least 105 consecutive amino acid residues of SEQ ID NO: 7.

In some embodiments, the scFv light chain variable domain comprises an amino acid sequence of at least 50 consecutive amino acid residues of SEQ ID NO: 7, and has at least 80% sequence identity to the at least 50 consecutive amino acid residues of SEQ ID NO: 7. In some embodiments, the scFv light chain variable domain comprises an amino acid sequence of at least 60 consecutive amino acid residues of SEQ ID NO: 7, and has at least 80% sequence identity to the at least 60 consecutive amino acid residues of SEQ ID NO: 7. In some embodiments, the scFv light chain variable domain comprises an amino acid sequence of at least 70 consecutive amino acid residues of SEQ ID NO: 7, and has at least 80% sequence identity to the at least 70 consecutive amino acid residues of SEQ ID NO: 7. In some embodiments, the scFv light chain variable domain comprises an amino acid sequence of at least 80 consecutive amino acid residues of SEQ ID NO: 7, and has at least 80% sequence identity to the at least 80 consecutive amino acid residues of SEQ ID NO: 7. In some embodiments, the scFv light chain variable domain comprises an amino acid sequence of at least 90 consecutive amino acid residues of SEQ ID NO: 7, and has at least 80% sequence identity to the at least 90 consecutive amino acid residues of SEQ ID NO: 7. In some embodiments, the scFv light chain variable domain comprises an amino acid sequence of at least 100 consecutive amino acid residues of SEQ ID NO: 7, and has at least 80% sequence identity to the at least 100 consecutive amino acid residues of SEQ ID NO: 7. In some embodiments, the scFv light chain variable domain comprises an amino acid sequence of at least 105 consecutive amino acid residues of SEQ ID NO: 7, and has at least 80% sequence identity to the at least 105 consecutive amino acid residues of SEQ ID NO: 7.

In some embodiments, the scFv light chain variable domain comprises an amino acid sequence of at least 50 consecutive amino acid residues of SEQ ID NO: 7, and has at least 90% sequence identity to the at least 50 consecutive amino acid residues of SEQ ID NO: 7. In some embodiments, the scFv light chain variable domain comprises an amino acid sequence of at least 60 consecutive amino acid residues of SEQ ID NO: 7, and has at least 90% sequence identity to the at least 60 consecutive amino acid residues of SEQ ID NO: 7. In some embodiments, the scFv light chain variable domain comprises an amino acid sequence of at least 70 consecutive amino acid residues of SEQ ID NO: 7, and has at least 90% sequence identity to the at least 70 consecutive amino acid residues of SEQ ID NO: 7. In some embodiments, the scFv light chain variable domain comprises an amino acid sequence of at least 80 consecutive amino acid residues of SEQ ID NO: 7, and has at least 90% sequence identity to the at least 80 consecutive amino acid residues of SEQ ID NO: 7. In some embodiments, the scFv light chain variable domain comprises an amino acid sequence of at least 90 consecutive amino acid residues of SEQ ID NO: 7, and has at least 90% sequence identity to the at least 90 consecutive amino acid residues of SEQ ID NO: 7. In some embodiments, the scFv light chain variable domain comprises an amino acid sequence of at least 100 consecutive amino acid residues of SEQ ID NO: 7, and has at least 90% sequence identity to the at least 100 consecutive amino acid residues of SEQ ID NO: 7. In some embodiments, the scFv light chain variable domain comprises an amino acid sequence of at least 105 consecutive amino acid residues of SEQ ID NO: 7, and has at least 90% sequence identity to the at least 105 consecutive amino acid residues of SEQ ID NO: 7.

In some embodiments, the scFv light chain variable domain comprises an amino acid sequence of at least 50 consecutive amino acid residues of SEQ ID NO: 7, and has at least 95% sequence identity to the at least 50 consecutive amino acid residues of SEQ ID NO: 7. In some embodiments, the scFv light chain variable domain comprises an amino acid sequence of at least 60 consecutive amino acid residues of SEQ ID NO: 7, and has at least 95% sequence identity to the at least 60 consecutive amino acid residues of SEQ ID NO: 7. In some embodiments, the scFv light chain variable domain comprises an amino acid sequence of at least 70 consecutive amino acid residues of SEQ ID NO: 7, and has at least 95% sequence identity to the at least 70 consecutive amino acid residues of SEQ ID NO: 7. In some embodiments, the scFv light chain variable domain comprises an amino acid sequence of at least 80 consecutive amino acid residues of SEQ ID NO: 7, and has at least 95% sequence identity to the at least 80 consecutive amino acid residues of SEQ ID NO: 7. In some embodiments, the scFv light chain variable domain comprises an amino acid sequence of at least 90 consecutive amino acid residues of SEQ ID NO: 7, and has at least 95% sequence identity to the at least 90 consecutive amino acid residues of SEQ ID NO: 7. In some embodiments, the scFv light chain variable domain comprises an amino acid sequence of at least 100 consecutive amino acid residues of SEQ ID NO: 7, and has at least 95% sequence identity to the at least 100 consecutive amino acid residues of SEQ ID NO: 7. In some embodiments, the scFv light chain variable domain comprises an amino acid sequence of at least 105 consecutive amino acid residues of SEQ ID NO: 7, and has at least 95% sequence identity to the at least 105 consecutive amino acid residues of SEQ ID NO: 7. In some embodiments, the scFv light chain variable domain comprises an amino acid sequence of at least 100 consecutive amino acid residues of SEQ ID NO: 7, and has at least 99% sequence identity to the at least 100 consecutive amino acid residues of SEQ ID NO: 7. In some embodiments, the scFv light chain variable domain comprises an amino acid sequence of at least 105 consecutive amino acid residues of SEQ ID NO: 7, and has at least 99% sequence identity to the at least 105 consecutive amino acid residues of SEQ ID NO: 7.

In some embodiments, the scFv light chain variable domain comprises an amino acid sequence that has at least 70% sequence identity to the amino acid sequence according to SEQ ID NO: 30. In some embodiments, the scFv light chain variable domain comprises an amino acid sequence that has at least 80% sequence identity to the amino acid sequence according to SEQ ID NO: 30. In some embodiments, the scFv light chain variable domain comprises an amino acid sequence that has at least 85% sequence identity to the amino acid sequence according to SEQ ID NO: 30. In some embodiments, the scFv light chain variable domain comprises an amino acid sequence that has at least 90% sequence identity to the amino acid sequence according to SEQ ID NO: 30. In some embodiments, the scFv light chain variable domain comprises an amino acid sequence that has at least 91% sequence identity to the amino acid sequence according to SEQ ID NO: 30. In some embodiments, the scFv light chain variable domain comprises an amino acid sequence that has at least 92% sequence identity to the amino acid sequence according to SEQ ID NO: 30. In some embodiments, the scFv light chain variable domain comprises an amino acid sequence that has at least 93% sequence identity to the amino acid sequence according to SEQ ID NO: 30. In some embodiments, the scFv light chain variable domain comprises an amino acid sequence that has at least 94% sequence identity to the amino acid sequence according to SEQ ID NO: 30. In some embodiments, the scFv light chain variable domain comprises an amino acid sequence that has at least 95% sequence identity to the amino acid sequence according to SEQ ID NO: 30. In some embodiments, the scFv light chain variable domain comprises an amino acid sequence that has at least 96% sequence identity to the amino acid sequence according to SEQ ID NO: 30. In some embodiments, the scFv light chain variable domain comprises an amino acid sequence that has at least 97% sequence identity to the amino acid sequence according to SEQ ID NO: 30. In some embodiments, the scFv light chain variable domain comprises an amino acid sequence that has at least 98% sequence identity to the amino acid sequence according to SEQ ID NO: 30. In some embodiments, the scFv light chain variable domain comprises an amino acid sequence that has at least 99% sequence identity to the amino acid sequence according to SEQ ID NO: 30. In some embodiments, the scFv light chain variable domain comprises an amino acid sequence according to SEQ ID NO: 30.

In some embodiments, the scFv light chain variable domain comprises an amino acid sequence of at least 50 consecutive amino acid residues of SEQ ID NO: 30. In some embodiments, the scFv light chain variable domain comprises an amino acid sequence of at least 60 consecutive amino acid residues of SEQ ID NO: 30. In some embodiments, the scFv light chain variable domain comprises an amino acid sequence of at least 70 consecutive amino acid residues of SEQ ID NO: 30. In some embodiments, the scFv light chain variable domain comprises an amino acid sequence of at least 80 consecutive amino acid residues of SEQ ID NO: 30. In some embodiments, the scFv light chain variable domain comprises an amino acid sequence of at least 90 consecutive amino acid residues of SEQ ID NO: 30. In some embodiments, the scFv light chain variable domain comprises an amino acid sequence of at least 100 consecutive amino acid residues of SEQ ID NO: 30. In some embodiments, the scFv light chain variable domain comprises an amino acid sequence of at least 105 consecutive amino acid residues of SEQ ID NO: 30.

In some embodiments, the scFv light chain variable domain comprises an amino acid sequence of at least 50 consecutive amino acid residues of SEQ ID NO: 30, and has at least 80% sequence identity to the at least 50 consecutive amino acid residues of SEQ ID NO: 30. In some embodiments, the scFv light chain variable domain comprises an amino acid sequence of at least 60 consecutive amino acid residues of SEQ ID NO: 30, and has at least 80% sequence identity to the at least 60 consecutive amino acid residues of SEQ ID NO: 30. In some embodiments, the scFv light chain variable domain comprises an amino acid sequence of at least 70 consecutive amino acid residues of SEQ ID NO: 30, and has at least 80% sequence identity to the at least 70 consecutive amino acid residues of SEQ ID NO: 30. In some embodiments, the scFv light chain variable domain comprises an amino acid sequence of at least 80 consecutive amino acid residues of SEQ ID NO: 30, and has at least 80% sequence identity to the at least 80 consecutive amino acid residues of SEQ ID NO: 30. In some embodiments, the scFv light chain variable domain comprises an amino acid sequence of at least 90 consecutive amino acid residues of SEQ ID NO: 30, and has at least 80% sequence identity to the at least 90 consecutive amino acid residues of SEQ ID NO: 30. In some embodiments, the scFv light chain variable domain comprises an amino acid sequence of at least 100 consecutive amino acid residues of SEQ ID NO: 30, and has at least 80% sequence identity to the at least 100 consecutive amino acid residues of SEQ ID NO: 30. In some embodiments, the scFv light chain variable domain comprises an amino acid sequence of at least 105 consecutive amino acid residues of SEQ ID NO: 30, and has at least 80% sequence identity to the at least 105 consecutive amino acid residues of SEQ ID NO: 30.

In some embodiments, the scFv light chain variable domain comprises an amino acid sequence of at least 50 consecutive amino acid residues of SEQ ID NO: 30, and has at least 90% sequence identity to the at least 50 consecutive amino acid residues of SEQ ID NO: 30. In some embodiments, the scFv light chain variable domain comprises an amino acid sequence of at least 60 consecutive amino acid residues of SEQ ID NO: 30, and has at least 90% sequence identity to the at least 60 consecutive amino acid residues of SEQ ID NO: 30. In some embodiments, the scFv light chain variable domain comprises an amino acid sequence of at least 70 consecutive amino acid residues of SEQ ID NO: 30, and has at least 90% sequence identity to the at least 70 consecutive amino acid residues of SEQ ID NO: 30. In some embodiments, the scFv light chain variable domain comprises an amino acid sequence of at least 80 consecutive amino acid residues of SEQ ID NO: 30, and has at least 90% sequence identity to the at least 80 consecutive amino acid residues of SEQ ID NO: 30. In some embodiments, the scFv light chain variable domain comprises an amino acid sequence of at least 90 consecutive amino acid residues of SEQ ID NO: 30, and has at least 90% sequence identity to the at least 90 consecutive amino acid residues of SEQ ID NO: 30. In some embodiments, the scFv light chain variable domain comprises an amino acid sequence of at least 100 consecutive amino acid residues of SEQ ID NO: 30, and has at least 90% sequence identity to the at least 100 consecutive amino acid residues of SEQ ID NO: 30. In some embodiments, the scFv light chain variable domain comprises an amino acid sequence of at least 105 consecutive amino acid residues of SEQ ID NO: 30, and has at least 90% sequence identity to the at least 105 consecutive amino acid residues of SEQ ID NO: 30.

In some embodiments, the scFv light chain variable domain comprises an amino acid sequence of at least 50 consecutive amino acid residues of SEQ ID NO: 30, and has at least 95% sequence identity to the at least 50 consecutive amino acid residues of SEQ ID NO: 30. In some embodiments, the scFv light chain variable domain comprises an amino acid sequence of at least 60 consecutive amino acid residues of SEQ ID NO: 30, and has at least 95% sequence identity to the at least 60 consecutive amino acid residues of SEQ ID NO: 30. In some embodiments, the scFv light chain variable domain comprises an amino acid sequence of at least 70 consecutive amino acid residues of SEQ ID NO: 30, and has at least 95% sequence identity to the at least 70 consecutive amino acid residues of SEQ ID NO: 30. In some embodiments, the scFv light chain variable domain comprises an amino acid sequence of at least 80 consecutive amino acid residues of SEQ ID NO: 30, and has at least 95% sequence identity to the at least 80 consecutive amino acid residues of SEQ ID NO: 30. In some embodiments, the scFv light chain variable domain comprises an amino acid sequence of at least 90 consecutive amino acid residues of SEQ ID NO: 30, and has at least 95% sequence identity to the at least 90 consecutive amino acid residues of SEQ ID NO: 30. In some embodiments, the scFv light chain variable domain comprises an amino acid sequence of at least 100 consecutive amino acid residues of SEQ ID NO: 30, and has at least 95% sequence identity to the at least 100 consecutive amino acid residues of SEQ ID NO: 30. In some embodiments, the scFv light chain variable domain comprises an amino acid sequence of at least 105 consecutive amino acid residues of SEQ ID NO: 30, and has at least 95% sequence identity to the at least 105 consecutive amino acid residues of SEQ ID NO: 30. In some embodiments, the scFv light chain variable domain comprises an amino acid sequence of at least 100 consecutive amino acid residues of SEQ ID NO: 30, and has at least 99% sequence identity to the at least 100 consecutive amino acid residues of SEQ ID NO: 30. In some embodiments, the scFv light chain variable domain comprises an amino acid sequence of at least 105 consecutive amino acid residues of SEQ ID NO: 30, and has at least 99% sequence identity to the at least 105 consecutive amino acid residues of SEQ ID NO: 30.

In some embodiments, the scFv heavy chain variable domain comprises an amino acid sequence that has at least 70% sequence identity to the amino acid sequence according to SEQ ID NO: 8; and the scFv light chain variable domain comprises an amino acid sequence that has at least 70% sequence identity to the amino acid sequence according to SEQ ID NO: 7. In some embodiments, the scFv heavy chain variable domain comprises an amino acid sequence that has at least 80% sequence identity to the amino acid sequence according to SEQ ID NO: 8; and the scFv light chain variable domain comprises an amino acid sequence that has at least 80% sequence identity to the amino acid sequence according to SEQ ID NO: 7. In some embodiments, the scFv heavy chain variable domain comprises an amino acid sequence that has at least 85% sequence identity to the amino acid sequence according to SEQ ID NO: 8; and the scFv light chain variable domain comprises an amino acid sequence that has at least 85% sequence identity to the amino acid sequence according to SEQ ID NO: 7. In some embodiments, the scFv heavy chain variable domain comprises an amino acid sequence that has at least 90% sequence identity to the amino acid sequence according to SEQ ID NO: 8; and the scFv light chain variable domain comprises an amino acid sequence that has at least 90% sequence identity to the amino acid sequence according to SEQ ID NO: 7. In some embodiments, the scFv heavy chain variable domain comprises an amino acid sequence that has at least 91% sequence identity to the amino acid sequence according to SEQ ID NO: 8; and the scFv light chain variable domain comprises an amino acid sequence that has at least 91% sequence identity to the amino acid sequence according to SEQ ID NO: 7. In some embodiments, the scFv heavy chain variable domain comprises an amino acid sequence that has at least 92% sequence identity to the amino acid sequence according to SEQ ID NO: 8; and the scFv light chain variable domain comprises an amino acid sequence that has at least 92% sequence identity to the amino acid sequence according to SEQ ID NO: 7. In some embodiments, the scFv heavy chain variable domain comprises an amino acid sequence that has at least 93% sequence identity to the amino acid sequence according to SEQ ID NO: 8; and the scFv light chain variable domain comprises an amino acid sequence that has at least 93% sequence identity to the amino acid sequence according to SEQ ID NO: 7. In some embodiments, the scFv heavy chain variable domain comprises an amino acid sequence that has at least 94% sequence identity to the amino acid sequence according to SEQ ID NO: 8; and the scFv light chain variable domain comprises an amino acid sequence that has at least 94% sequence identity to the amino acid sequence according to SEQ ID NO: 7. In some embodiments, the scFv heavy chain variable domain comprises an amino acid sequence that has at least 95% sequence identity to the amino acid sequence according to SEQ ID NO: 8; and the scFv light chain variable domain comprises an amino acid sequence that has at least 95% sequence identity to the amino acid sequence according to SEQ ID NO: 7. In some embodiments, the scFv heavy chain variable domain comprises an amino acid sequence that has at least 96% sequence identity to the amino acid sequence according to SEQ ID NO: 8; and the scFv light chain variable domain comprises an amino acid sequence that has at least 96% sequence identity to the amino acid sequence according to SEQ ID NO: 7. In some embodiments, the scFv heavy chain variable domain comprises an amino acid sequence that has at least 97% sequence identity to the amino acid sequence according to SEQ ID NO: 8; and the scFv light chain variable domain comprises an amino acid sequence that has at least 97% sequence identity to the amino acid sequence according to SEQ ID NO: 7. In some embodiments, the scFv heavy chain variable domain comprises an amino acid sequence that has at least 98% sequence identity to the amino acid sequence according to SEQ ID NO: 8; and the scFv light chain variable domain comprises an amino acid sequence that has at least 98% sequence identity to the amino acid sequence according to SEQ ID NO: 7. In some embodiments, the scFv heavy chain variable domain comprises an amino acid sequence that has at least 99% sequence identity to the amino acid sequence according to SEQ ID NO: 8; and the scFv light chain variable domain comprises an amino acid sequence that has at least 99% sequence identity to the amino acid sequence according to SEQ ID NO: 7.

In some embodiments, the scFv heavy chain variable domain comprises an amino acid sequence that has at least 70% sequence identity to the amino acid sequence according to SEQ ID NO: 31; and the scFv light chain variable domain comprises an amino acid sequence that has at least 70% sequence identity to the amino acid sequence according to SEQ ID NO: 30. In some embodiments, the scFv heavy chain variable domain comprises an amino acid sequence that has at least 80% sequence identity to the amino acid sequence according to SEQ ID NO: 31; and the scFv light chain variable domain comprises an amino acid sequence that has at least 80% sequence identity to the amino acid sequence according to SEQ ID NO: 30. In some embodiments, the scFv heavy chain variable domain comprises an amino acid sequence that has at least 85% sequence identity to the amino acid sequence according to SEQ ID NO: 31; and the scFv light chain variable domain comprises an amino acid sequence that has at least 85% sequence identity to the amino acid sequence according to SEQ ID NO: 30. In some embodiments, the scFv heavy chain variable domain comprises an amino acid sequence that has at least 90% sequence identity to the amino acid sequence according to SEQ ID NO: 31; and the scFv light chain variable domain comprises an amino acid sequence that has at least 90% sequence identity to the amino acid sequence according to SEQ ID NO: 30. In some embodiments, the scFv heavy chain variable domain comprises an amino acid sequence that has at least 91% sequence identity to the amino acid sequence according to SEQ ID NO: 31; and the scFv light chain variable domain comprises an amino acid sequence that has at least 91% sequence identity to the amino acid sequence according to SEQ ID NO: 30. In some embodiments, the scFv heavy chain variable domain comprises an amino acid sequence that has at least 92% sequence identity to the amino acid sequence according to SEQ ID NO: 31; and the scFv light chain variable domain comprises an amino acid sequence that has at least 92% sequence identity to the amino acid sequence according to SEQ ID NO: 30. In some embodiments, the scFv heavy chain variable domain comprises an amino acid sequence that has at least 93% sequence identity to the amino acid sequence according to SEQ ID NO: 31; and the scFv light chain variable domain comprises an amino acid sequence that has at least 93% sequence identity to the amino acid sequence according to SEQ ID NO: 30. In some embodiments, the scFv heavy chain variable domain comprises an amino acid sequence that has at least 94% sequence identity to the amino acid sequence according to SEQ ID NO: 31; and the scFv light chain variable domain comprises an amino acid sequence that has at least 94% sequence identity to the amino acid sequence according to SEQ ID NO: 30. In some embodiments, the scFv heavy chain variable domain comprises an amino acid sequence that has at least 95% sequence identity to the amino acid sequence according to SEQ ID NO: 31; and the scFv light chain variable domain comprises an amino acid sequence that has at least 95% sequence identity to the amino acid sequence according to SEQ ID NO: 30. In some embodiments, the scFv heavy chain variable domain comprises an amino acid sequence that has at least 96% sequence identity to the amino acid sequence according to SEQ ID NO: 31; and the scFv light chain variable domain comprises an amino acid sequence that has at least 96% sequence identity to the amino acid sequence according to SEQ ID NO: 30. In some embodiments, the scFv heavy chain variable domain comprises an amino acid sequence that has at least 97% sequence identity to the amino acid sequence according to SEQ ID NO: 31; and the scFv light chain variable domain comprises an amino acid sequence that has at least 97% sequence identity to the amino acid sequence according to SEQ ID NO: 30. In some embodiments, the scFv heavy chain variable domain comprises an amino acid sequence that has at least 98% sequence identity to the amino acid sequence according to SEQ ID NO: 31; and the scFv light chain variable domain comprises an amino acid sequence that has at least 98% sequence identity to the amino acid sequence according to SEQ ID NO: 30. In some embodiments, the scFv heavy chain variable domain comprises an amino acid sequence that has at least 99% sequence identity to the amino acid sequence according to SEQ ID NO: 31; and the scFv light chain variable domain comprises an amino acid sequence that has at least 99% sequence identity to the amino acid sequence according to SEQ ID NO: 30.

In some embodiments, the scFv comprises an amino acid sequence that has at least 70% sequence identity to the amino acid sequence according to SEQ ID NO: 9. In some embodiments, the scFv comprises an amino acid sequence that has at least 80% sequence identity to the amino acid sequence according to SEQ ID NO: 9. In some embodiments, the scFv comprises an amino acid sequence that has at least 85% sequence identity to the amino acid sequence according to SEQ ID NO: 9. In some embodiments, the scFv comprises an amino acid sequence that has at least 90% sequence identity to the amino acid sequence according to SEQ ID NO: 9. In some embodiments, the scFv comprises an amino acid sequence that has at least 91% sequence identity to the amino acid sequence according to SEQ ID NO: 9. In some embodiments, the scFv comprises an amino acid sequence that has at least 92% sequence identity to the amino acid sequence according to SEQ ID NO: 9. In some embodiments, the scFv comprises an amino acid sequence that has at least 93% sequence identity to the amino acid sequence according to SEQ ID NO: 9. In some embodiments, the scFv comprises an amino acid sequence that has at least 94% sequence identity to the amino acid sequence according to SEQ ID NO: 9. In some embodiments, the scFv comprises an amino acid sequence that has at least 95% sequence identity to the amino acid sequence according to SEQ ID NO: 9. In some embodiments, the scFv comprises an amino acid sequence that has at least 96% sequence identity to the amino acid sequence according to SEQ ID NO: 9. In some embodiments, the scFv comprises an amino acid sequence that has at least 97% sequence identity to the amino acid sequence according to SEQ ID NO: 9. In some embodiments, the scFv comprises an amino acid sequence that has at least 98% sequence identity to the amino acid sequence according to SEQ ID NO: 9. In some embodiments, the scFv comprises an amino acid sequence that has at least 99% sequence identity to the amino acid sequence according to SEQ ID NO: 9. In some embodiments, the scFv comprises an amino acid sequence according to SEQ ID NO: 9.

In some embodiments, the scFv comprises an amino acid sequence of at least 150 consecutive amino acid residues of SEQ ID NO: 9. In some embodiments, the scFv comprises an amino acid sequence of at least 175 consecutive amino acid residues of SEQ ID NO: 9. In some embodiments, the scFv comprises an amino acid sequence of at least 200 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 220 consecutive amino acid residues of SEQ ID NO: 9. In some embodiments, the scFv comprises an amino acid sequence of at least 225 consecutive amino acid residues of SEQ ID NO: 9. In some embodiments, the scFv comprises an amino acid sequence of at least 230 consecutive amino acid residues of SEQ ID NO: 9. In some embodiments, the scFv comprises an amino acid sequence of at least 235 consecutive amino acid residues of SEQ ID NO: 9. In some embodiments, the scFv comprises an amino acid sequence of at least 240 consecutive amino acid residues of SEQ ID NO: 9.

In some embodiments, the scFv comprises an amino acid sequence of at least 150 consecutive amino acid residues of SEQ ID NO: 9, and has at least 80% sequence identity to the at least 150 consecutive amino acid residues of SEQ ID NO: 9. In some embodiments, the scFv comprises an amino acid sequence of at least 175 consecutive amino acid residues of SEQ ID NO: 9, and has at least 80% sequence identity to the at least 175 consecutive amino acid residues of SEQ ID NO: 9. In some embodiments, the scFv comprises an amino acid sequence of at least 200 consecutive amino acid residues of SEQ ID NO: 9, and has at least 80% sequence identity to the at least 200 consecutive amino acid residues of SEQ ID NO: 9. In some embodiments, the scFv comprises an amino acid sequence of at least 210 consecutive amino acid residues of SEQ ID NO: 9, and has at least 80% sequence identity to the at least 210 consecutive amino acid residues of SEQ ID NO: 9. In some embodiments, the scFv comprises an amino acid sequence of at least 220 consecutive amino acid residues of SEQ ID NO: 9, and has at least 80% sequence identity to the at least 220 consecutive amino acid residues of SEQ ID NO: 9. In some embodiments, the scFv comprises an amino acid sequence of at least 225 consecutive amino acid residues of SEQ ID NO: 9, and has at least 80% sequence identity to the at least 225 consecutive amino acid residues of SEQ ID NO: 9. In some embodiments, the scFv comprises an amino acid sequence of at least 230 consecutive amino acid residues of SEQ ID NO: 9, and has at least 80% sequence identity to the at least 230 consecutive amino acid residues of SEQ ID NO: 9. In some embodiments, the scFv comprises an amino acid sequence of at least 235 consecutive amino acid residues of SEQ ID NO: 9, and has at least 80% sequence identity to the at least 235 consecutive amino acid residues of SEQ ID NO: 9. In some embodiments, the scFv comprises an amino acid sequence of at least 240 consecutive amino acid residues of SEQ ID NO: 9, and has at least 80% sequence identity to the at least 240 consecutive amino acid residues of SEQ ID NO: 9.

In some embodiments, the scFv comprises an amino acid sequence of at least 150 consecutive amino acid residues of SEQ ID NO: 9, and has at least 90% sequence identity to the at least 150 consecutive amino acid residues of SEQ ID NO: 9. In some embodiments, the scFv comprises an amino acid sequence of at least 175 consecutive amino acid residues of SEQ ID NO: 9, and has at least 90% sequence identity to the at least 175 consecutive amino acid residues of SEQ ID NO: 9. In some embodiments, the scFv comprises an amino acid sequence of at least 200 consecutive amino acid residues of SEQ ID NO: 9, and has at least 90% sequence identity to the at least 200 consecutive amino acid residues of SEQ ID NO: 9. In some embodiments, the scFv comprises an amino acid sequence of at least 210 consecutive amino acid residues of SEQ ID NO: 9, and has at least 90% sequence identity to the at least 210 consecutive amino acid residues of SEQ ID NO: 9. In some embodiments, the scFv comprises an amino acid sequence of at least 220 consecutive amino acid residues of SEQ ID NO: 9, and has at least 90% sequence identity to the at least 220 consecutive amino acid residues of SEQ ID NO: 9. In some embodiments, the scFv comprises an amino acid sequence of at least 225 consecutive amino acid residues of SEQ ID NO: 9, and has at least 90% sequence identity to the at least 225 consecutive amino acid residues of SEQ ID NO: 9. In some embodiments, the scFv comprises an amino acid sequence of at least 230 consecutive amino acid residues of SEQ ID NO: 9, and has at least 90% sequence identity to the at least 230 consecutive amino acid residues of SEQ ID NO: 9. In some embodiments, the scFv comprises an amino acid sequence of at least 235 consecutive amino acid residues of SEQ ID NO: 9, and has at least 90% sequence identity to the at least 235 consecutive amino acid residues of SEQ ID NO: 9. In some embodiments, the scFv comprises an amino acid sequence of at least 240 consecutive amino acid residues of SEQ ID NO: 9, and has at least 90% sequence identity to the at least 240 consecutive amino acid residues of SEQ ID NO: 9.

In some embodiments, the scFv comprises an amino acid sequence of at least 150 consecutive amino acid residues of SEQ ID NO: 9, and has at least 95% sequence identity to the at least 150 consecutive amino acid residues of SEQ ID NO: 9. In some embodiments, the scFv comprises an amino acid sequence of at least 175 consecutive amino acid residues of SEQ ID NO: 9, and has at least 95% sequence identity to the at least 175 consecutive amino acid residues of SEQ ID NO: 9. In some embodiments, the scFv comprises an amino acid sequence of at least 200 consecutive amino acid residues of SEQ ID NO: 9, and has at least 95% sequence identity to the at least 200 consecutive amino acid residues of SEQ ID NO: 9. In some embodiments, the scFv comprises an amino acid sequence of at least 210 consecutive amino acid residues of SEQ ID NO: 9, and has at least 95% sequence identity to the at least 210 consecutive amino acid residues of SEQ ID NO: 9. In some embodiments, the scFv comprises an amino acid sequence of at least 220 consecutive amino acid residues of SEQ ID NO: 9, and has at least 95% sequence identity to the at least 220 consecutive amino acid residues of SEQ ID NO: 9. In some embodiments, the scFv comprises an amino acid sequence of at least 225 consecutive amino acid residues of SEQ ID NO: 9, and has at least 95% sequence identity to the at least 225 consecutive amino acid residues of SEQ ID NO: 9. In some embodiments, the scFv comprises an amino acid sequence of at least 230 consecutive amino acid residues of SEQ ID NO: 9, and has at least 95% sequence identity to the at least 230 consecutive amino acid residues of SEQ ID NO: 9. In some embodiments, the scFv comprises an amino acid sequence of at least 235 consecutive amino acid residues of SEQ ID NO: 9, and has at least 95% sequence identity to the at least 235 consecutive amino acid residues of SEQ ID NO: 9. In some embodiments, the scFv comprises an amino acid sequence of at least 240 consecutive amino acid residues of SEQ ID NO: 9, and has at least 95% sequence identity to the at least 240 consecutive amino acid residues of SEQ ID NO: 9.

In some embodiments, the scFv comprises an amino acid sequence of at least 150 consecutive amino acid residues of SEQ ID NO: 9, and has at least 99% sequence identity to the at least 150 consecutive amino acid residues of SEQ ID NO: 9. In some embodiments, the scFv comprises an amino acid sequence of at least 175 consecutive amino acid residues of SEQ ID NO: 9, and has at least 99% sequence identity to the at least 175 consecutive amino acid residues of SEQ ID NO: 9. In some embodiments, the scFv comprises an amino acid sequence of at least 200 consecutive amino acid residues of SEQ ID NO: 9, and has at least 99% sequence identity to the at least 200 consecutive amino acid residues of SEQ ID NO: 9. In some embodiments, the scFv comprises an amino acid sequence of at least 210 consecutive amino acid residues of SEQ ID NO: 9, and has at least 99% sequence identity to the at least 210 consecutive amino acid residues of SEQ ID NO: 9. In some embodiments, the scFv comprises an amino acid sequence of at least 220 consecutive amino acid residues of SEQ ID NO: 9, and has at least 99% sequence identity to the at least 220 consecutive amino acid residues of SEQ ID NO: 9. In some embodiments, the scFv comprises an amino acid sequence of at least 225 consecutive amino acid residues of SEQ ID NO: 9, and has at least 99% sequence identity to the at least 225 consecutive amino acid residues of SEQ ID NO: 9. In some embodiments, the scFv comprises an amino acid sequence of at least 230 consecutive amino acid residues of SEQ ID NO: 9, and has at least 99% sequence identity to the at least 230 consecutive amino acid residues of SEQ ID NO: 9. In some embodiments, the scFv comprises an amino acid sequence of at least 235 consecutive amino acid residues of SEQ ID NO: 9, and has at least 99% sequence identity to the at least 235 consecutive amino acid residues of SEQ ID NO: 9. In some embodiments, the scFv comprises an amino acid sequence of at least 240 consecutive amino acid residues of SEQ ID NO: 9, and has at least 99% sequence identity to the at least 240 consecutive amino acid residues of SEQ ID NO: 9.

In some embodiments, the scFv comprises an amino acid sequence that has at least 70% sequence identity to the amino acid sequence according to SEQ ID NO: 32. In some embodiments, the scFv comprises an amino acid sequence that has at least 80% sequence identity to the amino acid sequence according to SEQ ID NO: 32. In some embodiments, the scFv comprises an amino acid sequence that has at least 85% sequence identity to the amino acid sequence according to SEQ ID NO: 32. In some embodiments, the scFv comprises an amino acid sequence that has at least 90% sequence identity to the amino acid sequence according to SEQ ID NO: 32. In some embodiments, the scFv comprises an amino acid sequence that has at least 91% sequence identity to the amino acid sequence according to SEQ ID NO: 32. In some embodiments, the scFv comprises an amino acid sequence that has at least 92% sequence identity to the amino acid sequence according to SEQ ID NO: 32. In some embodiments, the scFv comprises an amino acid sequence that has at least 93% sequence identity to the amino acid sequence according to SEQ ID NO: 32. In some embodiments, the scFv comprises an amino acid sequence that has at least 94% sequence identity to the amino acid sequence according to SEQ ID NO: 32. In some embodiments, the scFv comprises an amino acid sequence that has at least 95% sequence identity to the amino acid sequence according to SEQ ID NO: 32. In some embodiments, the scFv comprises an amino acid sequence that has at least 96% sequence identity to the amino acid sequence according to SEQ ID NO: 32. In some embodiments, the scFv comprises an amino acid sequence that has at least 97% sequence identity to the amino acid sequence according to SEQ ID NO: 32. In some embodiments, the scFv comprises an amino acid sequence that has at least 98% sequence identity to the amino acid sequence according to SEQ ID NO: 32. In some embodiments, the scFv comprises an amino acid sequence that has at least 99% sequence identity to the amino acid sequence according to SEQ ID NO: 32. In some embodiments, the scFv comprises an amino acid sequence according to SEQ ID NO: 32.

In some embodiments, the scFv comprises an amino acid sequence of at least 150 consecutive amino acid residues of SEQ ID NO: 32. In some embodiments, the scFv comprises an amino acid sequence of at least 175 consecutive amino acid residues of SEQ ID NO: 32. In some embodiments, the scFv comprises an amino acid sequence of at least 200 consecutive amino acid residues of SEQ ID NO: 32. In some embodiments, the scFv comprises an amino acid sequence of at least 210 consecutive amino acid residues of SEQ ID NO: 32. In some embodiments, the scFv comprises an amino acid sequence of at least 220 consecutive amino acid residues of SEQ ID NO: 32. In some embodiments, the scFv comprises an amino acid sequence of at least 225 consecutive amino acid residues of SEQ ID NO: 32. In some embodiments, the scFv comprises an amino acid sequence of at least 230 consecutive amino acid residues of SEQ ID NO: 32. In some embodiments, the scFv comprises an amino acid sequence of at least 235 consecutive amino acid residues of SEQ ID NO: 32. In some embodiments, the scFv comprises an amino acid sequence of at least 240 consecutive amino acid residues of SEQ ID NO: 32.

In some embodiments, the scFv comprises an amino acid sequence of at least 150 consecutive amino acid residues of SEQ ID NO: 32, and has at least 80% sequence identity to the at least 150 consecutive amino acid residues of SEQ ID NO: 32. In some embodiments, the scFv comprises an amino acid sequence of at least 175 consecutive amino acid residues of SEQ ID NO: 32, and has at least 80% sequence identity to the at least 175 consecutive amino acid residues of SEQ ID NO: 32. In some embodiments, the scFv comprises an amino acid sequence of at least 200 consecutive amino acid residues of SEQ ID NO: 32, and has at least 80% sequence identity to the at least 200 consecutive amino acid residues of SEQ ID NO: 32. In some embodiments, the scFv comprises an amino acid sequence of at least 210 consecutive amino acid residues of SEQ ID NO: 32, and has at least 80% sequence identity to the at least 210 consecutive amino acid residues of SEQ ID NO: 32. In some embodiments, the scFv comprises an amino acid sequence of at least 220 consecutive amino acid residues of SEQ ID NO: 32, and has at least 80% sequence identity to the at least 220 consecutive amino acid residues of SEQ ID NO: 32. In some embodiments, the scFv comprises an amino acid sequence of at least 225 consecutive amino acid residues of SEQ ID NO: 32, and has at least 80% sequence identity to the at least 225 consecutive amino acid residues of SEQ ID NO: 32. In some embodiments, the scFv comprises an amino acid sequence of at least 230 consecutive amino acid residues of SEQ ID NO: 32, and has at least 80% sequence identity to the at least 230 consecutive amino acid residues of SEQ ID NO: 32. In some embodiments, the scFv comprises an amino acid sequence of at least 235 consecutive amino acid residues of SEQ ID NO: 32, and has at least 80% sequence identity to the at least 235 consecutive amino acid residues of SEQ ID NO: 32. In some embodiments, the scFv comprises an amino acid sequence of at least 240 consecutive amino acid residues of SEQ ID NO: 32, and has at least 80% sequence identity to the at least 240 consecutive amino acid residues of SEQ ID NO: 32.

In some embodiments, the scFv comprises an amino acid sequence of at least 150 consecutive amino acid residues of SEQ ID NO: 32, and has at least 90% sequence identity to the at least 150 consecutive amino acid residues of SEQ ID NO: 32. In some embodiments, the scFv comprises an amino acid sequence of at least 175 consecutive amino acid residues of SEQ ID NO: 32, and has at least 90% sequence identity to the at least 175 consecutive amino acid residues of SEQ ID NO: 32. In some embodiments, the scFv comprises an amino acid sequence of at least 200 consecutive amino acid residues of SEQ ID NO: 32, and has at least 90% sequence identity to the at least 200 consecutive amino acid residues of SEQ ID NO: 32. In some embodiments, the scFv comprises an amino acid sequence of at least 210 consecutive amino acid residues of SEQ ID NO: 32, and has at least 90% sequence identity to the at least 210 consecutive amino acid residues of SEQ ID NO: 32. In some embodiments, the scFv comprises an amino acid sequence of at least 220 consecutive amino acid residues of SEQ ID NO: 32, and has at least 90% sequence identity to the at least 220 consecutive amino acid residues of SEQ ID NO: 32. In some embodiments, the scFv comprises an amino acid sequence of at least 225 consecutive amino acid residues of SEQ ID NO: 32, and has at least 90% sequence identity to the at least 225 consecutive amino acid residues of SEQ ID NO: 32. In some embodiments, the scFv comprises an amino acid sequence of at least 230 consecutive amino acid residues of SEQ ID NO: 32, and has at least 90% sequence identity to the at least 230 consecutive amino acid residues of SEQ ID NO: 32. In some embodiments, the scFv comprises an amino acid sequence of at least 235 consecutive amino acid residues of SEQ ID NO: 32, and has at least 90% sequence identity to the at least 235 consecutive amino acid residues of SEQ ID NO: 32. In some embodiments, the scFv comprises an amino acid sequence of at least 240 consecutive amino acid residues of SEQ ID NO: 32, and has at least 90% sequence identity to the at least 240 consecutive amino acid residues of SEQ ID NO: 32.

In some embodiments, the scFv comprises an amino acid sequence of at least 150 consecutive amino acid residues of SEQ ID NO: 32, and has at least 95% sequence identity to the at least 150 consecutive amino acid residues of SEQ ID NO: 32. In some embodiments, the scFv comprises an amino acid sequence of at least 175 consecutive amino acid residues of SEQ ID NO: 32, and has at least 95% sequence identity to the at least 175 consecutive amino acid residues of SEQ ID NO: 32. In some embodiments, the scFv comprises an amino acid sequence of at least 200 consecutive amino acid residues of SEQ ID NO: 32, and has at least 95% sequence identity to the at least 200 consecutive amino acid residues of SEQ ID NO: 32. In some embodiments, the scFv comprises an amino acid sequence of at least 210 consecutive amino acid residues of SEQ ID NO: 32, and has at least 95% sequence identity to the at least 210 consecutive amino acid residues of SEQ ID NO: 32. In some embodiments, the scFv comprises an amino acid sequence of at least 220 consecutive amino acid residues of SEQ ID NO: 32, and has at least 95% sequence identity to the at least 220 consecutive amino acid residues of SEQ ID NO: 32. In some embodiments, the scFv comprises an amino acid sequence of at least 225 consecutive amino acid residues of SEQ ID NO: 32, and has at least 95% sequence identity to the at least 225 consecutive amino acid residues of SEQ ID NO: 32. In some embodiments, the scFv comprises an amino acid sequence of at least 230 consecutive amino acid residues of SEQ ID NO: 32, and has at least 95% sequence identity to the at least 230 consecutive amino acid residues of SEQ ID NO: 32. In some embodiments, the scFv comprises an amino acid sequence of at least 235 consecutive amino acid residues of SEQ ID NO: 32, and has at least 95% sequence identity to the at least 235 consecutive amino acid residues of SEQ ID NO: 32. In some embodiments, the scFv comprises an amino acid sequence of at least 240 consecutive amino acid residues of SEQ ID NO: 32, and has at least 95% sequence identity to the at least 240 consecutive amino acid residues of SEQ ID NO: 32.

In some embodiments, the scFv comprises an amino acid sequence of at least 150 consecutive amino acid residues of SEQ ID NO: 32, and has at least 99% sequence identity to the at least 150 consecutive amino acid residues of SEQ ID NO: 32. In some embodiments, the scFv comprises an amino acid sequence of at least 175 consecutive amino acid residues of SEQ ID NO: 32, and has at least 99% sequence identity to the at least 175 consecutive amino acid residues of SEQ ID NO: 32. In some embodiments, the scFv comprises an amino acid sequence of at least 200 consecutive amino acid residues of SEQ ID NO: 32, and has at least 99% sequence identity to the at least 200 consecutive amino acid residues of SEQ ID NO: 32. In some embodiments, the scFv comprises an amino acid sequence of at least 210 consecutive amino acid residues of SEQ ID NO: 32, and has at least 99% sequence identity to the at least 210 consecutive amino acid residues of SEQ ID NO: 32. In some embodiments, the scFv comprises an amino acid sequence of at least 220 consecutive amino acid residues of SEQ ID NO: 32, and has at least 99% sequence identity to the at least 220 consecutive amino acid residues of SEQ ID NO: 32. In some embodiments, the scFv comprises an amino acid sequence of at least 225 consecutive amino acid residues of SEQ ID NO: 32, and has at least 99% sequence identity to the at least 225 consecutive amino acid residues of SEQ ID NO: 32. In some embodiments, the scFv comprises an amino acid sequence of at least 230 consecutive amino acid residues of SEQ ID NO: 32, and has at least 99% sequence identity to the at least 230 consecutive amino acid residues of SEQ ID NO: 32. In some embodiments, the scFv comprises an amino acid sequence of at least 235 consecutive amino acid residues of SEQ ID NO: 32, and has at least 99% sequence identity to the at least 235 consecutive amino acid residues of SEQ ID NO: 32. In some embodiments, the scFv comprises an amino acid sequence of at least 240 consecutive amino acid residues of SEQ ID NO: 32, and has at least 99% sequence identity to the at least 240 consecutive amino acid residues of SEQ ID NO: 32.

TABLE 3
anti-CD3 scFv light chain variable domain,
heavy chain variable domain sequences,
and fulll ength sequence

| | Amino Acid | SEQ |
|---|---|---|
| Construct | Sequence | ID |
| Description | (N to C) | NO: |
| CD3: LC | QTVVTQEPSLTVSPGGTVTLTCGS | 7 |
| | STGAVTSGNYPNWVQQKPGQAPR | |
| | GLIGGTKFLAPGTPARFSGSLLG | |
| | GKAALTLSGVQPEDEAEYYCVL | |
| | WYSNRWVFGGGTKLTVL | |
| CD3: HC | EVQLVESGGGLVQPGGSLKLSC | 8 |
| | AASGFTFNKYAMNWVRQAPGKG | |
| | LEWVARIRSKYNNYATYYADSV | |
| | KDRFTISRDDSKNTAYLQMNNLK | |
| | TEDTAVYYCVRHGNFGNSYISY | |
| | WAYWGQGTLVTVSS | |
| CD3 scFv | EVQLVESGGGLVQPGGSLKLSCA | 9 |
| (SP34.185) | ASGFTFNKYAMNWVRQAPGKG | |
| | LEWVARIRSKYNNYATYYADSV | |
| | KDRFTISRDDSKNTAYLQMNNLK | |
| | TEDTAVYYCVRHGNFGNSYISY | |
| | WAYWGQGTLVTVSSGGGGSGGG | |
| | GSGGGGSQTVVTQEPSLTVSPGG | |
| | TVTLTCGSSTGAVTSGNYPNWV | |
| | QQKPGQAPRGLIGGTKFLAPGTP | |
| | ARFSGSLLGGKAALTLSGVQPED | |
| | EAEYYCVLWYSNRWVFGGGTKL | |
| | TVL | |

Antigen Binding Fragment (Fab) or Fab′ that Bind to TROP2

In some embodiments, the antigen binding fragment (Fab) or Fab′ that binds to TROP2 comprises a Fab light chain polypeptide chain and a Fab heavy chain polypeptide. In some embodiments, the Fab light chain polypeptide comprises a Fab light chain variable domain. In some embodiments, the Fab heavy chain polypeptide comprises a Fab heavy chain variable domain. In some embodiments, the Fab heavy chain variable domain comprises at least one, two, or three complementarity determining regions (CDR)s disclosed in Table 4 or a sequence substantially identical thereto (e.g., a sequence that has at least 90%, 95%, 96%, 97%, 98%, or 99% sequence identity). In some embodiments, the Fab light chain variable domain comprises at least one, two, or three complementarity determining regions (CDR)s disclosed in Table 5 or a sequence substantially identical thereto (e.g., a sequence that has at least 90%, 95%, 96%, 97%, 98%, or 99% sequence identity).

In some embodiments, the Fab heavy chain variable domain comprises at least one, two, or three complementarity determining regions (CDR)s disclosed in Table 4 or a sequence substantially identical thereto (e.g., a sequence that has at least 90%, 95%, 96%, 97%, 98%, or 99% sequence identity); and the Fab light chain variable domain comprises at least one, two, or three complementarity determining regions (CDR)s disclosed in Table 5 or a sequence substantially identical thereto (e.g., a sequence that has at least 90%, 95%, 96%, 97%, 98%, or 99% sequence identity).

In some embodiments, the Fab heavy chain variable domain comprises complementarity determining regions (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3.

In some embodiments, the Fab heavy chain variable domain comprises complementarity determining regions (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3.

In some embodiments, the Fab heavy chain variable domain comprises complementarity determining regions (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15.

In some embodiments, the Fab heavy chain variable domain comprises complementarity determining regions (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; and the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15.

TABLE 4
anti-TROP2 Fab heavy chain variable domain
complementarity determining regions (CDR)s

| | Amino Acid | SEQ |
|---|---|---|
| Construct | Sequence | ID |
| Description | (N to C) | NO: |
| TROP2: HC: CDR1 | GYTFTNYG | 10 |
| TROP2: HC: CDR2 | INTYTGEP | 11 |
| TROP2: HC: CDR3 | ARGGFGSSYWYFDV | 12 |

TABLE 5
anti-TROP2 Fab light chain variable domain
complementarity determining regions (CDR)s

| | Amino Acid | SEQ |
|---|---|---|
| Construct | Sequence | ID |
| Description | (N to C) | NO: |
| TROP2: LC: CDR1 | QDVSIA | 13 |
| TROP2: LC: CDR2 | SAS | 14 |
| TROP2: LC: CDR3 | QQHYITPLT | 15 |

In some embodiments, the Fab heavy chain polypeptide comprises an amino acid sequence that has at least 70% sequence identity to the amino acid sequence according to SEQ ID NO: 17. In some embodiments, the Fab heavy chain polypeptide comprises an amino acid sequence that has at least 80% sequence identity to the amino acid sequence according to SEQ ID NO: 17. In some embodiments, the Fab heavy chain polypeptide comprises an amino acid sequence that has at least 85% sequence identity to the amino acid sequence according to SEQ ID NO: 17. In some embodiments, the Fab heavy chain polypeptide comprises an amino acid sequence that has at least 90% sequence identity to the amino acid sequence according to SEQ ID NO: 17. In some embodiments, the Fab heavy chain polypeptide comprises an amino acid sequence that has at least 91% sequence identity to the amino acid sequence according to SEQ ID NO: 17. In some embodiments, the Fab heavy chain polypeptide comprises an amino acid sequence that has at least 92% sequence identity to the amino acid sequence according to SEQ ID NO: 17. In some embodiments, the Fab heavy chain polypeptide comprises an amino acid sequence that has at least 93% sequence identity to the amino acid sequence according to SEQ ID NO: 17. In some embodiments, the Fab heavy chain polypeptide comprises an amino acid sequence that has at least 94% sequence identity to the amino acid sequence according to SEQ ID NO: 17. In some embodiments, the Fab heavy chain polypeptide comprises an amino acid sequence that has at least 95% sequence identity to the amino acid sequence according to SEQ ID NO: 17. In some embodiments, the Fab heavy chain polypeptide comprises an amino acid sequence that has at least 96% sequence identity to the amino acid sequence according to SEQ ID NO: 17. In some embodiments, the Fab heavy chain polypeptide comprises an amino acid sequence that has at least 97% sequence identity to the amino acid sequence according to SEQ ID NO: 17. In some embodiments, the Fab heavy chain polypeptide comprises an amino acid sequence that has at least 98% sequence identity to the amino acid sequence according to SEQ ID NO: 17. In some embodiments, the Fab heavy chain polypeptide comprises an amino acid sequence that has at least 99% sequence identity to the amino acid sequence according to SEQ ID NO: 17. In some embodiments, the Fab heavy chain polypeptide comprises an amino acid sequence according to SEQ ID NO: 17.

In some embodiments, the Fab heavy chain polypeptide comprises an amino acid sequence of at least 100 consecutive amino acid residues of SEQ ID NO: 17. In some embodiments, the Fab heavy chain polypeptide comprises an amino acid sequence of at least 150 consecutive amino acid residues of SEQ ID NO: 17. In some embodiments, the Fab heavy chain polypeptide comprises an amino acid sequence of at least 175 consecutive amino acid residues of SEQ ID NO: 17. In some embodiments, the Fab heavy chain polypeptide comprises an amino acid sequence of at least 200 consecutive amino acid residues of SEQ ID NO: 17. In some embodiments, the Fab heavy chain polypeptide comprises an amino acid sequence of at least 210 consecutive amino acid residues of SEQ ID NO: 17. In some embodiments, the Fab heavy chain polypeptide comprises an amino acid sequence of at least 215 consecutive amino acid residues of SEQ ID NO: 17. In some embodiments, the Fab heavy chain polypeptide comprises an amino acid sequence of at least 220 consecutive amino acid residues of SEQ ID NO: 17.

In some embodiments, the Fab heavy chain polypeptide comprises an amino acid sequence of at least 100 consecutive amino acid residues of SEQ ID NO: 17, and has at least 80% sequence identity to the at least 100 consecutive amino acid residues of SEQ ID NO: 17. In some embodiments, the Fab heavy chain polypeptide comprises an amino acid sequence of at least 150 consecutive amino acid residues of SEQ ID NO: 17, and has at least 80% sequence identity to the at least 150 consecutive amino acid residues of SEQ ID NO: 17. In some embodiments, the Fab heavy chain polypeptide comprises an amino acid sequence of at least 175 consecutive amino acid residues of SEQ ID NO: 17, and has at least 80% sequence identity to the at least 175 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 80% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 80% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 80% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 80% sequence identity to the at least 220 consecutive amino acid residues of SEQ ID NO: 17.

In some embodiments, the Fab heavy chain polypeptide comprises an amino acid sequence of at least 100 consecutive amino acid residues of SEQ ID NO: 17, and has at least 90% sequence identity to the at least 100 consecutive amino acid residues of SEQ ID NO: 17. In some embodiments, the Fab heavy chain polypeptide comprises an amino acid sequence of at least 150 consecutive amino acid residues of SEQ ID NO: 17, and has at least 90% sequence identity to the at least 150 consecutive amino acid residues of SEQ ID NO: 17. In some embodiments, the Fab heavy chain polypeptide comprises an amino acid sequence of at least 175 consecutive amino acid residues of SEQ ID NO: 17, and has at least 90% sequence identity to the at least 175 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 90% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 90% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 90% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 90% sequence identity to the at least 220 consecutive amino acid residues of SEQ ID NO: 17.

In some embodiments, the Fab heavy chain polypeptide comprises an amino acid sequence of at least 100 consecutive amino acid residues of SEQ ID NO: 17, and has at least 95% sequence identity to the at least 100 consecutive amino acid residues of SEQ ID NO: 17. In some embodiments, the Fab heavy chain polypeptide comprises an amino acid sequence of at least 150 consecutive amino acid residues of SEQ ID NO: 17, and has at least 95% sequence identity to the at least 150 consecutive amino acid residues of SEQ ID NO: 17. In some embodiments, the Fab heavy chain polypeptide comprises an amino acid sequence of at least 175 consecutive amino acid residues of SEQ ID NO: 17, and has at least 95% sequence identity to the at least 175 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 95% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 95% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 95% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 95% sequence identity to the at least 220 consecutive amino acid residues of SEQ ID NO: 17.

In some embodiments, the Fab heavy chain polypeptide comprises an amino acid sequence of at least 100 consecutive amino acid residues of SEQ ID NO: 17, and has at least 99% sequence identity to the at least 100 consecutive amino acid residues of SEQ ID NO: 17. In some embodiments, the Fab heavy chain polypeptide comprises an amino acid sequence of at least 150 consecutive amino acid residues of SEQ ID NO: 17, and has at least 99% sequence identity to the at least 150 consecutive amino acid residues of SEQ ID NO: 17. In some embodiments, the Fab heavy chain polypeptide comprises an amino acid sequence of at least 175 consecutive amino acid residues of SEQ ID NO: 17, and has at least 99% sequence identity to the at least 175 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 99% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 99% sequence identity to the at least 210 consecutive amino acid residues of SEQ ID NO: 17.

In some embodiments, the Fab heavy chain polypeptide comprises an amino acid sequence of at least 215 consecutive amino acid residues of SEQ ID NO: 17, and has at least 99% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 99% sequence identity to the at least 220 consecutive amino acid residues of SEQ ID NO: 17.

In some embodiments, the Fab light chain polypeptide comprises an amino acid sequence that has at least 70% sequence identity to the amino acid sequence according to SEQ ID NO: 16. In some embodiments, the Fab light chain polypeptide comprises an amino acid sequence that has at least 80% sequence identity to the amino acid sequence according to SEQ ID NO: 16. In some embodiments, the Fab light chain polypeptide comprises an amino acid sequence that has at least 85% sequence identity to the amino acid sequence according to SEQ ID NO: 16. In some embodiments, the Fab light chain polypeptide comprises an amino acid sequence that has at least 90% sequence identity to the amino acid sequence according to SEQ ID NO: 16. In some embodiments, the Fab light chain polypeptide comprises an amino acid sequence that has at least 91% sequence identity to the amino acid sequence according to SEQ ID NO: 16. In some embodiments, the Fab light chain polypeptide comprises an amino acid sequence that has at least 92% sequence identity to the amino acid sequence according to SEQ ID NO: 16. In some embodiments, the Fab light chain polypeptide comprises an amino acid sequence that has at least 93% sequence identity to the amino acid sequence according to SEQ ID NO: 16. In some embodiments, the Fab light chain polypeptide comprises an amino acid sequence that has at least 94% sequence identity to the amino acid sequence according to SEQ ID NO: 16. In some embodiments, the Fab light chain polypeptide comprises an amino acid sequence that has at least 95% sequence identity to the amino acid sequence according to SEQ ID NO: 16. In some embodiments, the Fab light chain polypeptide comprises an amino acid sequence that has at least 96% sequence identity to the amino acid sequence according to SEQ ID NO: 16. In some embodiments, the Fab light chain polypeptide comprises an amino acid sequence that has at least 97% sequence identity to the amino acid sequence according to SEQ ID NO: 16. In some embodiments, the Fab light chain polypeptide comprises an amino acid sequence that has at least 98% sequence identity to the amino acid sequence according to SEQ ID NO: 16. In some embodiments, the Fab light chain polypeptide comprises an amino acid sequence that has at least 99% sequence identity to the amino acid sequence according to SEQ ID NO: 16. In some embodiments, the Fab light chain polypeptide comprises an amino acid sequence according to SEQ ID NO: 16.

In some embodiments, the Fab light chain polypeptide comprises an amino acid sequence of at least 100 consecutive amino acid residues of SEQ ID NO: 16. In some embodiments, the Fab light chain polypeptide comprises an amino acid sequence of at least 150 consecutive amino acid residues of SEQ ID NO: 16. In some embodiments, the Fab light chain polypeptide comprises an amino acid sequence of at least 175 consecutive amino acid residues of SEQ ID NO: 16. In some embodiments, the Fab light chain polypeptide comprises an amino acid sequence of at least 190 consecutive amino acid residues of SEQ ID NO: 16. In some embodiments, the Fab light chain polypeptide comprises an amino acid sequence of at least 200 consecutive amino acid residues of SEQ ID NO: 16. In some embodiments, the Fab light chain polypeptide comprises an amino acid sequence of at least 205 consecutive amino acid residues of SEQ ID NO: 16. In some embodiments, the Fab light chain polypeptide comprises an amino acid sequence of at least 210 consecutive amino acid residues of SEQ ID NO: 16.

In some embodiments, the Fab light chain polypeptide comprises an amino acid sequence of at least 100 consecutive amino acid residues of SEQ ID NO: 16, and has at least 80% sequence identity to the at least 100 consecutive amino acid residues of SEQ ID NO: 16. In some embodiments, the Fab light chain polypeptide comprises an amino acid sequence of at least 150 consecutive amino acid residues of SEQ ID NO: 16, and has at least 80% sequence identity to the at least 150 consecutive amino acid residues of SEQ ID NO: 16. In some embodiments, the Fab light chain polypeptide comprises an amino acid sequence of at least 175 consecutive amino acid residues of SEQ ID NO: 16, and has at least 80% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 80% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 80% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 80% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 80% sequence identity to the at least 210 consecutive amino acid residues of SEQ ID NO: 16.

In some embodiments, the Fab light chain polypeptide comprises an amino acid sequence of at least 100 consecutive amino acid residues of SEQ ID NO: 16, and has at least 90% sequence identity to the at least 100 consecutive amino acid residues of SEQ ID NO: 16. In some embodiments, the Fab light chain polypeptide comprises an amino acid sequence of at least 150 consecutive amino acid residues of SEQ ID NO: 16, and has at least 90% sequence identity to the at least 150 consecutive amino acid residues of SEQ ID NO: 16. In some embodiments, the Fab light chain polypeptide comprises an amino acid sequence of at least 175 consecutive amino acid residues of SEQ ID NO: 16, and has at least 90% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 90% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 90% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 90% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 90% sequence identity to the at least 210 consecutive amino acid residues of SEQ ID NO: 16.

In some embodiments, the Fab light chain polypeptide comprises an amino acid sequence of at least 100 consecutive amino acid residues of SEQ ID NO: 16, and has at least 95% sequence identity to the at least 100 consecutive amino acid residues of SEQ ID NO: 16. In some embodiments, the Fab light chain polypeptide comprises an amino acid sequence of at least 150 consecutive amino acid residues of SEQ ID NO: 16, and has at least 95% sequence identity to the at least 150 consecutive amino acid residues of SEQ ID NO: 16. In some embodiments, the Fab light chain polypeptide comprises an amino acid sequence of at least 175 consecutive amino acid residues of SEQ ID NO: 16, and has at least 95% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 95% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 95% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 95% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 95% sequence identity to the at least 210 consecutive amino acid residues of SEQ ID NO: 16.

In some embodiments, the Fab light chain polypeptide comprises an amino acid sequence of at least 100 consecutive amino acid residues of SEQ ID NO: 16, and has at least 99% sequence identity to the at least 100 consecutive amino acid residues of SEQ ID NO: 16. In some embodiments, the Fab light chain polypeptide comprises an amino acid sequence of at least 150 consecutive amino acid residues of SEQ ID NO: 16, and has at least 99% sequence identity to the at least 150 consecutive amino acid residues of SEQ ID NO: 16. In some embodiments, the Fab light chain polypeptide comprises an amino acid sequence of at least 175 consecutive amino acid residues of SEQ ID NO: 16, and has at least 99% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 99% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 99% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 99% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 99% sequence identity to the at least 210 consecutive amino acid residues of SEQ ID NO: 16.

In some embodiments, the Fab heavy chain polypeptide comprises an amino acid sequence that has at least 70% sequence identity to the amino acid sequence according to SEQ ID NO: 17; and the Fab light chain polypeptide comprises an amino acid sequence that has at least 70% sequence identity to the amino acid sequence according to SEQ ID NO: 16. In some embodiments, the Fab heavy chain polypeptide comprises an amino acid sequence that has at least 80% sequence identity to the amino acid sequence according to SEQ ID NO: 17; and the Fab light chain polypeptide comprises an amino acid sequence that has at least 80% sequence identity to the amino acid sequence according to SEQ ID NO: 16. In some embodiments, the Fab heavy chain polypeptide comprises an amino acid sequence that has at least 85% sequence identity to the amino acid sequence according to SEQ ID NO: 17; and the Fab light chain polypeptide comprises an amino acid sequence that has at least 85% sequence identity to the amino acid sequence according to SEQ ID NO: 16. In some embodiments, the Fab heavy chain polypeptide comprises an amino acid sequence that has at least 90% sequence identity to the amino acid sequence according to SEQ ID NO: 17; and the Fab light chain polypeptide comprises an amino acid sequence that has at least 90% sequence identity to the amino acid sequence according to SEQ ID NO: 16. In some embodiments, the Fab heavy chain polypeptide comprises an amino acid sequence that has at least 91% sequence identity to the amino acid sequence according to SEQ ID NO: 17; and the Fab light chain polypeptide comprises an amino acid sequence that has at least 91% sequence identity to the amino acid sequence according to SEQ ID NO: 16. In some embodiments, the Fab heavy chain polypeptide comprises an amino acid sequence that has at least 92% sequence identity to the amino acid sequence according to SEQ ID NO: 17; and the Fab light chain polypeptide comprises an amino acid sequence that has at least 92% sequence identity to the amino acid sequence according to SEQ ID NO: 16. In some embodiments, the Fab heavy chain polypeptide comprises an amino acid sequence that has at least 93% sequence identity to the amino acid sequence according to SEQ ID NO: 17; and the Fab light chain polypeptide comprises an amino acid sequence that has at least 93% sequence identity to the amino acid sequence according to SEQ ID NO: 16. In some embodiments, the Fab heavy chain polypeptide comprises an amino acid sequence that has at least 94% sequence identity to the amino acid sequence according to SEQ ID NO: 17; and the Fab light chain polypeptide comprises an amino acid sequence that has at least 94% sequence identity to the amino acid sequence according to SEQ ID NO: 16. In some embodiments, the Fab heavy chain polypeptide comprises an amino acid sequence that has at least 95% sequence identity to the amino acid sequence according to SEQ ID NO: 17; and the Fab light chain polypeptide comprises an amino acid sequence that has at least 95% sequence identity to the amino acid sequence according to SEQ ID NO: 16. In some embodiments, the Fab heavy chain polypeptide comprises an amino acid sequence that has at least 96% sequence identity to the amino acid sequence according to SEQ ID NO: 17; and the Fab light chain polypeptide comprises an amino acid sequence that has at least 96% sequence identity to the amino acid sequence according to SEQ ID NO: 16. In some embodiments, the Fab heavy chain polypeptide comprises an amino acid sequence that has at least 97% sequence identity to the amino acid sequence according to SEQ ID NO: 17; and the Fab light chain polypeptide comprises an amino acid sequence that has at least 97% sequence identity to the amino acid sequence according to SEQ ID NO: 16. In some embodiments, the Fab heavy chain polypeptide comprises an amino acid sequence that has at least 98% sequence identity to the amino acid sequence according to SEQ ID NO: 17; and the Fab light chain polypeptide comprises an amino acid sequence that has at least 98% sequence identity to the amino acid sequence according to SEQ ID NO: 16. In some embodiments, the Fab heavy chain polypeptide comprises an amino acid sequence that has at least 99% sequence identity to the amino acid sequence according to SEQ ID NO: 17; and the Fab light chain polypeptide comprises an amino acid sequence that has at least 99% sequence identity to the amino acid sequence according to SEQ ID NO: 16.

TABLE 6
anti-TROP2 Fab light chain polypeptide and
Fab heavy chain polypeptide sequences

| | Amino Acid | SEQ |
|---|---|---|
| Construct | Sequence | ID |
| Description | (N to C) | NO: |
| TROP2: LC | DIQLTQSPSSLSASVGDRVSITCKA | 16 |
| | SQDVSIAVAWYQQKPGKAPKLLI | |
| | YSASYRYTGVPDRFSGSGSGTDFT | |
| | LTISSLQPEDFAVYYCQQHYITPL | |
| | TFGAGTKVEIKRTVAAPSVFIFPPS | |
| | DEQLKSGTASVVCLLNNFYPREA | |
| | KVQWKVDNALQSGNSQESVTEQ | |
| | DSKDSTYSLSSTLTLSKADYEKHK | |
| | VYACEVTHQGLSSPVTKSFNRGE | |
| | C | |
| TROP2: HC | QVQLQQSGSELKKPGASVKVSCK | 17 |
| | ASGYTFTNYGMNWVKQAPGQG | |
| | LKWMGWINTYTGEPTYTDDFKG | |
| | RFAFSLDTSVSTAYLQISSLKADD | |
| | TAVYFCARGGFGSSYWYFDVW | |
| | GQGSLVTVSSASTKGPSVFPLAPS | |
| | SKSTSGGTAALGCLVKDYFPEPV | |
| | TVSWNSGALTSGVHTFPAVLQSS | |
| | GLYSLSSVVTVPSSSLGTQTYICN | |
| | VNHKPSNTKVDKKVEPKSC | |

### Linker

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 6 amino acids in length. In some embodiments, the linker is 7 amino acids in length. In some embodiments, the linker is 8 amino acids in length. In some embodiments, the linker is 9 amino acids in length. In some embodiments, the linker is 10 amino acids in length. In some embodiments, the linker is 11 amino acids in length. In some embodiments, the linker is 12 amino acids in length. In some embodiments, the linker is 13 amino acids in length. In some embodiments, the linker is 14 amino acids in length. In some embodiments, the linker is 15 amino acids in length. In some embodiments, the linker is 16 amino acids in length. In some embodiments, the linker is 17 amino acids in length. In some embodiments, the linker is 18 amino acids in length. In some embodiments, the linker is 19 amino acids in length. In some embodiments, the linker is 20 amino acids in length. In some embodiments, the linker is 21 amino acids in length. In some embodiments, the linker is 22 amino acids in length. In some embodiments, the linker is 23 amino acids in length. In some embodiments, the linker is 24 amino acids in length. In some embodiments, the linker is 25 amino acids in length. In some embodiments, the linker is 26 amino acids in length. In some embodiments, the linker is 27 amino acids in length. In some embodiments, the linker is 28 amino acids in length. In some embodiments, the linker is 29 amino acids in length. In some embodiments, the linker is 30 amino acids in length. In some embodiments, the linker comprises an amino acid sequence of SEQ ID NO: 19 (GGGGSGGGGSGGGGS) or SEQ ID NO: 18 (GGGGS). In some embodiments, the linker comprises an amino acid sequence of SEQ ID NO: 19 (GGGGSGGGGSGGGGS). In some embodiments, the linker comprises an amino acid sequence of SEQ ID NO: 18 (GGGGS).

In some embodiments, the linker connects the C-terminus of A to an N-terminus of B. In some embodiments, the linker connects the N-terminus of A to a C-terminus of B. In some embodiments, the linker connects the C-terminus of A to the N-terminus of the Fab heavy chain polypeptide. In some embodiments, the linker connects the N-terminus of A to the C-terminus of the Fab heavy chain polypeptide. In some embodiments, the linker connects the C-terminus of A to the N-terminus of the Fab light chain polypeptide. In some embodiments, the linker connects the N-terminus of A to the C-terminus of the Fab light chain polypeptide. In some embodiments, the linker connects the Fab light chain polypeptide to the scFv light chain variable domain. In some embodiments, the linker connects the Fab light chain polypeptide to the scFv heavy chain variable domain. In some embodiments, the linker connects the Fab heavy chain polypeptide to the scFv light chain variable domain. In some embodiments, the linker connects the Fab heavy chain polypeptide to the scFv heavy chain variable domain. In some embodiments, the linker connects the Fab light chain polypeptide to the N-terminus of the scFv light chain variable domain. In some embodiments, the linker connects the Fab light chain polypeptide to the C-terminus of the scFv light chain variable domain. In some embodiments, the linker connects the Fab light chain polypeptide to the N-terminus of the scFv heavy chain variable domain. In some embodiments, the linker connects the Fab light chain polypeptide to the C-terminus of the scFv heavy chain variable domain. In some embodiments, the linker connects the Fab heavy chain polypeptide to the N-terminus of the scFv light chain variable domain. In some embodiments, the linker connects the Fab heavy chain polypeptide to the C-terminus of the scFv light chain variable domain. In some embodiments, the linker connects the Fab heavy chain polypeptide to the N-terminus of the scFv heavy chain variable domain. In some embodiments, the linker connects the Fab heavy chain polypeptide to the C-terminus of the scFv heavy chain variable domain.

In some embodiments, the linker that connects the C-terminus of A to an N-terminus of D. In some embodiments, the linker connects the C-terminus of A to the N-terminus of the Fab heavy chain polypeptide. In some embodiments, the linker connects the C-terminus of A to the N-terminus of the Fab light chain polypeptide. In some embodiments, the linker connects the C-terminus of the scFv light chain variable domain to the N-terminus of the Fab heavy chain polypeptide. In some embodiments, the linker connects the C-terminus of scFv light chain variable domain to the N-terminus of the Fab light chain polypeptide. In some embodiments, the linker connects the C-terminus of the scFv heavy chain variable domain to the N-terminus of the Fab heavy chain polypeptide. In some embodiments, the linker connects the C-terminus of scFv heavy chain variable domain to the N-terminus of the Fab light chain polypeptide.

TABLE 7
Linker sequences

| Construct | Amino Acid Sequence | SEQ ID |
|---|---|---|
| Description | (N to C) | NO: |
| Linker | GGGGS | 18 |
| Linker | GGGGSGGGGSGGGGS | 19 |

Antibodies that Bind to TROP2 and CD3

In some embodiments, the linker connects the Fab heavy chain polypeptide to the C-terminus of the scFv light chain variable domain and the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 90% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 90% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 95% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 95% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 96% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 96% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 97% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 97% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 98% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 98% sequence identity to the amino acid sequence according to SEQ ID NO: 21. In some embodiments, the linker connects the Fab heavy chain polypeptide to the C-terminus of the scFv light chain variable domain and the Fab light chain polypeptide comprises an amino acid sequence that has at least 99% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 99% sequence identity to the amino acid sequence according to SEQ ID NO: 21.

In some embodiments, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv light chain variable domain comprises an amino acid sequence of at least 400 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 210 consecutive amino acid residues of SEQ ID NO: 20, and an amino acid sequence of the Fab heavy chain polypeptide that is connected to the C-terminus of the scFv light chain variable domain comprises an amino acid sequence of at least 470 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20 and has at least 80% sequence identity to the at least 100 consecutive amino acid residues of SEQ ID NO: 20 and an amino acid sequence of the Fab heavy chain polypeptide that is connected to the C-terminus of the scFv light chain variable domain comprises an amino acid sequence of at least 400 consecutive amino acid residues of SEQ ID NO: 21 and has at least 80% sequence identity to the at least 400 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 210 consecutive amino acid residues of SEQ ID NO: 20 and has at least 80% sequence identity to the at least 210 consecutive amino acid residues of SEQ ID NO: 20 and an amino acid sequence of the Fab heavy chain polypeptide that is connected to the C-terminus of the scFv light chain variable domain comprises an amino acid sequence of at least 470 consecutive amino acid residues of SEQ ID NO: 21 and has at least 80% sequence identity to the at least 470 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20 and has at least 90% sequence identity to the at least 100 consecutive amino acid residues of SEQ ID NO: 20 and an amino acid sequence of the Fab heavy chain polypeptide that is connected to the C-terminus of the scFv light chain variable domain comprises an amino acid sequence of at least 400 consecutive amino acid residues of SEQ ID NO: 21 and has at least 90% sequence identity to the at least 400 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 90% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 90% sequence identity to the at least 450 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 210 consecutive amino acid residues of SEQ ID NO: 20 and has at least 90% sequence identity to the at least 210 consecutive amino acid residues of SEQ ID NO: 20 and an amino acid sequence of the Fab heavy chain polypeptide that is connected to the C-terminus of the scFv light chain variable domain comprises an amino acid sequence of at least 470 consecutive amino acid residues of SEQ ID NO: 21 and has at least 90% sequence identity to the at least 470 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20 and has at least 95% sequence identity to the at least 100 consecutive amino acid residues of SEQ ID NO: 20 and an amino acid sequence of the Fab heavy chain polypeptide that is connected to the C-terminus of the scFv light chain variable domain comprises an amino acid sequence of at least 400 consecutive amino acid residues of SEQ ID NO: 21 and has at least 95% sequence identity to the at least 400 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 95% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 95% sequence identity to the at least 450 consecutive amino acid residues of SEQ ID NO: 21.

In some embodiments, the linker connects the Fab heavy chain polypeptide to the C-terminus of the scFv light chain variable domain and the Fab light chain polypeptide comprises an amino acid sequence of at least 210 consecutive amino acid residues of SEQ ID NO: 20 and has at least 95% sequence identity to the at least 210 consecutive amino acid residues of SEQ ID NO: 20 and an amino acid sequence of the Fab heavy chain polypeptide that is connected to the C-terminus of the scFv light chain variable domain comprises an amino acid sequence of at least 470 consecutive amino acid residues of SEQ ID NO: 21 and has at least 95% sequence identity to the at least 470 consecutive amino acid residues of SEQ ID NO: 21.

In some embodiments, the linker connects the Fab heavy chain polypeptide to the C-terminus of the scFv light chain variable domain and the Fab light chain polypeptide comprises an amino acid sequence of at least 100 consecutive amino acid residues of SEQ ID NO: 20 and has at least 99% sequence identity to the at least 100 consecutive amino acid residues of SEQ ID NO: 20 and an amino acid sequence of the Fab heavy chain polypeptide that is connected to the C-terminus of the scFv light chain variable domain comprises an amino acid sequence of at least 400 consecutive amino acid residues of SEQ ID NO: 21 and has at least 99% sequence identity to the at least 400 consecutive amino acid residues of SEQ ID NO: 21. In some embodiments, the linker connects the Fab heavy chain polypeptide to the C-terminus of the scFv light chain variable domain and the Fab light chain polypeptide comprises an amino acid sequence of at least 200 consecutive amino acid residues of SEQ ID NO: 20 and has at least 99% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 99% sequence identity to the at least 450 consecutive amino acid residues of SEQ ID NO: 21. In some embodiments, the linker connects the Fab heavy chain polypeptide to the C-terminus of the scFv light chain variable domain and the Fab light chain polypeptide comprises an amino acid sequence of at least 210 consecutive amino acid residues of SEQ ID NO: 20 and has at least 99% sequence identity to the at least 210 consecutive amino acid residues of SEQ ID NO: 20 and an amino acid sequence of the Fab heavy chain polypeptide that is connected to the C-terminus of the scFv light chain variable domain comprises an amino acid sequence of at least 470 consecutive amino acid residues of SEQ ID NO: 21 and has at least 99% sequence identity to the at least 470 consecutive amino acid residues of SEQ ID NO: 21.

In some embodiments, the linker connects the Fab heavy chain polypeptide to the C-terminus of the scFv light chain variable domain and the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 22.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 23, and an amino acid sequence of the Fab light chain polypeptide that is connected to the C-terminus of the scFv light chain variable domain comprises an amino acid sequence of at least 400 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 23, and an amino acid sequence of the Fab light chain polypeptide that is connected to the C-terminus of the scFv light chain variable domain comprises an amino acid sequence of at least 460 consecutive amino acid residues of SEQ ID NO: 22.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 23 and has at least 80% sequence identity to the at least 100 consecutive amino acid residues of SEQ ID NO: 23 and an amino acid sequence of the Fab light chain polypeptide that is connected to the C-terminus of the scFv light chain variable domain comprises an amino acid sequence of at least 400 consecutive amino acid residues of SEQ ID NO: 22 and has at least 80% sequence identity to the at least 400 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 23 and has at least 80% sequence identity to the at least 220 consecutive amino acid residues of SEQ ID NO: 23 and an amino acid sequence of the Fab light chain polypeptide that is connected to the C-terminus of the scFv light chain variable domain comprises an amino acid sequence of at least 460 consecutive amino acid residues of SEQ ID NO: 22 and has at least 80% sequence identity to the at least 460 consecutive amino acid residues of SEQ ID NO: 22.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 23 and has at least 90% sequence identity to the at least 100 consecutive amino acid residues of SEQ ID NO: 23 and an amino acid sequence of the Fab light chain polypeptide that is connected to the C-terminus of the scFv light chain variable domain comprises an amino acid sequence of at least 400 consecutive amino acid residues of SEQ ID NO: 22 and has at least 90% sequence identity to the at least 400 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 23 and has at least 90% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 90% sequence identity to the at least 450 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 23 and has at least 90% sequence identity to the at least 220 consecutive amino acid residues of SEQ ID NO: 23 and an amino acid sequence of the Fab light chain polypeptide that is connected to the C-terminus of the scFv light chain variable domain comprises an amino acid sequence of at least 460 consecutive amino acid residues of SEQ ID NO: 22 and has at least 90% sequence identity to the at least 460 consecutive amino acid residues of SEQ ID NO: 22.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 23 and has at least 95% sequence identity to the at least 100 consecutive amino acid residues of SEQ ID NO: 23 and an amino acid sequence of the Fab light chain polypeptide that is connected to the C-terminus of the scFv light chain variable domain comprises an amino acid sequence of at least 400 consecutive amino acid residues of SEQ ID NO: 22 and has at least 95% sequence identity to the at least 400 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 23 and has at least 95% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 95% sequence identity to the at least 450 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 23 and has at least 95% sequence identity to the at least 220 consecutive amino acid residues of SEQ ID NO: 23 and an amino acid sequence of the Fab light chain polypeptide that is connected to the C-terminus of the scFv light chain variable domain comprises an amino acid sequence of at least 460 consecutive amino acid residues of SEQ ID NO: 22 and has at least 95% sequence identity to the at least 460 consecutive amino acid residues of SEQ ID NO: 22.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 23 and has at least 99% sequence identity to the at least 100 consecutive amino acid residues of SEQ ID NO: 23 and an amino acid sequence of the Fab light chain polypeptide that is connected to the C-terminus of the scFv light chain variable domain comprises an amino acid sequence of at least 400 consecutive amino acid residues of SEQ ID NO: 22 and has at least 99% sequence identity to the at least 400 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 23 and has at least 99% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 99% sequence identity to the at least 450 consecutive amino acid residues of SEQ ID NO: 22. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 23 and has at least 99% sequence identity to the at least 220 consecutive amino acid residues of SEQ ID NO: 23 and an amino acid sequence of the Fab light chain polypeptide that is connected to the C-terminus of the scFv light chain variable domain comprises an amino acid sequence of at least 460 consecutive amino acid residues of SEQ ID NO: 22 and has at least 99% sequence identity to the at least 460 consecutive amino acid residues.

In some embodiments, the linker connects the Fab light chain polypeptide to the C-terminus of the scFv light chain variable domain and the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

In some embodiments, the linker connects the Fab heavy chain polypeptide to the C-terminus of the scFv heavy chain variable domain and the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 80% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 90% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 90% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 95% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 95% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 96% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 96% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 97% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 97% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 98% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 98% sequence identity to the amino acid sequence according to SEQ ID NO: 33. In some embodiments, the linker connects the Fab heavy chain polypeptide to the C-terminus of the scFv heavy chain variable domain and the Fab light chain polypeptide comprises an amino acid sequence that has at least 99% sequence identity to the amino acid sequence according to SEQ ID NO: 20, and an amino acid sequence of the Fab heavy chain polypeptide that is connected to the C-terminus of the scFv heavy chain variable domain comprises an amino acid sequence that has at least 99% sequence identity to the amino acid sequence according to SEQ ID NO: 33.

In some embodiments, the linker connects the Fab heavy chain polypeptide to the C-terminus of the scFv heavy chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv heavy chain variable domain comprises an amino acid sequence of at least 400 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv heavy chain variable domain comprises an amino acid sequence of at least 450 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 210 consecutive amino acid residues of SEQ ID NO: 20, and an amino acid sequence of the Fab heavy chain polypeptide that is connected to the C-terminus of the scFv heavy chain variable domain comprises an amino acid sequence of at least 470 consecutive amino acid residues of SEQ ID NO: 33.

In some embodiments, the linker connects the Fab heavy chain polypeptide to the C-terminus of the scFv heavy chain variable domain and wherein the Fab light chain polypeptide comprises an amino acid sequence of at least 100 consecutive amino acid residues of SEQ ID NO: 20 and has at least 80% sequence identity to the at least 100 consecutive amino acid residues of SEQ ID NO: 20 and an amino acid sequence of the Fab heavy chain polypeptide that is connected to the C-terminus of the scFv heavy chain variable domain comprises an amino acid sequence of at least 400 consecutive amino acid residues of SEQ ID NO: 33 and has at least 80% sequence identity to the at least 400 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv heavy chain variable domain comprises an amino acid sequence of at least 450 consecutive amino acid residues of SEQ ID NO: 33 and has at least 80% sequence identity to the at least 450 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 210 consecutive amino acid residues of SEQ ID NO: 20 and has at least 80% sequence identity to the at least 210 consecutive amino acid residues of SEQ ID NO: 20 and an amino acid sequence of the Fab heavy chain polypeptide that is connected to the C-terminus of the scFv heavy chain variable domain comprises an amino acid sequence of at least 470 consecutive amino acid residues of SEQ ID NO: 33 and has at least 80% sequence identity to the at least 470 consecutive amino acid residues of SEQ ID NO: 33.

In some embodiments, the linker connects the Fab heavy chain polypeptide to the C-terminus of the scFv heavy chain variable domain and wherein the Fab light chain polypeptide comprises an amino acid sequence of at least 100 consecutive amino acid residues of SEQ ID NO: 20 and has at least 90% sequence identity to the at least 100 consecutive amino acid residues of SEQ ID NO: 20 and an amino acid sequence of the Fab heavy chain polypeptide that is connected to the C-terminus of the scFv heavy chain variable domain comprises an amino acid sequence of at least 400 consecutive amino acid residues of SEQ ID NO: 33 and has at least 90% sequence identity to the at least 400 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 200 consecutive amino acid residues of SEQ ID NO: 20 and has at least 90% sequence identity to the at least 200 consecutive amino acid residues of SEQ ID NO: 20 and an amino acid sequence of the Fab heavy chain polypeptide that is connected to the C-terminus of the scFv heavy chain variable domain comprises an amino acid sequence of at least 450 consecutive amino acid residues of SEQ ID NO: 33 and has at least 90% sequence identity to the at least 450 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 210 consecutive amino acid residues of SEQ ID NO: 20 and has at least 90% sequence identity to the at least 210 consecutive amino acid residues of SEQ ID NO: 20 and an amino acid sequence of the Fab heavy chain polypeptide that is connected to the C-terminus of the scFv heavy chain variable domain comprises an amino acid sequence of at least 470 consecutive amino acid residues of SEQ ID NO: 33 and has at least 90% sequence identity to the at least 470 consecutive amino acid residues of SEQ ID NO: 33.

In some embodiments, the linker connects the Fab heavy chain polypeptide to the C-terminus of the scFv heavy chain variable domain and wherein the Fab light chain polypeptide comprises an amino acid sequence of at least 100 consecutive amino acid residues of SEQ ID NO: 20 and has at least 95% sequence identity to the at least 100 consecutive amino acid residues of SEQ ID NO: 20 and an amino acid sequence of the Fab heavy chain polypeptide that is connected to the C-terminus of the scFv heavy chain variable domain comprises an amino acid sequence of at least 400 consecutive amino acid residues of SEQ ID NO: 33 and has at least 95% sequence identity to the at least 400 consecutive amino acid residues of SEQ ID NO: 33. In some embodiments, the linker connects the Fab heavy chain polypeptide to the C-terminus of the scFv heavy chain variable domain and wherein the Fab light chain polypeptide comprises an amino acid sequence of at least 200 consecutive amino acid residues of SEQ ID NO: 20 and has at least 95% sequence identity to the at least 200 consecutive amino acid residues of SEQ ID NO: 20 and an amino acid sequence of the Fab heavy chain polypeptide that is connected to the C-terminus of the scFv heavy chain variable domain comprises an amino acid sequence of at least 450 consecutive amino acid residues of SEQ ID NO: 33 and has at least 95% sequence identity to the at least 450 consecutive amino acid residues of SEQ ID NO: 33.

In some embodiments, the linker connects the Fab heavy chain polypeptide to the C-terminus of the scFv heavy chain variable domain and the Fab light chain polypeptide comprises an amino acid sequence of at least 210 consecutive amino acid residues of SEQ ID NO: 20 and has at least 95% sequence identity to the at least 210 consecutive amino acid residues of SEQ ID NO: 20 and an amino acid sequence of the Fab heavy chain polypeptide that is connected to the C-terminus of the scFv heavy chain variable domain comprises an amino acid sequence of at least 470 consecutive amino acid residues of SEQ ID NO: 33 and has at least 95% sequence identity to the at least 470 consecutive amino acid residues of SEQ ID NO: 33.

In some embodiments, the linker connects the Fab heavy chain polypeptide to the C-terminus of the scFv heavy chain variable domain and the Fab light chain polypeptide comprises an amino acid sequence of at least 100 consecutive amino acid residues of SEQ ID NO: 20 and has at least 99% sequence identity to the at least 100 consecutive amino acid residues of SEQ ID NO: 20 and an amino acid sequence of the Fab heavy chain polypeptide that is connected to the C-terminus of the scFv heavy chain variable domain comprises an amino acid sequence of at least 400 consecutive amino acid residues of SEQ ID NO: 33 and has at least 99% sequence identity to the at least 400 consecutive amino acid residues of SEQ ID NO: 33. In some embodiments, the linker connects the Fab heavy chain polypeptide to the C-terminus of the scFv heavy chain variable domain and the Fab light chain polypeptide comprises an amino acid sequence of at least 200 consecutive amino acid residues of SEQ ID NO: 20 and has at least 99% sequence identity to the at least 200 consecutive amino acid residues of SEQ ID NO: 20 and an amino acid sequence of the Fab heavy chain polypeptide that is connected to the C-terminus of the scFv heavy chain variable domain comprises an amino acid sequence of at least 450 consecutive amino acid residues of SEQ ID NO: 33 and has at least 99% sequence identity to the at least 450 consecutive amino acid residues of SEQ ID NO: 33. In some embodiments, the linker connects the Fab heavy chain polypeptide to the C-terminus of the scFv heavy chain variable domain and the Fab light chain polypeptide comprises an amino acid sequence of at least 210 consecutive amino acid residues of SEQ ID NO: 20 and has at least 99% sequence identity to the at least 210 consecutive amino acid residues of SEQ ID NO: 20 and an amino acid sequence of the Fab heavy chain polypeptide that is connected to the C-terminus of the scFv heavy chain variable domain comprises an amino acid sequence of at least 470 consecutive amino acid residues of SEQ ID NO: 33 and has at least 99% sequence identity to the at least 470 consecutive amino acid residues of SEQ ID NO: 33.

In some embodiments, the linker connects the Fab heavy chain polypeptide to the C-terminus of the scFv heavy chain variable domain and the Fab light chain polypeptide comprises an amino acid sequence according to SEQ ID NO: 20, and an amino acid sequence of the Fab heavy chain polypeptide that is connected to the C-terminus of the scFv heavy chain variable domain comprises an amino acid sequence to SEQ ID NO: 33.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 34. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 34.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 35, and an amino acid sequence of the Fab light chain polypeptide that is connected to the C-terminus of the scFv light chain variable domain comprises an amino acid sequence of at least 400 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 35, and an amino acid sequence of the Fab light chain polypeptide that is connected to the C-terminus of the scFv light chain variable domain comprises an amino acid sequence of at least 450 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 35, and an amino acid sequence of the Fab light chain polypeptide that is connected to the C-terminus of the scFv light chain variable domain comprises an amino acid sequence of at least 460 consecutive amino acid residues of SEQ ID NO: 34.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 35 and has at least 80% sequence identity to the at least 100 consecutive amino acid residues of SEQ ID NO: 35 and an amino acid sequence of the Fab light chain polypeptide that is connected to the C-terminus of the scFv light chain variable domain comprises an amino acid sequence of at least 400 consecutive amino acid residues of SEQ ID NO: 34 and has at least 80% sequence identity to the at least 400 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 35 and has at least 80% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 80% sequence identity to the at least 450 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 35 and has at least 80% sequence identity to the at least 220 consecutive amino acid residues of SEQ ID NO: 35 and an amino acid sequence of the Fab light chain polypeptide that is connected to the C-terminus of the scFv light chain variable domain comprises an amino acid sequence of at least 460 consecutive amino acid residues of SEQ ID NO: 34 and has at least 80% sequence identity to the at least 460 consecutive amino acid residues of SEQ ID NO: 34.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 35 and has at least 90% sequence identity to the at least 100 consecutive amino acid residues of SEQ ID NO: 35 and an amino acid sequence of the Fab light chain polypeptide that is connected to the C-terminus of the scFv light chain variable domain comprises an amino acid sequence of at least 400 consecutive amino acid residues of SEQ ID NO: 34 and has at least 90% sequence identity to the at least 400 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 35 and has at least 90% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 90% sequence identity to the at least 450 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 35 and has at least 90% sequence identity to the at least 220 consecutive amino acid residues of SEQ ID NO: 35 and an amino acid sequence of the Fab light chain polypeptide that is connected to the C-terminus of the scFv light chain variable domain comprises an amino acid sequence of at least 460 consecutive amino acid residues of SEQ ID NO: 34 and has at least 90% sequence identity to the at least 460 consecutive amino acid residues of SEQ ID NO: 34.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 35 and has at least 95% sequence identity to the at least 100 consecutive amino acid residues of SEQ ID NO: 35 and an amino acid sequence of the Fab light chain polypeptide that is connected to the C-terminus of the scFv light chain variable domain comprises an amino acid sequence of at least 400 consecutive amino acid residues of SEQ ID NO: 34 and has at least 95% sequence identity to the at least 400 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 35 and has at least 95% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 95% sequence identity to the at least 450 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 35 and has at least 95% sequence identity to the at least 220 consecutive amino acid residues of SEQ ID NO: 35 and an amino acid sequence of the Fab light chain polypeptide that is connected to the C-terminus of the scFv light chain variable domain comprises an amino acid sequence of at least 460 consecutive amino acid residues of SEQ ID NO: 34 and has at least 95% sequence identity to the at least 460 consecutive amino acid residues of SEQ ID NO: 34.

In some embodiments, the linker connects the Fab light chain polypeptide to the C-terminus of the scFv light chain variable domain and the Fab heavy chain polypeptide comprises an amino acid sequence of at least 100 consecutive amino acid residues of SEQ ID NO: 35 and has at least 99% sequence identity to the at least 100 consecutive amino acid residues of SEQ ID NO: 35 and an amino acid sequence of the Fab light chain polypeptide that is connected to the C-terminus of the scFv light chain variable domain comprises an amino acid sequence of at least 400 consecutive amino acid residues of SEQ ID NO: 34 and has at least 99% sequence identity to the at least 400 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 200 consecutive amino acid residues of SEQ ID NO: 35 and has at least 99% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 99% sequence identity to the at least 450 consecutive amino acid residues of SEQ ID NO: 34. In some embodiments, the linker connects the Fab light chain polypeptide to the C-terminus of the scFv light chain variable domain and the Fab heavy chain polypeptide comprises an amino acid sequence of at least 220 consecutive amino acid residues of SEQ ID NO: 35 and has at least 99% sequence identity to the at least 220 consecutive amino acid residues of SEQ ID NO: 35 and an amino acid sequence of the Fab light chain polypeptide that is connected to the C-terminus of the scFv light chain variable domain comprises an amino acid sequence of at least 460 consecutive amino acid residues of SEQ ID NO: 34 and has at least 99% sequence identity to the at least 460 consecutive amino acid residues.

In some embodiments, the linker connects the Fab light chain polypeptide to the C-terminus of the scFv light chain variable domain and the Fab heavy chain polypeptide comprises an amino acid sequence according to SEQ ID NO: 35, and an amino acid sequence of the Fab light chain polypeptide that is connected to the C-terminus of the scFv light chain variable domain comprises an amino acid sequence to SEQ ID NO: 34.

TABLE 8
Antibody sequences that bind to TROP2 and CD3

| | Amino Acid Sequence | SEQ ID |
|---|---|---|
| Construct Description | (N to C) | NO: |
| Ab-1 LC | DIQLTQSPSSLSASVGDRVSITCKASQDVSIAVAWY | 20 |
| | QQKPGKAPKLLIYSASYRYTGVPDRFSGSGSGTDFT | |
| | LTISSLQPEDFAVYYCQQHYITPLTFGAGTKVEIKR | |
| | TVAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| Ab-1 HC | EVQLVESGGGLVQPGGSLKLSCAASGFTFNKYAM | 21 |
| | NWVRQAPGKGLEWVARIRSKYNNYATYYADSVK | |
| | DRFTISRDDSKNTAYLQMNNLKTEDTAVYYCVRH | |
| | GNFGNSYISYWAYWGQGTLVTVSSGGGGSGGGGS | |
| | GGGGSQTVVTQEPSLTVSPGGTVTLTCGSSTGAVT | |
| | SGNYPNWVQQKPGQAPRGLIGGTKFLAPGTPARFS | |
| | GSLLGGKAALTLSGVQPEDEAEYYCVLWYSNRW | |
| | VFGGGTKLTVLGGGGSQVQLQQSGSELKKPGASV | |
| | KVSCKASGYTFTNYGMNWVKQAPGQGLKWMGW | |
| | INTYTGEPTYTDDFKGRFAFSLDTSVSTAYLQISSL | |
| | KADDTAVYFCARGGFGSSYWYFDVWGQGSLVTV | |
| | SSASTKGPSVFPLAPSSKSTSGGTAALGCLVKDYFP | |
| | EPVTVSWNSGALTSGVHTFPAVLQSSGLYSLSSVVT | |
| | VPSSSLGTQTYICNVNHKPSNTKVDKKVEPKSC | |
| Ab-2 LC | EVQLVESGGGLVQPGGSLKLSCAASGFTFNKYAM | 22 |
| | NWVRQAPGKGLEWVARIRSKYNNYATYYADSVK | |
| | DRFTISRDDSKNTAYLQMNNLKTEDTAVYYCVRH | |
| | GNFGNSYISYWAYWGQGTLVTVSSGGGGSGGGGS | |
| | GGGGSQTVVTQEPSLTVSPGGTVTLTCGSSTGAVT | |
| | SGNYPNWVQQKPGQAPRGLIGGTKFLAPGTPARFS | |
| | GSLLGGKAALTLSGVQPEDEAEYYCVLWYSNRW | |
| | VFGGGTKLTVLGGGGSDIQLTQSPSSLSASVGDRVS | |
| | ITCKASQDVSIAVAWYQQKPGKAPKLLIYSASYRY | |
| | TGVPDRFSGSGSGTDFTLTISSLQPEDFAVYYCQQH | |
| | YITPLTFGAGTKVEIKRTVAAPSVFIFPPSDEQLKSG | |
| | TASVVCLLNNFYPREAKVQWKVDNALQSGNSQES | |
| | VTEQDSKDSTYSLSSTLTLSKADYEKHKVYACEVT | |
| | HQGLSSPVTKSFNRGEC | |
| Ab-2 HC | QVQLQQSGSELKKPGASVKVSCKASGYTFTNYGM | 23 |
| | NWVKQAPGQGLKWMGWINTYTGEPTYTDDFKGR | |
| | FAFSLDTSVSTAYLQISSLKADDTAVYFCARGGFG | |
| | SSYWYFDVWGQGSLVTVSSASTKGPSVFPLAPSSK | |
| | STSGGTAALGCLVKDYFPEPVTVSWNSGALTSGVH | |
| | TFPAVLQSSGLYSLSSVVTVPSSSLGTQTYICNVNH | |
| | KPSNTKVDKKVEPKSC | |
| Ab-4 LC | QTVVTQEPSLTVSPGGTVTLTCRSSTGAVTTSNYA | 34 |
| SP34.194 scFv (VH-linker | NWVQQKPGQAPRGLIGGTNKRAPGTPARFSGSLLG | |
| 1-VL) + Linker 2 + TROP2 | GKAALTLSGVQPEDEAEYYCALWYSNLWVFGGG | |
| Fab LC | TKLTVLGGGGSGGGGSGGGGSEVQLVESGGGLVQ | |
| | PGGSLKLSCAASGFTFNTYAMNWVRQAPGKGLE | |
| | WVARIRSKYNNYATYYADSVKDRFTISRDDSKNT | |
| | AYLQMNNLKTEDTAVYYCVRHGNFGNSYVSWFA | |
| | YWGQGTLVTVSSGGGGSDIQLTQSPSSLSASVGDR | |
| | VSITCKASQDVSIAVAWYQQKPGKAPKLLIYSASY | |
| | RYTGVPDRFSGSGSGTDFTLTISSLQPEDFAVYYCQ | |
| | QHYITPLTFGAGTKVEIKRTVAAPSVFIFPPSDEQL | |
| | KSGTASVVCLLNNFYPREAKVQWKVDNALQSGNS | |
| | QESVTEQDSKDSTYSLSSTLTLSKADYEKHKVYAC | |
| | EVTHQGLSSPVTKSFNRGEC | |
| Ab4 HC | QVQLQQSGSELKKPGASVKVSCKASGYTFTNYGM | 35 |
| TROP2 Fab HC | NWVKQAPGQGLKWMGWINTYTGEPTYTDDFKGR | |
| | FAFSLDTSVSTAYLQISSLKADDTAVYFCARGGFG | |
| | SSYWYFDVWGQGSLVTVSSASTKGPSVFPLAPSSK | |
| | STSGGTAALGCLVKDYFPEPVTVSWNSGALTSGVH | |
| | TFPAVLQSSGLYSLSSVVTVPSSSLGTQTYICNVNH | |
| | KPSNTKVDKKVEPKSC | |

### Polynucleotides Encoding Polypeptides or Polypeptide Complexes

Disclosed herein, in some embodiments, are isolated recombinant nucleic acid molecules encoding polypeptides or polypeptide complexes as disclosed herein. Described herein, in some embodiments, are isolated recombinant nucleic acid molecules encoding polypeptides comprising an antibody that selectively binds to CD3 and TROP2.

Disclosed herein, in some embodiments, are isolated recombinant nucleic acid molecules encoding polypeptides of a polypeptide complex according to the following formula:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B. Disclosed herein, in some embodiments, are isolated recombinant nucleic acid molecules encoding polypeptides of a polypeptide complex comprising the following formula:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B. Disclosed herein, in some embodiments, are isolated recombinant nucleic acid molecules encoding polypeptides of a polypeptide complex comprising the following formula:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B. Disclosed herein, in some embodiments, are isolated recombinant nucleic acid molecules encoding polypeptides of a polypeptide complex according to the following formula:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B.

Disclosed herein, in some embodiments, are isolated recombinant nucleic acid molecules encoding polypeptides of a polypeptide complex according to the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D. Disclosed herein, in some embodiments, are isolated recombinant nucleic acid molecules encoding polypeptides of a polypeptide complex comprising the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D. Disclosed herein, in some embodiments, are isolated recombinant nucleic acid molecules encoding polypeptides of a polypeptide complex comprising the following formula:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D. Disclosed herein, in some embodiments, are isolated recombinant nucleic acid molecules encoding polypeptides of a polypeptide complex according to the following formula:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D.

Disclosed herein, in some embodiments, are isolated recombinant nucleic acid molecules encoding an isolated polypeptide comprising an amino acid according to SEQ ID NO: 20.

Disclosed herein, in some embodiments, are isolated recombinant nucleic acid molecules encoding an isolated polypeptide comprising an amino acid according to SEQ ID NO: 21.

Disclosed herein, in some embodiments, are isolated recombinant nucleic acid molecules encoding an isolated polypeptide comprising an amino acid according to SEQ ID NO: 22.

Disclosed herein, in some embodiments, are isolated recombinant nucleic acid molecules encoding an isolated polypeptide comprising an amino acid according to SEQ ID NO: 23.

Disclosed herein, in some embodiments, are isolated recombinant nucleic acid molecules encoding an isolated polypeptide comprising an amino acid according to SEQ ID NO: 33.

Disclosed herein, in some embodiments, are isolated recombinant nucleic acid molecules encoding an isolated polypeptide comprising an amino acid according to SEQ ID NO: 34.

Disclosed herein, in some embodiments, are isolated recombinant nucleic acid molecules encoding an isolated polypeptide comprising an amino acid according to SEQ ID NO: 35.

### Pharmaceutical Compositions

Disclosed herein, in some embodiments, are pharmaceutical compositions comprising: (a) isolated the polypeptides or polypeptide complexes as disclosed herein; and (b) a pharmaceutically acceptable excipient.

In some embodiments, the pharmaceutical composition comprises (a) an isolated polypeptide complex according to Formula I:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B and (b) a pharmaceutically acceptable excipient. In some embodiments, the pharmaceutical composition comprises (a) an isolated polypeptide complex comprising Formula I:

A-L-B (Formula I)

wherein A comprises a single chain variable fragment (scFv) that binds to CD3; and B comprises an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B and (b) a pharmaceutically acceptable excipient. In some embodiments, the pharmaceutical composition comprises (a) an isolated polypeptide complex comprising Formula I:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B and (b) a pharmaceutically acceptable excipient. In some embodiments, the pharmaceutical composition comprises (a) an isolated polypeptide complex according to Formula I:

A-L-B (Formula I)

wherein A is a single chain variable fragment (scFv) that binds to CD3; and B is an antigen binding fragment (Fab) or Fab′ that binds to TROP2 wherein the Fab or Fab′ comprises a Fab light chain polypeptide chain comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L is a linker that connects A to B and (b) a pharmaceutically acceptable excipient.

In some embodiments, the pharmaceutical composition comprises (a) an isolated polypeptide complex according to Formula I:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D and (b) a pharmaceutically acceptable excipient. In some embodiments, the pharmaceutical composition comprises (a) an isolated polypeptide complex comprising Formula I:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D and (b) a pharmaceutically acceptable excipient. In some embodiments, the pharmaceutical composition comprises (a) an isolated polypeptide complex comprising Formula I:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D and (b) a pharmaceutically acceptable excipient. In some embodiments, the pharmaceutical composition comprises (a) an isolated polypeptide complex according to Formula I:

A-L-D (Formula II)

wherein A is a single chain variable fragment (scFv) that binds to CD3; D is an antigen binding fragment (Fab) or Fab′ that binds to TROP2; and L is a linker that connects the C-terminus of A to an N-terminus of D and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Treatment

In some embodiments, are methods of treating cancer in a subject need in need thereof comprising administering to the subject a polypeptide or polypeptide complex as described herein. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer in a subject need in need thereof comprising administering to the subject a polypeptide or polypeptide complex as described herein. In some embodiments, are methods of treating breast cancer, lung cancer, urothelial cancer, endometrial cancer, ovarian cancer, prostate cancer, pancreatic cancer, gastric cancer, colon cancer, head and neck cancer, and glioma in a subject need in need thereof comprising administering to the subject a polypeptide or polypeptide complex as described herein.

Production of Antibodies that Bind to TROP2 and CD3

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M 11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to TROP2 as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EMBODIMENTS

Embodiment 1 comprises an isolated polypeptide complex according to the following formula:

A-L-B (Formula I)

wherein A comprises a single chain variable fragment (scFv) that binds to CD3; B comprises an antigen binding fragment (Fab) or Fab′ that binds to TROP2 wherein the Fab or Fab′ comprises a Fab light chain polypeptide comprising a Fab light chain variable domain and a Fab heavy chain polypeptide comprising a Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3; and L comprises a linker that connects A to B.

Embodiment 2 comprises an isolated polypeptide complex of embodiment 1, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3.

Embodiment 3 comprises an isolated polypeptide complex of any one of embodiments 1-2, wherein the scFv comprises a scFv light chain variable domain and a scFv heavy chain variable domain.

Embodiment 4 comprises an isolated polypeptide complex of any one of embodiments 1-3, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3.

Embodiment 5 comprises an isolated polypeptide complex of any one of embodiments 1-4, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3.

Embodiment 6 comprises an isolated polypeptide complex of any one of embodiments 1-5, wherein the Fab heavy chain polypeptide comprises an amino acid sequence that has at least 80% sequence identity to the amino acid sequence according to SEQ ID NO: 17.

Embodiment 7 comprises an isolated polypeptide complex of any one of embodiments 1-6, wherein the Fab heavy chain polypeptide comprises an amino acid sequence of at least 100 consecutive amino acid residues of SEQ ID NO: 17.

Embodiment 8 comprises an isolated polypeptide complex of any one of embodiments 1-7, wherein the Fab heavy chain polypeptide comprises an amino acid sequence of at least 200 consecutive amino acid residues of SEQ ID NO: 17.

Embodiment 9 comprises an isolated polypeptide complex of any one of embodiments 1-8, wherein the Fab heavy chain polypeptide comprises an amino acid sequence of at least 200 consecutive amino acid residues of SEQ ID NO: 17 and has at least 80% sequence identity to the at least 200 consecutive amino acid residues of SEQ ID NO: 17.

Embodiment 10 comprises an isolated polypeptide complex of any one of embodiments 1-9, wherein the Fab heavy chain polypeptide comprises an amino acid sequence according to SEQ ID NO: 17.

Embodiment 11 comprises an isolated polypeptide complex of any one of embodiments 1-10, wherein the Fab light chain polypeptide comprises an amino acid sequence that has at least 80% sequence identity to the amino acid sequence according to SEQ ID NO: 16.

Embodiment 12 comprises an isolated polypeptide complex of any one of embodiments 1-11, wherein the Fab light chain polypeptide comprises an amino acid sequence of at least 100 consecutive amino acid residues of SEQ ID NO: 16

Embodiment 13 comprises an isolated polypeptide complex of any one of embodiments 1-12, wherein the Fab light chain polypeptide comprises an amino acid sequence of at least 200 consecutive amino acid residues of SEQ ID NO: 16.

Embodiment 14 comprises an isolated polypeptide complex of any one of embodiments 1-13, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16.

Embodiment 15 comprises an isolated polypeptide complex of any one of embodiments 1-14, wherein the Fab light chain polypeptide comprises an amino acid sequence according to SEQ ID NO: 16.

Embodiment 16 comprises an isolated polypeptide complex of any one of embodiments 1-15, wherein the scFv heavy chain variable domain comprises an amino acid sequence that has at least 80% sequence identity to the amino acid sequence according to SEQ ID NO: 8.

Embodiment 17 comprises an isolated polypeptide complex of any one of embodiments 1-16, wherein the scFv heavy chain variable domain comprises an amino acid sequence of at least 50 consecutive amino acid residues of SEQ ID NO: 8.

Embodiment 18 comprises an isolated polypeptide complex of any one of embodiments 1-17, wherein the scFv heavy chain variable domain comprises an amino acid sequence of at least 100 consecutive amino acid residues of SEQ ID NO: 8.

Embodiment 19 comprises an isolated polypeptide complex of any one of embodiments 1-18, wherein the scFv heavy chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 20 comprises an isolated polypeptide complex of any one of embodiments 1-19, wherein the scFv heavy chain variable domain comprises an amino acid sequence according to SEQ ID NO: 8.

Embodiment 21 comprises an isolated polypeptide complex of any one of embodiments 1-20, wherein the scFv light chain variable domain comprises an amino acid sequence that has at least 80% sequence identity to the amino acid sequence according to SEQ ID NO: 7.

Embodiment 22 comprises an isolated polypeptide complex of any one of embodiments 1-21, wherein the scFv light chain variable domain comprises an amino acid sequence of at least 50 consecutive amino acid residues of SEQ ID NO: 7.

Embodiment 23 comprises an isolated polypeptide complex of any one of embodiments 1-22, wherein the scFv light chain variable domain comprises an amino acid sequence of at least 100 consecutive amino acid residues of SEQ ID NO: 7.

Embodiment 24 comprises an isolated polypeptide complex of any one of embodiments 1-23, wherein the scFv light chain variable domain comprises an amino acid sequence of at least 100 consecutive amino acid residues of SEQ ID NO: 7 and has at least 80% sequence identity to the at least 100 consecutive amino acid residues of SEQ ID NO: 7.

Embodiment 25 comprises an isolated polypeptide complex of any one of embodiments 1-24, wherein the scFv light chain variable domain comprises an amino acid sequence according to SEQ ID NO: 7.

Embodiment 26 comprises an isolated polypeptide complex of any one of embodiments 1-25, wherein the scFv comprises an amino acid sequence that has at least 80% sequence identity to the amino acid sequence according to SEQ ID NO: 9.

Embodiment 27 comprises an isolated polypeptide complex of any one of embodiments 1-26, wherein the scFv comprises an amino acid sequence of at least 150 consecutive amino acid residues of SEQ ID NO: 9.

Embodiment 28 comprises an isolated polypeptide complex of any one of embodiments 1-27, wherein the scFv comprises an amino acid sequence of at least 225 consecutive amino acid residues of SEQ ID NO: 9.

Embodiment 29 comprises an isolated polypeptide complex of any one of embodiments 1-28, wherein the scFv comprises an amino acid sequence of at least 225 consecutive amino acid residues of SEQ ID NO: 9 and has at least 80% sequence identity to the at least 225 consecutive amino acid residues of SEQ ID NO: 9.

Embodiment 30 comprises an isolated polypeptide complex of any one of embodiments 1-29, wherein the scFv comprises an amino acid sequence according to SEQ ID NO: 9.

Embodiment 31 comprises an isolated polypeptide complex of any one of embodiments 1-30, wherein the linker connects the C-terminus of A to an N-terminus of B.

Embodiment 32 comprises an isolated polypeptide complex of any one of embodiments 1-31, wherein the linker connects the N-terminus of A to a C-terminus of B.

Embodiment 33 comprises an isolated polypeptide complex of any one of embodiments 1-32, wherein the linker connects the C-terminus of A to the N-terminus of the Fab heavy chain polypeptide.

Embodiment 34 comprises an isolated polypeptide complex of any one of embodiments 1-33, wherein the linker connects the N-terminus of A to the C-terminus of the Fab heavy chain polypeptide.

Embodiment 35 comprises an isolated polypeptide complex of any one of embodiments 1-34, wherein the linker connects the C-terminus of A to the N-terminus of the Fab light chain polypeptide.

Embodiment 36 comprises an isolated polypeptide complex of any one of embodiments 1-35, wherein the linker connects the N-terminus of A to the C-terminus of the Fab light chain polypeptide.

Embodiment 37 comprises an isolated polypeptide complex of any one of embodiments 1-36, wherein the linker connects the Fab light chain polypeptide to the scFv light chain variable domain.

Embodiment 38 comprises an isolated polypeptide complex of any one of embodiments 1-37, wherein the linker connects the Fab light chain polypeptide to the scFv heavy chain variable domain.

Embodiment 39 comprises an isolated polypeptide complex of any one of embodiments 1-38, wherein the linker connects the Fab heavy chain polypeptide to the scFv light chain variable domain.

Embodiment 40 comprises an isolated polypeptide complex of any one of embodiments 1-39, wherein the linker connects the Fab heavy chain polypeptide to the scFv heavy chain variable domain.

Embodiment 41 comprises an isolated polypeptide complex of any one of embodiments 1-40, wherein the linker connects the Fab light chain polypeptide to the N-terminus of the scFv light chain variable domain.

Embodiment 42 comprises an isolated polypeptide complex of any one of embodiments 1-41, wherein the linker connects the Fab light chain polypeptide to the C-terminus of the scFv light chain variable domain.

Embodiment 43 comprises an isolated polypeptide complex of any one of embodiments 1-42, wherein the linker connects the Fab light chain polypeptide to the N-terminus of the scFv heavy chain variable domain.

Embodiment 44 comprises an isolated polypeptide complex of any one of embodiments 1-43, wherein the linker connects the Fab light chain polypeptide to the C-terminus of the scFv heavy chain variable domain.

Embodiment 45 comprises an isolated polypeptide complex of any one of embodiments 1-44, wherein the linker connects the Fab heavy chain polypeptide to the N-terminus of the scFv light chain variable domain.

Embodiment 46 comprises an isolated polypeptide complex of any one of embodiments 1-45, wherein the linker connects the Fab heavy chain polypeptide to the C-terminus of the scFv light chain variable domain.

Embodiment 47 comprises an isolated polypeptide complex of any one of embodiments 1-46, wherein the linker connects the Fab heavy chain polypeptide to the N-terminus of the scFv heavy chain variable domain.

Embodiment 48 comprises an isolated polypeptide complex of any one of embodiments 1-47, wherein the linker connects the Fab heavy chain polypeptide to the C-terminus of the scFv heavy chain variable domain.

Embodiment 49 comprises an isolated polypeptide complex of any one of embodiments 1-48, wherein the linker is at least 5 amino acids in length.

Embodiment 50 comprises an isolated polypeptide complex of any one of embodiments 1-49, wherein the linker is no more than 30 amino acids in length.

Embodiment 51 comprises an isolated polypeptide complex of any one of embodiments 1-50, wherein the linker is at least 5 amino acids and no more than 30 amino acids in length.

Embodiment 52 comprises an isolated polypeptide complex of any one of embodiments 1-51, wherein the linker is 5 amino acids in length.

Embodiment 53 comprises an isolated polypeptide complex of any one of embodiments 1-52, wherein the linker is 15 amino acids in length.

Embodiment 54 comprises an isolated polypeptide complex of any one of embodiments 1-53, wherein the linker comprises an amino acid sequence of SEQ ID NO: 19 (GGGGSGGGGSGGGGS) or SEQ ID NO: 18 (GGGGS).

Embodiment 55 comprises an isolated polypeptide complex of any one of embodiments 1-54, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 56 comprises an isolated polypeptide complex of any one of embodiments 1-55, wherein the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv light chain variable domain comprises an amino acid sequence of at least 400 consecutive amino acid residues of SEQ ID NO: 21.

Embodiment 57 comprises an isolated polypeptide complex of any one of embodiments 1-56, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

Embodiment 58 comprises an isolated polypeptide complex of any one of embodiments 1-57, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

Embodiment 59 comprises an isolated polypeptide complex of any one of embodiments 1-58, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

Embodiment 60 comprises an isolated polypeptide complex of any one of embodiments 1-59, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 61 comprises an isolated polypeptide complex of any one of embodiments 1-60, wherein the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 23, and an amino acid sequence of the Fab light chain polypeptide that is connected to the C-terminus of the scFv light chain variable domain comprises an amino acid sequence of at least 400 consecutive amino acid residues of SEQ ID NO: 22.

Embodiment 62 comprises an isolated polypeptide complex of any one of embodiments 1-61, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

Embodiment 63 comprises an isolated polypeptide complex of any one of embodiments 1-62, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

Embodiment 64 comprises an isolated polypeptide complex of any one of embodiments 1-63, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Embodiment 65 comprises an isolated polypeptide complex of any one of embodiments 1-64, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 34.

Embodiment 66 comprises an isolated polypeptide complex of any one of embodiments 1-65, wherein the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 35, and an amino acid sequence of the Fab light chain polypeptide that is connected to the C-terminus of the scFv light chain variable domain comprises an amino acid sequence of at least 400 consecutive amino acid residues of SEQ ID NO: 34.

Embodiment 67 comprises an isolated polypeptide complex of any one of embodiments 1-66, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35, and an amino acid sequence of the Fab light chain polypeptide that is connected to the C-terminus of the scFv light chain variable domain comprises an amino acid sequence of at least 450 consecutive amino acid residues of SEQ ID NO: 34.

Embodiment 68 comprises an isolated polypeptide complex of any one of embodiments 1-67, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35 and has at least 80% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 80% sequence identity to the at least 450 consecutive amino acid residues of SEQ ID NO: 34.

Embodiment 69 comprises an isolated polypeptide complex according to the following formula:

A-L-D (Formula II)

wherein A comprises a single chain variable fragment (scFv) that binds to CD3; D comprises an antigen binding fragment (Fab) or Fab′ that binds to TROP2; and L comprises a linker that connects the C-terminus of A to an N-terminus of D.

Embodiment 70 comprises an isolated polypeptide complex of any one of embodiments 1-69, wherein the Fab or Fab′ comprises a Fab light chain polypeptide chain comprising a Fab light chain variable domain and a Fab heavy chain polypeptide comprising a Fab heavy chain variable domain

Embodiment 71 comprises an isolated polypeptide complex of any one of embodiments 1-70, wherein the scFv comprises a scFv light chain variable domain and a scFv heavy chain variable domain.

Embodiment 72 comprises an isolated polypeptide complex of any one of embodiments 1-71, wherein the linker connects the C-terminus of A to the N-terminus of the Fab heavy chain polypeptide.

Embodiment 73 comprises an isolated polypeptide complex of any one of embodiments 1-72, wherein the linker connects the C-terminus of A to the N-terminus of the Fab light chain polypeptide.

Embodiment 74 comprises an isolated polypeptide complex of any one of embodiments 1-73, wherein the linker connects the C-terminus of the scFv light chain variable domain to the N-terminus of the Fab heavy chain polypeptide.

Embodiment 75 comprises an isolated polypeptide complex of any one of embodiments 1-74, wherein the linker connects the C-terminus of scFv light chain variable domain to the N-terminus of the Fab light chain polypeptide.

Embodiment 76 comprises an isolated polypeptide complex of any one of embodiments 1-75, wherein the linker connects the C-terminus of the scFv heavy chain variable domain to the N-terminus of the Fab heavy chain polypeptide.

Embodiment 77 comprises an isolated polypeptide complex of any one of embodiments 1-76, wherein the linker connects the C-terminus of scFv heavy chain variable domain to the N-terminus of the Fab light chain polypeptide.

Embodiment 78 comprises an isolated polypeptide complex of any one of embodiments 1-77, wherein the Fab heavy chain variable domain comprising complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein the CDRs comprise from 0-2 amino acid modifications in at least one of the HC-CDR1, HC-CDR2, or HC-CDR3.

Embodiment 79 comprises an isolated polypeptide complex of any one of embodiments 1-78, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of the LC-CDR1, LC-CDR2, or LC-CDR3.

Embodiment 80 comprises an isolated polypeptide complex of any one of embodiments 1-79, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3.

Embodiment 81 comprises an isolated polypeptide complex of any one of embodiments 1-80, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3.

Embodiment 82 comprises an isolated polypeptide complex of any one of embodiments 1-81, wherein the Fab heavy chain polypeptide comprises an amino acid sequence that has at least 80% sequence identity to the amino acid sequence according to SEQ ID NO: 17.

Embodiment 83 comprises an isolated polypeptide complex of any one of embodiments 1-82, wherein the Fab heavy chain polypeptide comprises an amino acid sequence of at least 100 consecutive amino acid residues of SEQ ID NO: 17.

Embodiment 84 comprises an isolated polypeptide complex of any one of embodiments 1-83, wherein the Fab heavy chain polypeptide comprises an amino acid sequence of at least 200 consecutive amino acid residues of SEQ ID NO: 17.

Embodiment 85 comprises an isolated polypeptide complex of any one of embodiments 1-84, wherein the Fab heavy chain polypeptide comprises an amino acid sequence of at least 200 consecutive amino acid residues of SEQ ID NO: 17 and has at least 80% sequence identity to the at least 200 consecutive amino acid residues of SEQ ID NO: 17.

Embodiment 86 comprises an isolated polypeptide complex of any one of embodiments 1-85, wherein the Fab heavy chain polypeptide comprises an amino acid sequence according to SEQ ID NO: 17.

Embodiment 87 comprises an isolated polypeptide complex of any one of embodiments 1-86, wherein the Fab light chain polypeptide comprises an amino acid sequence that has at least 80% sequence identity to the amino acid sequence according to SEQ ID NO: 16.

Embodiment 88 comprises an isolated polypeptide complex of any one of embodiments 1-87, wherein the Fab light chain polypeptide comprises an amino acid sequence of at least 100 consecutive amino acid residues of SEQ ID NO: 16.

Embodiment 89 comprises an isolated polypeptide complex of any one of embodiments 1-88, wherein the Fab light chain polypeptide comprises an amino acid sequence of at least 200 consecutive amino acid residues of SEQ ID NO: 16.

Embodiment 90 comprises an isolated polypeptide complex of any one of embodiments 1-89, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16.

Embodiment 91 comprises an isolated polypeptide complex of any one of embodiments 1-90, wherein the Fab light chain polypeptide comprises an amino acid sequence according to SEQ ID NO: 16.

Embodiment 92 comprises an isolated polypeptide complex of any one of embodiments 1-91, wherein the scFv heavy chain variable domain comprises an amino acid sequence that has at least 80% sequence identity to the amino acid sequence according to SEQ ID NO: 8.

Embodiment 93 comprises an isolated polypeptide complex of any one of embodiments 1-92, wherein the scFv heavy chain variable domain comprises an amino acid sequence of at least 50 consecutive amino acid residues of SEQ ID NO: 8.

Embodiment 94 comprises an isolated polypeptide complex of any one of embodiments 1-93, wherein the scFv heavy chain variable domain comprises an amino acid sequence of at least 100 consecutive amino acid residues of SEQ ID NO: 8.

Embodiment 95 comprises an isolated polypeptide complex of any one of embodiments 1-94, wherein the scFv heavy chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 96 comprises an isolated polypeptide complex of any one of embodiments 1-95, wherein the scFv heavy chain variable domain comprises an amino acid sequence according to SEQ ID NO: 8.

Embodiment 97 comprises an isolated polypeptide complex of any one of embodiments 1-96, wherein the scFv light chain variable domain comprises an amino acid sequence that has at least 80% sequence identity to the amino acid sequence according to SEQ ID NO: 7.

Embodiment 98 comprises an isolated polypeptide complex of any one of embodiments 1-97, wherein the scFv light chain variable domain comprises an amino acid sequence of at least 50 consecutive amino acid residues of SEQ ID NO: 7.

Embodiment 99 comprises an isolated polypeptide complex of any one of embodiments 1-98, wherein the scFv light chain variable domain comprises an amino acid sequence of at least 100 consecutive amino acid residues of SEQ ID NO: 7.

Embodiment 100 comprises an isolated polypeptide complex of any one of embodiments 1-99, wherein the scFv light chain variable domain comprises an amino acid sequence of at least 100 consecutive amino acid residues of SEQ ID NO: 7 and has at least 80% sequence identity to the at least 100 consecutive amino acid residues of SEQ ID NO: 7.

Embodiment 101 comprises an isolated polypeptide complex of any one of embodiments 1-100, wherein the scFv light chain variable domain comprises an amino acid sequence according to SEQ ID NO: 7.

Embodiment 102 comprises an isolated polypeptide complex of any one of embodiments 1-101, wherein the scFv comprises an amino acid sequence that has at least 80% sequence identity to the amino acid sequence according to SEQ ID NO: 9.

Embodiment 103 comprises an isolated polypeptide complex of any one of embodiments 1-102, wherein the scFv comprises an amino acid sequence of at least 150 consecutive amino acid residues of SEQ ID NO: 9.

Embodiment 104 comprises an isolated polypeptide complex of any one of embodiments 1-103, wherein the scFv comprises an amino acid sequence of at least 225 consecutive amino acid residues of SEQ ID NO: 9.

Embodiment 105 comprises an isolated polypeptide complex of any one of embodiments 1-104, wherein the scFv comprises an amino acid sequence of at least 225 consecutive amino acid residues of SEQ ID NO: 9 and has at least 80% sequence identity to the at least 225 consecutive amino acid residues of SEQ ID NO: 9.

Embodiment 106 comprises an isolated polypeptide complex of any one of embodiments 1-105, wherein the scFv comprises an amino acid sequence according to SEQ ID NO: 9.

Embodiment 107 comprises an isolated polypeptide complex of any one of embodiments 1-106, wherein the linker is at least 5 amino acids in length.

Embodiment 108 comprises an isolated polypeptide complex of any one of embodiments 1-107, wherein the linker is no more than 30 amino acids in length.

Embodiment 109 comprises an isolated polypeptide complex of any one of embodiments 1-108, wherein the linker is at least 5 amino acids and no more than 30 amino acids in length.

Embodiment 110 comprises an isolated polypeptide complex of any one of embodiments 1-109, wherein the linker is 5 amino acids in length.

Embodiment 111 comprises an isolated polypeptide complex of any one of embodiments 1-110, wherein the linker is 15 amino acids in length.

Embodiment 112 comprises an isolated polypeptide complex of any one of embodiments 1-111, wherein the linker comprises an amino acid sequence of SEQ ID NO: 19 (GGGGSGGGGSGGGGS) or SEQ ID NO: 18 (GGGGS).

Embodiment 113 comprises an isolated polypeptide complex of any one of embodiments 1-112, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 114 comprises an isolated polypeptide complex of any one of embodiments 1-113, wherein the linker connects the Fab heavy chain polypeptide to the C-terminus of the scFv light chain variable domain and wherein the Fab light chain polypeptide comprises an amino acid sequence of at least 100 consecutive amino acid residues of SEQ ID NO: 20, and an amino acid sequence of the Fab heavy chain polypeptide that is connected to the C-terminus of the scFv light chain variable domain comprises an amino acid sequence of at least 400 consecutive amino acid residues of SEQ ID NO: 21.

Embodiment 115 comprises an isolated polypeptide complex of any one of embodiments 1-114, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

Embodiment 116 comprises an isolated polypeptide complex of any one of embodiments 1-115, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

Embodiment 117 comprises an isolated polypeptide complex of any one of embodiments 1-116, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

Embodiment 118 comprises an isolated polypeptide complex of any one of embodiments 1-117, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 119 comprises an isolated polypeptide complex of any one of embodiments 1-118, wherein the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 23, and an amino acid sequence of the Fab light chain polypeptide that is connected to the C-terminus of the scFv light chain variable domain comprises an amino acid sequence of at least 400 consecutive amino acid residues of SEQ ID NO: 22.

Embodiment 120 comprises an isolated polypeptide complex of any one of embodiments 1-119, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

Embodiment 121 comprises an isolated polypeptide complex of any one of embodiments 1-120, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

Embodiment 122 comprises an isolated polypeptide complex of any one of embodiments 1-121, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Embodiment 123 comprises an isolated polypeptide complex of any one of embodiments 1-122, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 35, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 34.

Embodiment 124 comprises an isolated polypeptide complex of any one of embodiments 1-123, wherein the linker connects the Fab light chain polypeptide to the C-terminus of the scFv light chain variable domain and wherein the Fab heavy chain polypeptide comprises an amino acid sequence of at least 100 consecutive amino acid residues of SEQ ID NO: 35, and an amino acid sequence of the Fab light chain polypeptide that is connected to the C-terminus of the scFv light chain variable domain comprises an amino acid sequence of at least 400 consecutive amino acid residues of SEQ ID NO: 34.

Embodiment 125 comprises an isolated polypeptide complex of any one of embodiments 1-124, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35, and an amino acid sequence of the Fab light chain polypeptide that is connected to the C-terminus of the scFv light chain variable domain comprises an amino acid sequence of at least 450 consecutive amino acid residues of SEQ ID NO: 34.

Embodiment 126 comprises an isolated polypeptide complex of any one of embodiments 1-125, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 35 and has at least 80% sequence identity to the at least 200 consecutive amino acid residues of SEQ ID NO: 35 and an amino acid sequence of the Fab light chain polypeptide that is connected to the C-terminus of the scFv light chain variable domain comprises an amino acid sequence of at least 450 consecutive amino acid residues of SEQ ID NO: 34 and has at least 80% sequence identity to the at least 450 consecutive amino acid residues of SEQ ID NO: 34.

Embodiment 127 comprises a pharmaceutical composition comprising: the isolated polypeptide complex of any one of embodiments 1-126; and a pharmaceutically acceptable excipient.

Embodiment 128 comprises an isolated recombinant nucleic acid molecule encoding a polypeptide of the polypeptide complex of any one of embodiments 1-127.

Embodiment 129 comprises an isolated polypeptide complex of any one of embodiments 1-126 for use in a method treating breast cancer, the method comprising administering an isolated polypeptide complex of any one of embodiments 1-126 to a subject in need thereof.

Embodiment 130 comprises an isolated polypeptide complex of any one of embodiments 1-126 for use in a method treating triple-negative breast cancer, the method comprising administering an isolated polypeptide complex of any one of embodiments 1-126 to a subject in need thereof.

Embodiment 131 comprises an isolated polypeptide complex of any one of embodiments 1-126 for use in a method treating lung cancer, the method comprising administering an isolated polypeptide complex of any one of embodiments 1-126 to a subject in need thereof.

Embodiment 132 comprises an isolated polypeptide complex of any one of embodiments 1-126 for use in a method treating non-small cell lung cancer, the method comprising administering an isolated polypeptide complex of any one of embodiments 1-126 to a subject in need thereof.

Embodiment 133 comprises an isolated polypeptide complex of any one of embodiments 1-126 for use in a method treating urothelial cancer, the method comprising administering an isolated polypeptide complex of any one of embodiments 1-126 to a subject in need thereof.

Embodiment 134 comprises an isolated polypeptide complex of any one of embodiments 1-126 for use in a method treating endometrial cancer, the method comprising administering an isolated polypeptide complex of any one of embodiments 1-126 to a subject in need thereof.

Embodiment 135 comprises an isolated polypeptide complex of any one of embodiments 1-126 for use in a method treating ovarian cancer, the method comprising administering an isolated polypeptide complex of any one of embodiments 1-126 to a subject in need thereof.

Embodiment 136 comprises an isolated polypeptide complex of any one of embodiments 1-126 for use in a method treating prostate cancer, the method comprising administering an isolated polypeptide complex of any one of embodiments 1-126 to a subject in need thereof.

Embodiment 137 comprises an isolated polypeptide complex of any one of embodiments 1-126 for use in a method treating pancreatic cancer, the method comprising administering an isolated polypeptide complex of any one of embodiments 1-126 to a subject in need thereof.

Embodiment 138 comprises an isolated polypeptide complex of any one of embodiments 1-126 for use in a method treating gastric cancer, the method comprising administering an isolated polypeptide complex of any one of embodiments 1-126 to a subject in need thereof.

Embodiment 139 comprises an isolated polypeptide complex of any one of embodiments 1-126 for use in a method treating colon cancer, the method comprising administering an isolated polypeptide complex of any one of embodiments 1-126 to a subject in need thereof.

Embodiment 140 comprises an isolated polypeptide complex of any one of embodiments 1-126 for use in a method treating head and neck cancer, the method comprising administering an isolated polypeptide complex of any one of embodiments 1-126 to a subject in need thereof.

Embodiment 141 comprises an isolated polypeptide complex of any one of embodiments 1-126 for use in a method treating glioma, the method comprising administering an isolated polypeptide complex of any one of embodiments 1-126 to a subject in need thereof.

### EXAMPLES

### Example 1: TROP2 Polypeptide Complex Binding

The TROP2 polypeptide complex Ab-3 was evaluated for TROP2 and CD3E binding.

Ab-3 comprises the sequences as listed in Table 9.

TABLE 9
Ab-3 Sequences

| | | |
|---|---|---|
| | | SEQ |
| | Amino Acid Sequence | ID |
| Construct | (N to C) | NO |

anti-TROP2 Fab light chain variable domain
complementarity determining regions (CDR)s

| | | |
|---|---|---|
| TROP2: LC: CDR1 | QDVSIA | 13 |
| TROP2: LC: CDR2 | SAS | 14 |
| TROP2: LC: CDR3 | QQHYITPLT | 15 |

anti-TROP2 Fab light chain polypeptide

| | | |
|---|---|---|
| TROP2: LC | DIQLTQSPSSLSASVGDRVSITCKASQDVSIAVA | 16 |
| | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |

anti-CD3 scFv light chain variable domain
complementarity determining regions (CDR)s

| | | |
|---|---|---|
| CD3: LC: CDR1 | TGAVTTSNY | 24 |
| CD3: LC: CDR2 | GTN | 25 |
| CD3: LC: CDR3 | ALWYSNLWV | 26 |

anti-CD3 scFv heavy chain variable domain
complementarity determining regions (CDR)s

| | | |
|---|---|---|
| CD3: HC: CDR1 | GFTFNTYA | 27 |
| CD3: HC: CDR2 | IRSKYNNYAT | 28 |
| CD3: HC: CDR3 | VRHGNFGNSYVSWFAY | 29 |

anti-CD3 scFv light chain variable domain, heavy
chain variable domain sequences, and full length
sequence

| | | |
|---|---|---|
| CD3: LC | QTVVTQEPSLTVSPGGTVTLTCRSSTGAVTTSNY | 30 |
| | ANWVQQKPGQAPRGLIGGTNKRAPGTPARFSGS | |
| | LLGGKAALTLSGVQPEDEAEYYCALWYSNLWV | |
| | FGGGTKLTVL | |
| CD3: HC | EVQLVESGGGLVQPGGSLKLSCAASGFTFNTYA | 31 |
| | MNWVRQAPGKGLEWVARIRSKYNNYATYYAD | |
| | SVKsDRFTISRDDSKNTAYLQMNNLKTEDTAVY | |
| | YCVRHGNFGNSYVSWFAYWGQGTLVTVSS | |
| CD3 scFv | QTVVTQEPSLTVSPGGTVTLTCRSSTGAVTTSNY | 32 |
| (SP34.194 scFv) | ANWVQQKPGQAPRGLIGGTNKRAPGTPARFSGS | |
| | LLGGKAALTLSGVQPEDEAEYYCALWYSNLWV | |
| | FGGGTKLTVLGGGGSGGGGSGGGGSEVQLVES | |
| | GGGLVQPGGSLKLSCAASGFTFNTYAMNWVRQ | |
| | APGKGLEWVARIRSKYNNYATYYADSVKDRFT | |
| | ISRDDSKNTAYLQMNNLKTEDTAVYYCVRHGN | |
| | FGNSYVSWFAYWGQGTLVTVSS | |

Antibody sequences that bind to TROP2 and CD3

| | | |
|---|---|---|
| Ab-3 LC | DIQLTQSPSSLSASVGDRVSITCKASQDVSIAVA | 20 |
| | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| Ab-3 HC | QTVVTQEPSLTVSPGGTVTLTCRSSTGAVTTSNY | 33 |
| | ANWVQQKPGQAPRGLIGGTNKRAPGTPARFSGS | |
| | LLGGKAALTLSGVQPEDEAEYYCALWYSNLWV | |
| | FGGGTKLTVLGGGGSGGGGSGGGGSEVQLVES | |
| | GGGLVQPGGSLKLSCAASGFTFNTYAMNWVRQ | |
| | APGKGLEWVARIRSKYNNYATYYADSVKDRFT | |
| | ISRDDSKNTAYLQMNNLKTEDTAVYYCVRHGN | |
| | FGNSYVSWFAYWGQGTLVTVSSGGGGSQVQLQ | |
| | QSGSELKKPGASVKVSCKASGYTFTNYGMNWV | |
| | KQAPGQGLKWMGWINTYTGEPTYTDDFKGRF | |
| | AFSLDTSVSTAYLQISSLKADDTAVYFCARGGF | |
| | GSSYWYFDVWGQGSLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQT | |
| | YICNVNHKPSNTKVDKKVEPKSC | |

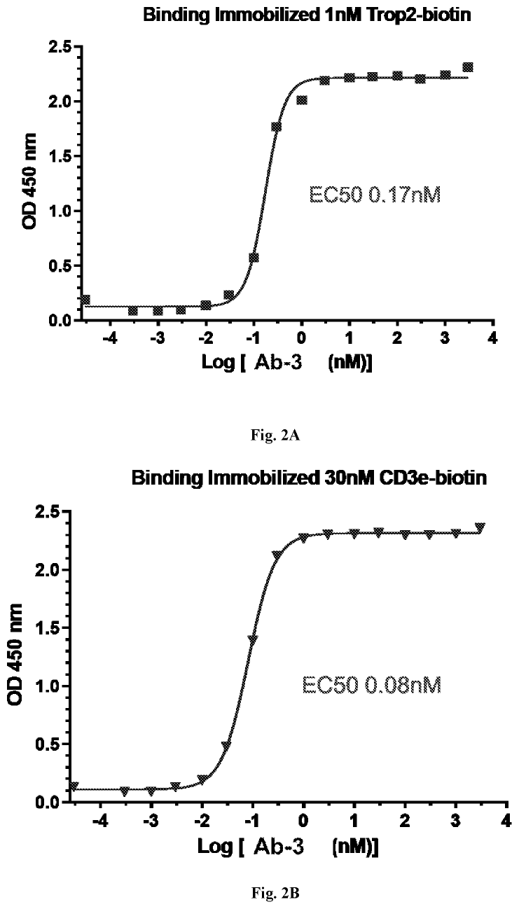

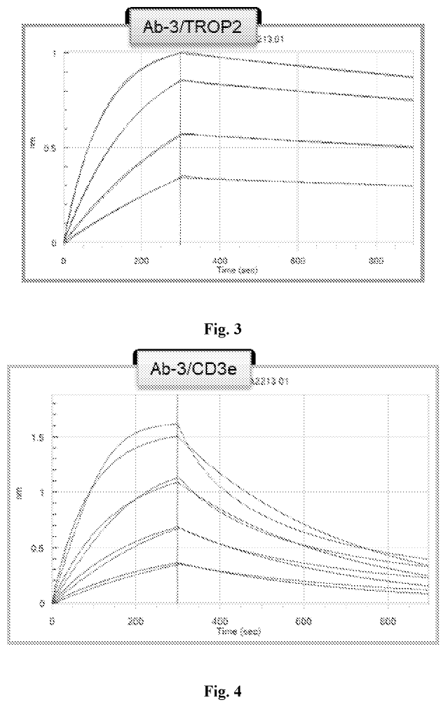

Ab-3 binding was evaluated using enzyme linked immunosorbent assays (ELISAs). Biotinylated peptides were captured on neutravidin coated plates. A secondary antibody was used to detect bound polypeptide complex. Data is seen in FIGS. 2A-2B of Ab-3 binding to TROP2 (FIG. 2A) and CD3ε (FIG. 2B). Titration data for TROP2 binding can be seen in Tables 10-11 and FIG. 3. Titration data for CD3ε can be seen in Tables 11-12 and FIG. 4.

TABLE 10

| | | |
|---|---|---|
| Step | Time | pH |

| | | | |
|---|---|---|---|
| Baseline: Octet buffer | 60 | sec | pH 7.4 |
| Load: | 300 | sec | pH 7.4 |
| 10 nM TROP2-biotin | | | |
| Biocytin quench (100 uM) | 300 | sec | pH 7.4 |
| Baseline: Octet buffer | 300 | sec | pH 7.4 |

| | | |
|---|---|---|
| Association in octet buffer | 300 sec | pH 7.4 |
| 50 nM Ab-3 | | |
| 25 nM Ab-3 | | |
| 12.5 nM Ab-3 | | |
| 6.25 nM Ab-3 | | |

| | | | |
|---|---|---|---|
| Dissociation: Octet Buffer | 900 | sec | pH 7.4 |

TABLE 11

| Sample | Loading Sample | | | | | | |
|---|---|---|---|---|---|---|---|
| ID | ID | KD (M) | KD Error | kon(1/Ms) | kdis(1/s) | Full R\hat{ }2 | Full X\hat{ }2 |
| Ab-3 | TROP2 | 1.12E−09 | 3.81E−12 | 1.99E+05 | 2.23E−04 | 0.9997 | 0.0761 |

TABLE 12

| | | |
|---|---|---|
| Step | Time | pH |

| | | | |
|---|---|---|---|
| Baseline: Octet buffer | 60 | sec | pH 7.4 |
| Load: | 300 | sec | pH 7.4 |
| 30 nM CD3e-biotin | | | |
| Biocytin quench (100 uM) | 300 | sec | pH 7.4 |
| Baseline: Octet buffer | 300 | sec | pH 7.4 |

| | | |
|---|---|---|
| Association in octet buffer | 300 sec | pH 7.4 |
| 50 nM Ab-3 | | |
| 25 nM Ab-3 | | |
| 12.5 nM Ab-3 | | |
| 6.25 nM Ab-3 | | |

| | | | |
|---|---|---|---|
| Dissociation: Octet Buffer | 600 | sec | pH 7.4 |

TABLE 13

| Sample | Loading Sample | | | | | | |
|---|---|---|---|---|---|---|---|
| ID | ID | KD (M) | KD Error | kon(1/Ms) | kdis(1/s) | Full R\hat{ }2 | Full X\hat{ }2 |
| Ab-3 | CD3e | 1.39E−08 | 2.27E−10 | 1.79E+05 | 2.50E−03 | 0.9789 | 10.2902 |

### Example 2: In Vitro Efficacy of TROP2 Polypeptide Complexes

The polypeptide complexes were next evaluated in functional in vitro tumor cell killing.

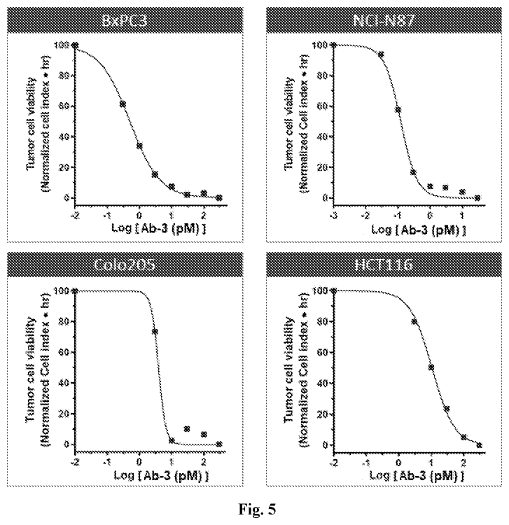

Briefly, BxPC3, NCI-N87, Colo205, and HCT116 cells were seeded onto 96 well tissue culture treated flat bottom plates and allowed to adhere overnight. The following day, culture medium and nonadherent cells were removed and replaced with fresh medium containing titrated the polypeptide complexes at concentrations indicated. T cell cytotoxicity and cell viability is seen in Table 14 and FIG. 5.

TABLE 14

| Cell-Line | TROP2 (copies/cell) | EC50 (pM) |
|---|---|---|
| BxPC3 | >250,000 | 0.50 |
| NCI-N87 | 250,000 | 0.12 |
| Colo205 | 50,000 | 4 |
| HCT116 | <10,000 | 10 |

### Example 3: TROP2 Polypeptide Complexes in Tumor Cell Killing

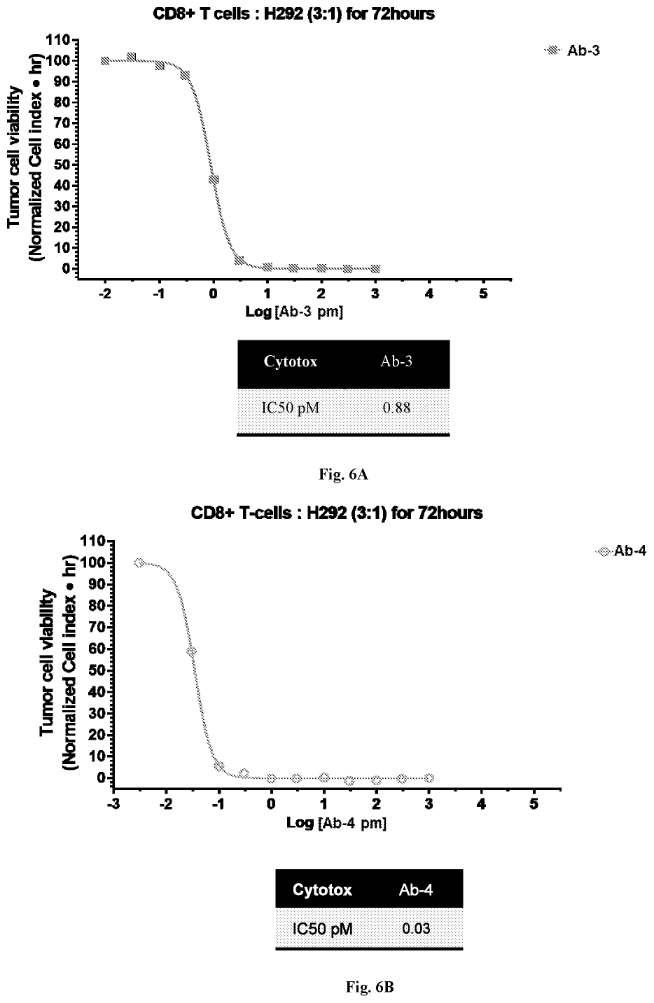

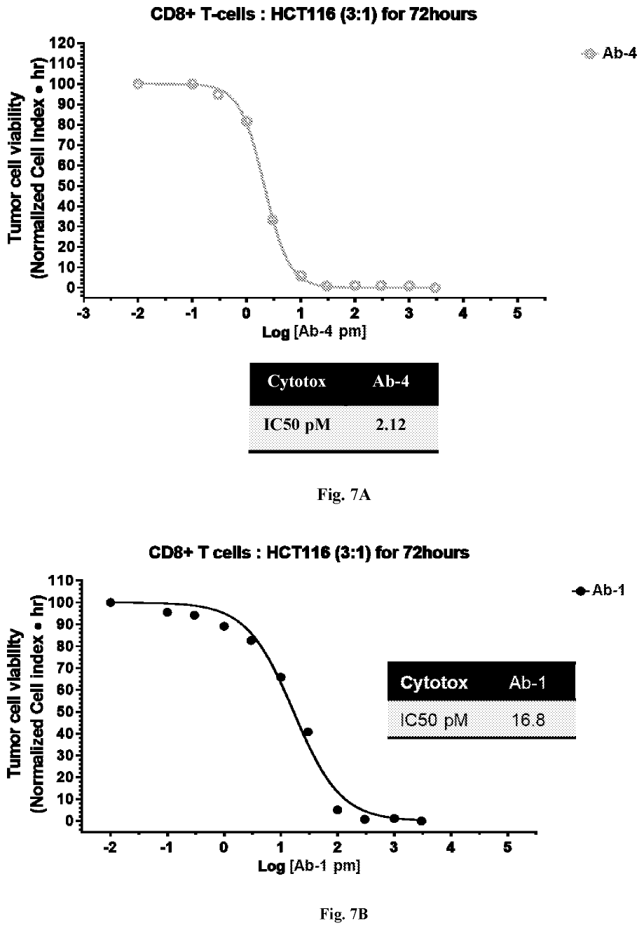

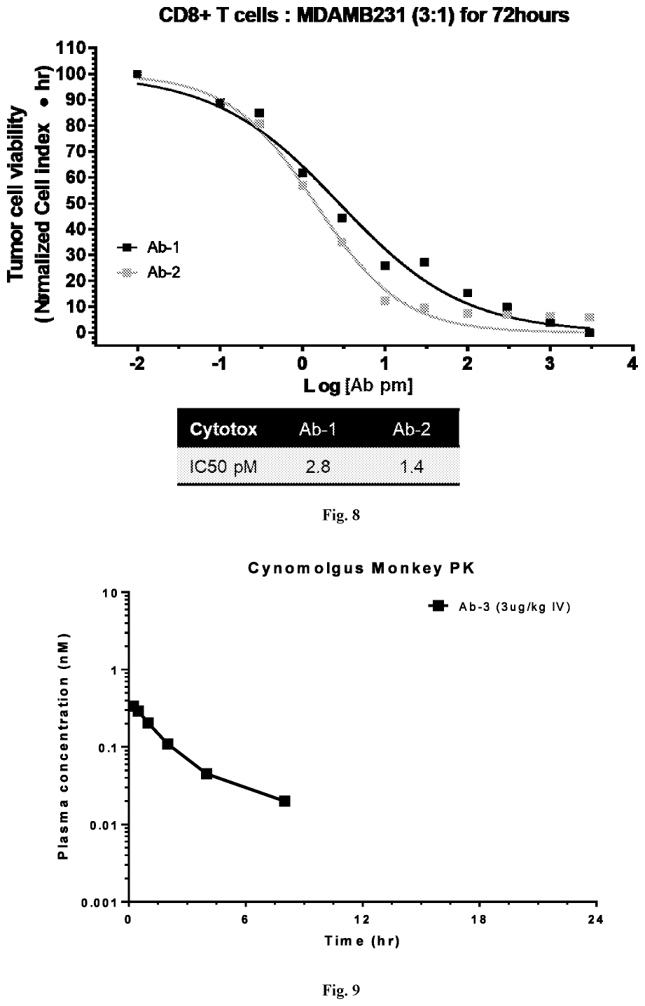

Polypeptide complexes were evaluated in a functional in vitro tumor cell killing assay using the TROP2 positive tumor cell lines HCT116, NCI-H292, and MDAMB231. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software. Data is seen for HCT116 cells (FIGS. 6A-6B), NCI-H292 cells (FIGS. 7A-7B), and MDAMB231 cells (FIG. 8).

### Example 4: Polypeptide Complex Pharmacokinetics in Cynomolgus Monkey

Pharmacokinetics and exploratory safety of polypeptide molecules were evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentration of polypeptide molecules in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7 day area under the curve (AUC) shown in Table 15. Data is also seen in FIG. 9.

TABLE 15

| | Ab-3 3 ug/kg | Units |
|---|---|---|
| C_{MAX} | 0.40 | nM |
| t_{1/2} | 1.02 | hr |
| Vd | 0.30 | L |
| VSS | 0.30 | L |
| CL | 67.31 | mL/hr/kg |
| BW | 3.00 | kg |
| 7 day AUC | 49 | nM · min |

### Example 5: Polypeptide Complexes in Cynomolgus Cytokine Release

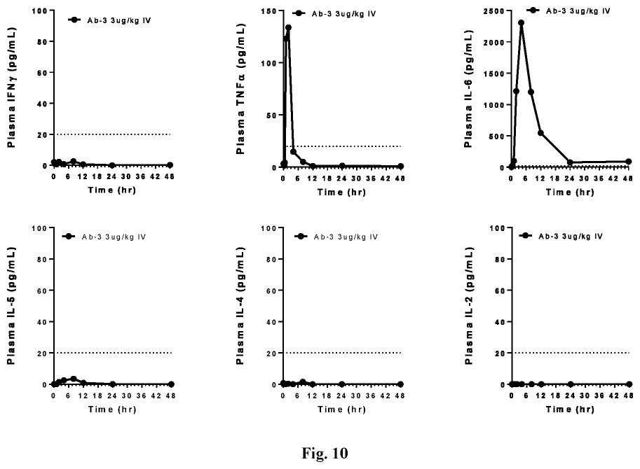

Cytokine release after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturer's instructions. Interferon gamma, tumor necrosis factor alpha, interleukin 6, interleukin 5, interleukin 4, and interleukin 2 levels in plasma were calculated relative to reference standards provided with the bead array kit. Data is seen in FIG. 10.

### Example 6: Polypeptide Complexes in Cynomolgus Toxicity

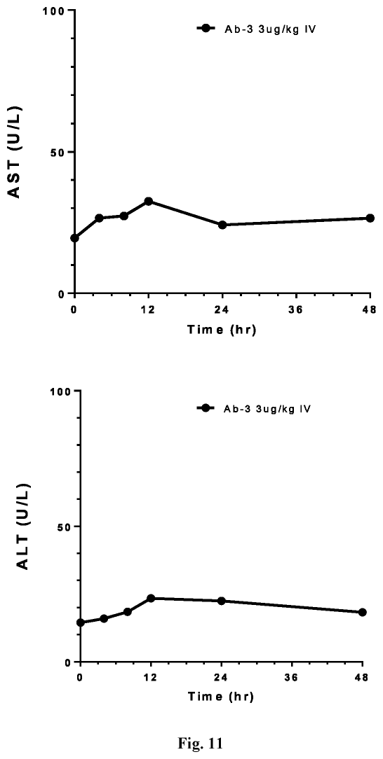

Systemic liver enzymes after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT levels were remained within the normal ranges for all timepoints tested after dosing suggesting a lack of liver toxicity. AST and ALT were quantified following the instructions provided in a commercially available kit from Millipore. AST and ALT levels were calculated according to manufacturer's instructions relative to a positive control reference standard. Data is seen in FIG. 11.

### Example 7: TROP2 Polypeptide Complex Binding for Ab-1 and Ab-2

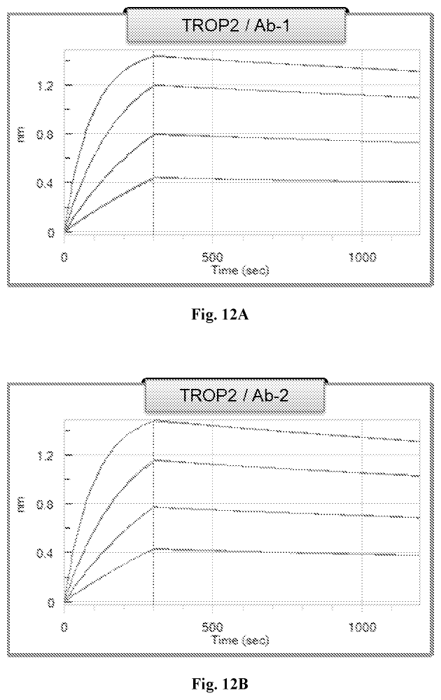

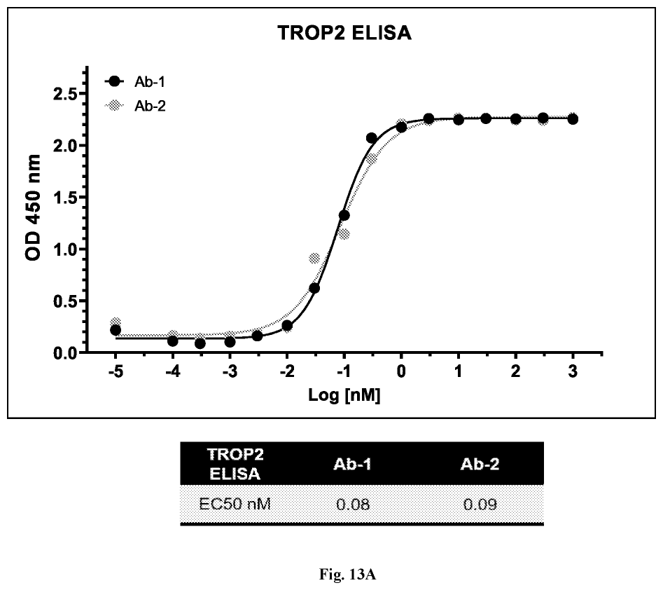

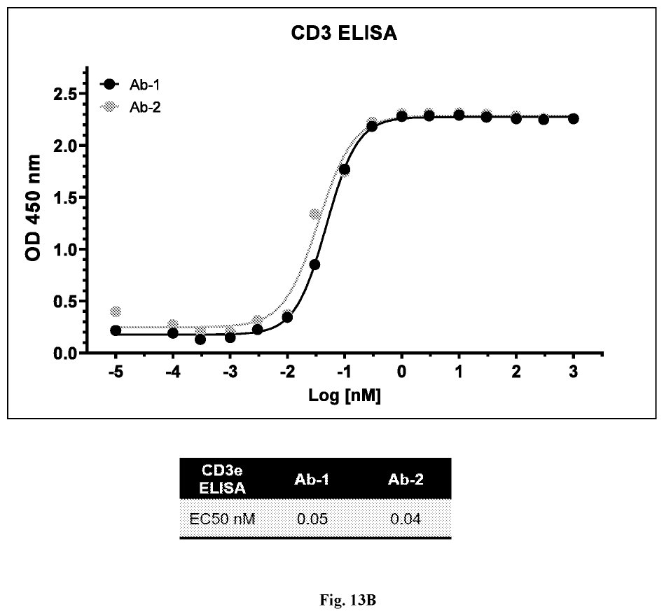

Ab-1 and Ab-2 binding were evaluated using enzyme linked immunosorbent assays (ELISAs). Biotinylated peptides were captured on neutravidin coated plates. A secondary antibody was used to detect bound polypeptide complex. A secondary antibody was used to detect bound polypeptide complex. Titration data for TROP2 binding can be seen in Tables 16-17 and FIGS. 12A-12B. Data is seen in FIGS. 13A-13B of Ab-1 and Ab-2 to TROP2-biotin (FIG. 13A) and CD3ε-biotin (FIG. 13B) measured by ELISA.

TABLE 16

| | | |
|---|---|---|
| Step | Time | pH |

| | | | |
|---|---|---|---|
| Baseline: Octet buffer | 60 | sec | pH 7.4 |
| Load: | 300 | sec | pH 7.4 |
| 10 nM Human TROP2-Biotin | | | |
| Biocytin quench (100 uM) | 300 | sec | pH 7.4 |
| Baseline: Octet buffer | 300 | sec | pH 7.4 |

| | | |
|---|---|---|
| Association in octet buffer | 300 sec | pH 7.4 |
| 50 nM Ab | | |
| 25 nM Ab | | |
| 12.5 nM Ab | | |
| 6.25 nM Ab | | |

| | | | |
|---|---|---|---|
| Dissociation: Octet buffer | 900 | sec | pH 7.4 |

TABLE 17

| Sample ID | KD (M) | kon (1/Ms) | kdis(1/s) |
|---|---|---|---|
| Ab-1 | 4.66E−10 | 2.09E+05 | 9.74E−05 |
| Ab-2 | 6.87E−10 | 1.92E+05 | 1.32E−04 |

### Example 8: Anti-Tumor Efficacy of TCE Polypeptide Complexes

TROP2 TCE polypeptide complex Ab-1 was assessed for in vivo anti-tumor efficacy in a mouse xenograft model of triple negative breast cancer (TNBC) using the MDA-MB-231 cell line. Briefly, NCG mice were subcutaneously inoculated with MDA-MB-231 tumor cells. Tumors were allowed to grow to 50 mm³ at which time mice were engrafted with human PBMCs via intravenous injection. When tumors reached 250 mm³, mice were randomized into dosing groups flowed by polypeptide, Ab-1, or vehicle intravenous administration at indicated doses every day for 10 days. Tumor volume was measured twice weekly for three weeks post dosing.

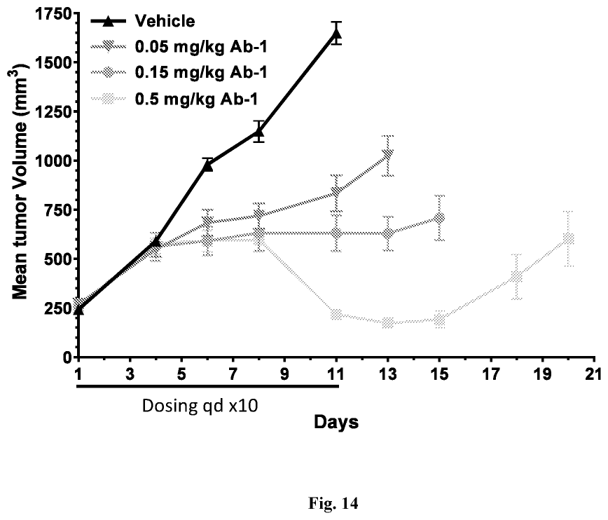

Data is seen in FIG. 14. As seen in FIG. 14, TROP2 TCE demonstrated dose dependent anti-tumor activity relative to vehicle control.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-48. (canceled)

49. An isolated polypeptide complex according to the following formula:
A-L-D (Formula II)
wherein
A comprises a single chain variable fragment (scFv) that binds to CD3;
D comprises an antigen binding fragment (Fab) or Fab′ that binds to TROP2; and
L comprises a linker that connects the C-terminus of A to an N-terminus of D and wherein the scFv comprises a scFv heavy chain variable domain and wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3 and wherein the scFv comprises a scFv light chain variable domain and the wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6.

50. The isolated polypeptide complex according to claim 49, wherein the Fab or Fab′ comprises a Fab light chain polypeptide chain comprising a Fab light chain variable domain and a Fab heavy chain polypeptide comprising a Fab heavy chain variable domain.

51. (canceled)

52. The isolated polypeptide complex according to claim 50, wherein the linker connects the C-terminus of A to the N-terminus of the Fab heavy chain polypeptide.

53. The isolated polypeptide complex according to claim 50, wherein the linker connects the C-terminus of A to the N-terminus of the Fab light chain polypeptide.

54. The isolated polypeptide complex according to claim 50, wherein the linker connects the C-terminus of the scFv light chain variable domain to the N-terminus of the Fab heavy chain polypeptide.

55. The isolated polypeptide complex according to claim 50, wherein the linker connects the C-terminus of scFv light chain variable domain to the N-terminus of the Fab light chain polypeptide.

56. The isolated polypeptide complex according to claim 5, wherein the linker connects the C-terminus of the scFv heavy chain variable domain to the N-terminus of the Fab heavy chain polypeptide.

57. The isolated polypeptide complex according to claim 50, wherein the linker connects the C-terminus of scFv heavy chain variable domain to the N-terminus of the Fab light chain polypeptide.

58. The isolated polypeptide complex according to claim 5, wherein the Fab heavy chain variable domain comprises complementarity determining regions (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12 and wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13: LC-CDR2: SEQ ID NO: 14; and LC-CDR3: SEQ ID NO: 15.

59. (canceled)

60. (canceled)

61. (canceled)

62. A pharmaceutical composition comprising:
(i) the isolated polypeptide complex of claim 49; and
(ii) a pharmaceutically acceptable excipient.

63. An isolated recombinant nucleic acid molecule encoding a polypeptide of the polypeptide complex of claim 49.

64. The isolated polypeptide complex according to claim 49, wherein the scFv heavy chain variable domain comprises an amino acid sequence that has at least 90% sequence identity to the amino acid sequence according to SEQ ID NO: 8.

65. The isolated polypeptide complex according to claim 49, wherein the scFv heavy chain variable domain comprises an amino acid sequence that has at least 95% sequence identity to the amino acid sequence according to SEQ ID NO: 8.

66. The isolated polypeptide complex according to claim 49, wherein the scFv heavy chain variable domain comprises an amino acid sequence according to SEQ ID NO: 8.

67. The isolated polypeptide complex according to claim 49, wherein the scFv light chain variable domain comprises an amino acid sequence that has at least 90% sequence identity to the amino acid sequence according to SEQ ID NO: 7.

68. The isolated polypeptide complex according to claim 49, wherein the scFv light chain variable domain comprises an amino acid sequence that has at least 95% sequence identity to the amino acid sequence according to SEQ ID NO: 7.

69. The isolated polypeptide complex according to claim 49, wherein the scFv light chain variable domain comprises an amino acid sequence according to SEQ ID NO: 7.

70. The isolated polypeptide complex according to claim 49, wherein the scFv comprises an amino acid sequence that has at least 90% sequence identity to the amino acid sequence according to SEQ ID NO: 9.

71. The isolated polypeptide complex according to claim 49, wherein the scFv comprises an amino acid sequence that has at least 95% sequence identity to the amino acid sequence according to SEQ ID NO: 9.

72. The isolated polypeptide complex according to claim 49, wherein the scFv comprises an amino acid sequence according to SEQ ID NO: 9.
